(19) United States
(12) Patent Application Publication
(10) Pub. No.: US 2005/0059018 A1
(43) Pub. Date: Mar. 17, 2005

(54) Virulence-associated nucleic acids and proteins and uses thereof

(76) Inventors: Laurence Rahme, Brookline, MA (US); Jianxin He, Malden, MA (US); Regina Baldini, Sao Paulo (BR); Jens Solsbacher, Liederbach (DE); Peter Wagner, Idstein (DE)

Correspondence Address: CLARK & ELBING LLP, 101 FEDERAL STREET, BOSTON, MA, 02110, US

(21) Appl. No.: 10/660,811

(22) Filed: Sep. 12, 2003

Related U.S. Application Data
(60) Provisional application No. 60/410,376, filed on Sep. 12, 2002.
(60) Provisional application No. 60/410,817, filed on Sep. 13, 2002.

(51) Int. Cl. C12Q 1/68; C07H 21/04; C07K 14/785

(52) U.S. Cl. 435/6; 435/69.1; 435/320.1; 435/325; 530/350; 536/23.5

(57) ABSTRACT

The present invention features methods and compositions relating to bacterial virulence polypeptides and nucleic acid sequences (e.g., DNA) encoding such polypeptides. Based on the present invention, a pathogenic infection may be treated, prevented, or reduced by administering to an infected mammal or plant a therapeutically effective amount of a composition that inhibits the expression or activity of a polypeptide encoded by the virulence factors. Also disclosed are methods for producing such polypeptides by recombinant techniques as well as methods for utilizing such polypeptides to screen for antibacterial or bacteriostatic compounds.

### CROSS-REFERENCE TO RELATED APPLICATIONS

This application claims the benefit of U.S. Provisional Application Nos. 60/410,376 (filed Sep. 12, 2002) and 60/410,817 (filed Sep. 13, 2002), each of which is hereby incorporated by reference.

### BACKGROUND OF THE INVENTION

In general, the present invention relates to nucleic acid molecules, genes, and polypeptides that are related to microbial pathogenicity.

Pathogens employ a number of genetic strategies to cause infection and, occasionally, disease in their hosts. The expression of microbial pathogenicity is dependent upon complex genetic regulatory circuits. Knowledge of the themes in microbial pathogenicity is necessary for understanding pathogen virulence mechanisms and for the development of new “anti-virulence” or “anti-pathogenic” agents, which are needed to combat infection and disease.

The mechanism of pathogenesis and the host defense is a field of intense investigation. Antibiotics have been an effective tool to treat unwanted bacterial infections. However, due to the increasing incidence of resistance to current antibiotics, new antibiotics are needed. Antibiotics that target non-essential genes are desirable because there is limited, if any, selection pressure on these genes since they are not required for the survival of the bacteria. Thus, bacteria are less likely to develop resistance to antibiotics that target these genes.

In one particular example, the opportunistic human pathogen, Pseudomonas aeruginosa, is a ubiquitous gram-negative bacterium isolated from soil, water, and plants (Palleroni, J. N. In: Bergey's Manual of Systematic Bacteriology, ed., J. G. Holt, Williams & Wilkins, Baltimore, Md., pp. 141-172, 1984). A variety of P. aeruginosa virulence factors have been described and the majority of these, such as exotoxin A, elastase, and phospholipase C, were first detected biochemically on the basis of their cytotoxic activity (Fink, R. B., Pseudomonas aeruginosa the Opportunist: Pathogenesis and Disease, Boca Raton, CRC Press Inc., 1993). Subsequently, genes corresponding to these factors or genes that regulate the expression of these factors were identified. In general, most pathogenicity-related genes in mammalian bacterial pathogens were first detected using a bio-assay. In contrast to mammalian pathogens, simple systematic genetic strategies have been routinely employed to identify pathogenicity-related genes in plant pathogens. Following random transposon-mediated mutagenesis, thousands of mutant clones of the phytopathogen are inoculated separately into individual plants to determine if they contain a mutation that affects the pathogenic interaction with the host (Boucher et al., J. Bacteriol (1987) 168:5626-5623; Comai and Kosuge, J. Bacteriol. (1982) 149:40-46; Lindgren et al., J. Bacteriol. (1986) 168:512-522; Rahme et al., J. Bacteriol. (1991) 173:575-586; Willis et al., Mol. Plant-Microbe Interact. (1990) 3:149-156). Comparable experiments using whole-animal mammalian pathogenicity models are not feasible because of the vast numbers of animals that must be subjected to pathogenic attack.

Improved methods are needed for treating, stabilizing, or preventing pathogenic infections such as bacterial and fungal infections. In particular, improved methods are needed to treat infections by opportunistic pathogens such as Pseudomonas.

### SUMMARY OF THE INVENTION

In general, this invention relates to the identification and characterization of novel virulence factors. These virulence factors can be used in a variety of applications such as (i) the generation of antibodies for diagnostic and therapeutic applications, (ii) the generation of pharmaceutical compositions, (iii) the production of diagnostic compositions for detecting pathogenic infections, (iv) the identification of compounds useful for the treatment, stabilization, or prevention of pathogenic infections in mammals (e.g. humans), (v) the treatment or prevention of pathogenic infections, (vi) the diagnosis of pathogenic infections, (vii) the identification of additional virulence factors, (viii) the identification of novel mammalian nucleic acids (e.g., human lung genes), and (ix) plant disease control.

Here, we have identified and characterized a number of nucleic acid molecules and polypeptides that are involved in conferring pathogenicity and virulence to a pathogen. Our discovery therefore provides a basis for drug-screening assays aimed at evaluating and identifying “anti-virulence” agents that are capable of blocking pathogenicity and virulence of a pathogen (e.g., by selectively switching pathogen gene expression on or off) or that inactivate or inhibit the activity of a polypeptide involved in the pathogenicity of a microbe. In turn, drugs that target these molecules are useful as anti-virulence agents.

In the first aspect, the invention features an isolated nucleic acid molecule encoding a pathogenic virulence factor protein having an amino acid sequence substantially identical (e.g., at least 25%, 50%, 80%, 90%, 95%, 99%, or even 100% identical) to any one of the amino acid sequences of any one of the ORFs described herein (for example, any one of SEQ ID NOs: 127-229 and SEQ ID NOs: 278-280). Optionally, the protein encoded by the nucleic acid binds a human protein such as a lung protein or has an Arg-Gly-Asp motif. The invention also features a nucleic acid molecule having a sequence substantially identical to any one of the polynucleotide sequence of SEQ ID NOs: 1-108, SEQ ID NOs: 119-120, and SEQ ID NOs: 281-282. Accordingly, this sequence is at least 25%, 30%, 40%, 50%, 60%, 65%, 70%, 80%, 90%, 95%, 99%, or even 100% identical to the nucleotide sequence of any of the ORFs of the invention or the complement thereof. Optionally, the nucleic acid hybridizes at high stringency to a region of any one of the polynucleotide sequences of SEQ ID NOs: 1-108, SEQ ID NOs: 119-120, and SEQ ID NOs: 281-282 or the complement thereof. For example, the nucleic acid may have a sequence complimentary to at least 50% of at least 60 nucleotides of any one of the polynucleotide sequences of the invention. Optionally, the nucleic acid of the invention contains at least 100 contiguous nucleotides (or 200, 300, 400, 500, 600, 700, 800, 900 or more contiguous nucleotides) of any one of the polynucleotide sequences of SEQ ID NOs: 1-108, SEQ ID NOs: 119-120, and SEQ ID NOs: 281-282. Preferably, the isolated nucleic acid molecule includes any of the above-described sequences or a fragment thereof and is derived from a pathogen (e.g., from a bacterial pathogen such as Pseudomonas aeruginosa, e.g., PA14).

The invention further provides an isolated nucleic acid substantially identical (e.g., at least 25%, 50%, 80%, 90%, 95%, 99%, or even 100% identical) to the nucleotide sequence of SEQ ID NOs: 109-118. Alternatively, the nucleic acid may encode a protein having an amino acid sequence at least 25% identical to any one of the amino acid sequences of SEQ ID NOs: 269-277. According to this invention, the protein encoded by this nucleic acid is expressed in the lungs of a mammal where it binds the polypeptide (SEQ ID NO: 278 or SEQ ID NO: 280) encoded by the ORF7 nucleic acid (SEQ ID NO: 119 or SEQ ID NO: 281).

The invention further features a probe, which hybridizes under hybridizing conditions to any of the nucleic acid molecules of the invention. Such a probe may be any fragment of SEQ ID NO: 1 or 2. The probe of the invention may also include at least one modified linkage (e.g., a phosphorothioate, a methylphosphonate, a phosphotriester, a phosphorodithioate, or a phosphoselenate linkage), modified nucleobase (e.g., a 5-methyl cytosine), and/or a modified sugar moiety (e.g., a 2′-O-methoxyethyl group or a 2′-O-methyl group). In one embodiment, the probe is a chimeric polynucleotide (e.g., an oligonucleotide that includes DNA residues linked together by phosphorothioate or phosphodiester linkages, flanked on each side by at least one, two, three, or four 2′-O-methyl RNA residue linked together by a phosphorothioate linkage). Thus, a probe according to this invention includes natural and non-natural oligonucleotides, both modified and unmodified, as well as oligonucleotide mimetics such as Protein Nucleic Acids, locked nucleic acids, and arabinonucleic acids. Numerous nucleobases and linkage groups may be employed in the nucleobase oligomers of the invention.

In addition, the invention includes a vector and a cell, each of which includes at least one of the isolated nucleic acid molecules of the invention. The invention further provides a method of producing a recombinant polypeptide by providing a cell transformed with a nucleic acid molecule of the invention, which is positioned for expression in the cell, culturing the transformed cell under conditions for expressing the nucleic acid molecule, and isolating a recombinant polypeptide. The invention also features recombinant polypeptides produced by the expression of an isolated nucleic acid molecule of the invention and substantially pure antibodies that specifically recognize and bind such recombinant polypeptides.

In another aspect, the invention features a substantially pure polypeptide having an amino acid sequence that is substantially identical to any one of the amino acid sequences of SEQ ID NOs: 127-229 and SEQ ID NOs: 278-280. Thus, the protein may have an amino acid sequence which is at least 25%, 30%, 40%, 50%, 60%, 65%, 70%, 80%, 90%, 95%, 99%, or even 100% identical to the sequence of any of the polypeptides of the invention. Desirably, the substantially pure polypeptide includes any of the above-described sequences or a fragment thereof and is derived from a pathogen (e.g., from a bacterial pathogen such as Pseudomonas aeruginosa, e.g., PA14). Preferably, the protein is a pathogenic virulence factor. The protein may bind a human protein such as a lung protein and optionally, the protein has an Arg-Gly-Asp motif. The protein may further be immunogenic. Desirably, the protein contains at least 100 contiguous amino acids of any of the amino acid sequences of the invention.

The present invention further features a fusion protein containing the protein of the invention and a protein of interest. Also featured is a purified antibody that specifically binds the protein of the invention.

The invention also features a substantially pure protein having an amino acid sequence at least 25% identical to any one of the amino acid sequences of SEQ ID NOs: 269-277, such that the protein binds the polypeptide (SEQ ID NO: 278 or SEQ ID NO: 280) encoded by the ORF7 nucleic acid (SEQ ID NO: 119 or SEQ ID NO: 281); although binding may occur anywhere in the mammal (e.g., any organ in the mammal), optionally, such binding occurs in the lungs.

In a related aspect, the invention features a pharmaceutical composition containing a pharmaceutically acceptable carrier in addition to a nucleic acid, a protein, a probe, or an antibody of the invention in an amount sufficient to treat, stabilize, or prevent a pathogenic infection in a mammal. For example, the pathogen may be Pseudomonas aeruginosa.

In yet another related aspect, the invention features a diagnostic composition for the detection of pathogenic bacteria (e.g., Pseudomonas aeruginosa) that contains a nucleic acid or an antibody of the invention.

In another aspect, the invention features a method for identifying a compound that decreases the expression of a virulence factor. This method involves the steps of: (a) contacting a pathogenic cell that expresses a virulence factor gene (including any one of the nucleic acid molecules of the invention) with a candidate compound; and (b) measuring the expression of this nucleic acid, such that a decrease in the expression of the virulence factor following contact with the candidate compound identifies the compound as having the ability to decrease the expression of a pathogenic virulence factor. In preferred embodiments, the pathogenic cell (e.g., Pseudomonas aeruginosa) infects a mammal (e.g., a human) or a plant.

In yet another related aspect, the invention features a method for identifying a compound which is capable of decreasing the expression of a pathogenic virulence factor (e.g., at the transcriptional or post-transcriptional levels), involving (a) providing a pathogenic cell expressing any one of the isolated nucleic acid molecules of the invention; and (b) contacting the pathogenic cell with a candidate compound, such that a decrease in the expression of the nucleic acid molecule following contact with the candidate compound identifies the compound as having the ability to decrease the expression of a pathogenic virulence factor. In preferred embodiments, the pathogenic cell (e.g., Pseudomonas aeruginosa) infects a mammal (e.g., a human) or a plant.

In yet another related aspect, the invention features a method for identifying a compound which binds a virulence factor involving (a) contacting a candidate compound with a substantially pure polypeptide including any one of the amino acid sequences of the invention under conditions that allow binding; and (b) detecting binding of the candidate compound to the polypeptide.

In yet another related aspect, the invention features a method for identifying a compound which binds a virulence factor, involving (a) contacting a candidate compound with a first protein which is a substantially pure polypeptide (including any one of the amino acid sequences of the invention) and a second protein capable of binding the polypeptide of the invention under conditions that allow binding; and (b) measuring the binding of the first protein to the second protein, such that a decrease in binding effected by the candidate compound indicates that this compound binds to the first protein or the protein of the invention. Desirably, the candidate compound inhibits virulence of a pathogen. The second protein may be an antibody or antibody fragment. Optionally, the second protein may be a human lung protein. The candidate compound may be a mammalian or plant protein. If desired, the protein of the invention or the candidate compound may be immobilized on a support or may have a detectable group. Alternatively, the candidate compound may be expressed on the surface of a phage or maybe expressed using RNA display. According to this invention, contacting of the candidate compounds with the two proteins may occur in a cell-free system or in a cell and binding of the candidate compound to the first protein may be detected using a yeast two-hybrid system.

In addition, the invention features a method of treating a pathogenic infection in a mammal involving (a) identifying a mammal having a pathogenic infection; and (b) administering to the mammal a therapeutically effective amount of a composition which inhibits the expression or activity of a polypeptide encoded by any one of the nucleic acid molecules of the invention. In preferred embodiments, the pathogen is Pseudomonas aeruginosa, such as PA14. In this regard, the composition may inhibit binding of the pathogen to a cell or cell-surface protein in the mammal. For example, the composition may contain an antibody that specifically binds the protein of the invention or a fragment thereof.

In yet another aspect, the invention features a method of treating a pathogenic infection in a mammal, involving (a) identifying a mammal having a pathogenic infection; and (b) administering to the mammal a therapeutically effective amount of a composition which binds and inhibits a polypeptide encoded by any one of the amino acid sequences of the invention. In preferred embodiments, the pathogenic infection is caused by Pseudomonas aeruginosa, e.g., PA14.

The invention further provides a method of treating a pathogenic infection in a mammal, involving (a) identifying a mammal having a pathogenic infection; and (b) administering to the mammal a therapeutically effective amount of a composition which inhibits the expression an mRNA molecule transcribed from any one of the nucleic acid molecules of the invention. In preferred embodiments, the pathogen is Pseudomonas aeruginosa, such as PA14.

In another aspect, the invention provides a method for preventing, stabilizing, or treating a pathogenic infection in a mammal by introducing into the mammal a nucleic acid of the invention or a complement thereof in an amount sufficient to specifically attenuate expression of a target nucleic acid (e.g., an mRNA) of a pathogen. The introduced nucleic acid has a nucleotide sequence that is essentially complementary (e.g., at least 20, 30, 40, 50, 60, 70, 80, 90, 95, 98, or 100% complementary) to a region of desirably at least 20 nucleotides of the target nucleic acid. According to this invention, the nucleic acid that is introduced in the mammal or the protein encoded by this nucleic acid may induce an immune response against the pathogen. Alternatively, the protein encoded by such a nucleic acid molecule may inhibit binding of the pathogen to a cell or a cell-surface protein in the mammal. Optionally, the mammal being treated may be directly administered with a therapeutically effective amount of a protein of the invention or a fragment thereof.

In desirable embodiments of the therapeutic methods of the above aspects, the mammal is a human. Other exemplary mammals include primates such as monkeys, animals of veterinary interest (e.g., cows, sheep, goats, buffalos, and horses), and domestic pets (e.g., dogs and cats). In some embodiments, the introduced nucleic acid is single stranded or double stranded (e.g., double stranded RNA).

In all foregoing aspects of the invention, the nucleic acid or polypeptide is involved in biofilm formation, surface adhesion, host invasion, toxin production, pili assembly, and/or fimbrial biogenesis. In some embodiments, a compound identified in a screening assay of the invention or administered to a mammal in a therapeutic method of the invention inhibits biofilm formation, surface adhesion, host invasion, toxin production, pili assembly, and/or fimbrial biogenesis, preferably by at least 10, 20, 30, 40, 50, 60, 70, 80, 90, 95, or 100% compared to a buffer control.

With respect to the therapeutic methods of the invention, it is not intended that the administration of compounds to a mammal be limited to a particular mode of administration, dosage, or frequency of dosing; the present invention contemplates all modes of administration, including oral, intraperitoneal, intramuscular, intravenous, intraarticular, intralesional, subcutaneous, or any other route sufficient to provide a dose adequate to prevent or treat an infection. One or more compounds may be administered to the mammal in a single dose or multiple doses. When multiple doses are administered, the doses may be separated from one another by, for example, one week, one month, one year, or ten years. It is to be understood that, for any particular subject, specific dosage regimes should be adjusted over time according to the individual need and the professional judgment of the person administering or supervising the administration of the compositions. If desired, conventional treatments can be used in combination with the compounds of the present invention.

Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid. In some embodiments, the pharmaceutical composition contains one or more pharmaceutically acceptable additives suitable for the selected route and mode of administration. These compositions may be administered by, without limitation, any parenteral route including intravenous, intra-arterial, intramuscular, subcutaneous, intradermal, intraperitoneal, intrathecal, as well as topically, orally, and by mucosal routes of delivery such as intranasal, inhalation, rectal, vaginal, buccal, and sublingual. In some embodiments, the pharmaceutical compositions of the invention are prepared for administration to vertebrate (e.g., mammalian) subjects in the form of liquids, including sterile, non-pyrogenic liquids for injection, emulsions, powders, aerosols, tablets, capsules, enteric-coated tablets, or suppositories.

Proteins substantially identical to any of the proteins of the invention (e.g., proteins having an amino acid sequence of SEQ ID NOs: 127-229 and SEQ ID NOs: 278-280) and nucleic acids substantially identical to any of the nucleic acids of the invention (e.g., nucleic acids having an nucleotide sequence of SEQ ID NOs: 1-108, SEQ ID NOs: 119-120, and SEQ ID NOs: 281-282) can be used in any of the various aspects of the invention.

In a further aspect, the present invention features a method of diagnosing a pathogenic infection in a mammal by detecting the presence of the nucleic acid or the protein of the invention in the mammal.

The invention further provides a method of determining whether a bacterium is pathogenic by detecting the presence of the nucleic acid or the protein of the invention in the bacteria. In all foregoing aspects of the invention, the nucleic acid and protein of the invention may be detected by means of a nucleic acid, a probe, or an antibody.

The invention also features a method of generating an antibody by (a) immunizing an animal with the protein of the invention or a fragment thereof; and (b) isolating an antibody that specifically binds such a protein or fragment. Optionally, the antibody inhibits the binding of a mammalian or plant protein to the protein of the invention.

The invention further features a method for identifying a virulence factor of a pathogen, involving the steps of: (a) contacting a factor from the pathogen with a protein having an amino acid sequence at least 25% identical to any one of the amino acid sequences of SEQ ID NOs: 269-277 under conditions that allow binding; and (b) detecting binding of this factor to the protein, thereby determining whether the factor is a virulence factor. Alternatively, the invention also provides a method for identifying a compound that inhibits virulence of a pathogen, including the steps of: (a) contacting a candidate compound, a factor from the pathogen, and a protein having an amino acid sequence at least 25% identical to any one of the amino acid sequences of SEQ ID NOs: 269-277 under conditions that allow binding; and (b) measuring the binding of the factor to the protein, such that a decrease in binding effected by the candidate compound indicates that the candidate compound inhibits the virulence of the pathogen. Preferably, the pathogen is Pseudomonas aeruginosa.

In yet another aspect, the invention features a method of diagnosing a Pseudomonas or Pseudomonas-related infection in a mammal by detecting binding of a sample from the mammal to a protein containing any one of the amino acid sequences of SEQ ID NOs: 269-277.

In yet another related aspect, the invention provides a method for delivering a molecule to the lungs of a mammal by administering a molecule bound to a protein of the invention to the mammal under conditions that allow the protein to target the molecule to the lungs of the mammal.

The present invention also provides a method for delivering a protein of interest to the lungs of a mammal by administering a fusion protein that contains a protein of the invention as well as a protein of interest to the mammal under conditions that allow the fusion protein to target the protein of interest to the lungs of the mammal.

By “isolated nucleic acid molecule” is meant a nucleic acid (e.g., a DNA) that is free of the genes which, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “polypeptide” is meant any chain of amino acids, regardless of length or post-translational modification (for example, glycosylation or phosphorylation).

By a “substantially pure polypeptide” is meant a polypeptide of the invention that has been separated from components which naturally accompany it. Typically, the polypeptide is substantially pure when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. A substantially pure polypeptide of the invention may be obtained, for example, by extraction from a natural source (for example, a pathogen); by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 25% identity to a reference amino acid sequence (for example, any one of the amino acid sequences described herein) or nucleic acid sequence (for example, any one of the nucleic acid sequences described herein). Preferably, such a sequence is at least 30%, 40%, 50%, 60%, 70%, more preferably 80%, 81%, 82%, 83%, 84%, 85% identical, and most preferably 90%, 92%, 94%, 95%, 96%, 97%, 98%, or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a DNA molecule encoding (as used herein) a polypeptide of the invention.

By “positioned for expression” is meant that the DNA molecule is positioned adjacent to a DNA sequence which directs transcription and translation of the sequence (i.e., facilitates the production of, for example, a recombinant polypeptide of the invention, or an RNA molecule).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “inhibiting a pathogen” is meant the ability of a candidate compound to decrease, suppress, attenuate, diminish, or arrest the development or progression of a pathogen-mediated disease or an infection in a eukaryotic host organism. Preferably, such inhibition decreases pathogenicity by at least 5%, more preferably by at least 25%, and most preferably by at least 50%, as compared to symptoms in the absence of the candidate compound in any appropriate pathogenicity assay (for example, those assays described herein). In one particular example, inhibition may be measured by monitoring pathogenic symptoms in a host organism exposed to a candidate compound or extract, a decrease in the level of symptoms relative to the level of pathogenic symptoms in a host organism not exposed to the compound indicating compound-mediated inhibition of the pathogen.

By “pathogenic virulence factor” is meant a cellular component (e.g., a protein such as a transcription factor, as well as the gene which encodes such a protein) without which the pathogen is incapable of causing disease or infection in a eukaryotic host organism.

By “antisense” is meant a nucleic acid, regardless of length, that is complementary to a coding strand or mRNA of the invention. In some embodiments, the antisense molecule inhibits the expression of only one nucleic acid, and in other embodiments, the antisense molecule inhibits the expression of more than one nucleic acid. Desirably, the antisense nucleic acid decreases the expression or biological activity of a nucleic acid or protein of the invention by at least 20, 40, 50, 60, 70, 80, 90, 95, or 100%. An antisense molecule can be introduced, e.g., to an individual cell or to whole animals, for example, it may be introduced systemically via the bloodstream. Desirably, a region of the antisense nucleic acid or the entire antisense nucleic acid is at least 70, 80, 90, 95, 98, or 100% complimentary to a coding sequence, regulatory region (5′ or 3′ untranslated region), or an mRNA of interest. Desirably, the region of complementarity includes at least 5, 10, 20, 30, 50, 75, 100, 200, 500, 1000, 2000, or 5000 nucleotides or includes all of the nucleotides in the antisense nucleic acid.

In some embodiments, the antisense molecule is less than 200, 150, 100, 75, 50, or 25 nucleotides in length. In other embodiments, the antisense molecule is less than 50,000; 10,000; 5,000; or 2,000 nucleotides in length. In certain embodiments, the antisense molecule is at least 200, 300, 500, 1000, or 5000 nucleotides in length. In some embodiments, the number of nucleotides in the antisense molecule is contained in one of the following ranges: 5-15 nucleotides, 16-20 nucleotides, 21-25 nucleotides, 26-35 nucleotides, 36-45 nucleotides, 46-60 nucleotides, 61-80 nucleotides, 81-100 nucleotides, 101-150 nucleotides, or 151-200 nucleotides, inclusive. In addition, the antisense molecule may contain a sequence that is less than a full-length sequence or may contain a full-length sequence.

By “double stranded RNA” is meant a nucleic acid containing a region of two or more nucleotides that are in a double stranded conformation. In various embodiments, the double stranded RNA consists entirely of ribonucleotides or consists of a mixture of ribonucleotides and deoxynucleotides. The double stranded RNA may be a single molecule with a region of self-complementary such that nucleotides in one segment of the molecule base pair with nucleotides in another segment of the molecule. Alternatively, the double stranded RNA may include two different strands that have a region of complementarity to each other. Desirably, the regions of complementarity are at least 70, 80, 90, 95, 98, or 100% complimentary. Desirably, the region of the double stranded RNA that is present in a double stranded conformation includes at least 5, 10, 20, 30, 50, 75, 100, 200, 500, 1000, 2000 or 5000 nucleotides or includes all of the nucleotides in the double stranded RNA. Desirable double stranded RNA molecules have a strand or region that is at least 70, 80, 90, 95, 98, or 100% identical to a coding region or a regulatory sequence (e.g., a transcription factor binding site, a promoter, or a 5′ or 3′ untranslated region) of a nucleic acid of the invention. In some embodiments, the double stranded RNA is less than 200, 150, 100, 75, 50, or 25 nucleotides in length. In other embodiments, the double stranded RNA is less than 50,000; 10,000; 5,000; or 2,000 nucleotides in length. In certain embodiments, the double stranded RNA is at least 200, 300, 500, 1000, or 5000 nucleotides in length. In some embodiments, the number of nucleotides in the double stranded RNA is contained in one of the following ranges: 5-15 nucleotides, 16-20 nucleotides, 21-25 nucleotides, 26-35 nucleotides, 36-45 nucleotides, 46-60 nucleotides, 61-80 nucleotides, 81-100 nucleotides, 101-150 nucleotides, or 151-200 nucleotides, inclusive. In addition, the double stranded RNA may contain a sequence that is less than a full-length sequence or may contain a full-length sequence.

In some embodiments, the double stranded RNA molecule inhibits the expression of only one nucleic acid, and in other embodiments, the double stranded RNA molecule inhibits the expression of more than one nucleic acid. Desirably, the nucleic acid decreases the expression or biological activity of a nucleic acid or protein of the invention by at least 20, 40, 50, 60, 70, 80, 90, 95, or 100%. A double stranded RNA can be introduced, e.g., to an individual cell or to whole animals, for example, it may be introduced systemically via the bloodstream.

In various embodiments, the double stranded RNA or antisense molecule includes one or more modified nucleotides in which the 2′ position in the sugar contains a halogen (such as fluorine group) or contains an alkoxy group (such as a methoxy group) which increases the half-life of the double stranded RNA or antisense molecule in vitro or in vivo compared to the corresponding double stranded RNA or antisense molecule in which the corresponding 2′ position contains a hydrogen or an hydroxyl group. In yet other embodiments, the double stranded RNA or antisense molecule includes one or more linkages between adjacent nucleotides other than a naturally-occurring phosphodiester linkage. Examples of such linkages include phosphoramide, phosphorothioate, and phosphorodithioate linkages.

The invention provides a number of targets that are useful for the development of drugs that specifically block the pathogenicity of a microbe, for example, Pseudomonas aeruginosa PA14. In addition, the methods of the invention provide a facile means to identify compounds that are safe for use in eukaryotic host organisms (i.e., compounds which do not adversely affect the normal development and physiology of the organism), and efficacious against pathogenic microbes (i.e., by suppressing the virulence of a pathogen). In addition, the methods of the invention provide a route for analyzing virtually any number of compounds for an anti-virulence effect with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIGS. 1A-AB show a polynucleotide sequence including 10,848 base pairs of PAPI-2 (SEQ ID NO: 1), the small pathogenicity island of Pseudomonas aeruginosa PA14 described herein.

FIGS. 2A-2P show a polynucleotide sequence including 84,830 base pairs of PAPI-1 (SEQ ID NO: 2), the large pathogenicity island of Pseudomonas aeruginosa PA14 described herein.

FIG. 3 is the polynucleotide sequence (SEQ ID NO: 3) and the translated amino acid sequence (SEQ ID NO: 127) of ORF RL024.

FIG. 4 is the polynucleotide sequence (SEQ ID NO: 4) and the translated amino acid sequence (SEQ ID NO: 128) of ORF RL025.

FIG. 5 is the polynucleotide sequence (SEQ ID NO: 5) and the translated amino acid sequence (SEQ ID NO: 129) of ORF RL026.

FIG. 6 is the polynucleotide sequence (SEQ ID NO: 6) and the translated amino acid sequence (SEQ ID NO: 130) of ORF RL027.

FIG. 7 is the polynucleotide sequence (SEQ ID NO: 7) and the translated amino acid sequence (SEQ ID NO: 131) of ORF RL028.

FIG. 8 is the polynucleotide sequence (SEQ ID NO: 8) and the translated amino acid sequence (SEQ ID NO: 132) of ORF RL029.

FIG. 9 is the polynucleotide sequence (SEQ ID NO: 9) and the translated amino acid sequence (SEQ ID NO: 133) of ORF RL030.

FIG. 10 is the polynucleotide sequence (SEQ ID NO: 10) and the translated amino acid sequence (SEQ ID NO: 134) of ORF RL031.

FIG. 11 is the polynucleotide sequence (SEQ ID NO: 11) and the translated amino acid sequence (SEQ ID NO: 135) of ORF RL032.

FIG. 12 is the polynucleotide sequence (SEQ ID NO: 12) and the translated amino acid sequence (SEQ ID NO: 136) of ORF RL033.

FIG. 13 is the polynucleotide sequence (SEQ ID NO: 13) and the translated amino acid sequence (SEQ ID NO: 137) of ORF RL034.

FIG. 14 is the polynucleotide sequence (SEQ ID NO: 14) and the translated amino acid sequence (SEQ ID NO: 138) of ORF RL035.

FIG. 15 is the polynucleotide sequence (SEQ ID NO: 15) and the translated amino acid sequence (SEQ ID NO: 139) of ORF RL036.

FIG. 16 is the polynucleotide sequence (SEQ ID NO: 16) and the translated amino acid sequence (SEQ ID NO: 140) of ORF RL037.

FIG. 17 is the polynucleotide sequence (SEQ ID NO: 17) and the translated amino acid sequence (SEQ ID NO: 141) of ORF RL038.

FIG. 18 is the polynucleotide sequence (SEQ ID NO: 18) and the translated amino acid sequence (SEQ ID NO: 142) of ORF RL039.

FIG. 19 is the polynucleotide sequence (SEQ ID NO: 19) and the translated amino acid sequence (SEQ ID NO: 143) of ORF RL040.

FIG. 20 is the polynucleotide sequence (SEQ ID NO: 20) and the translated amino acid sequence (SEQ ID NO: 144) of ORF RL041.

FIG. 21 is the polynucleotide sequence (SEQ ID NO: 21) and the translated amino acid sequence (SEQ ID NO: 145) of ORF RL042.

FIG. 22 is the polynucleotide sequence (SEQ ID NO: 22) and the translated amino acid sequence (SEQ ID NO: 146) of ORF RL043.

FIG. 23 is the polynucleotide sequence (SEQ ID NO: 23) and the translated amino acid sequence (SEQ ID NO: 147) of ORF RL044.

FIG. 24 is the polynucleotide sequence (SEQ ID NO: 24) of ORF RL045.

FIG. 25 is the polynucleotide sequence (SEQ ID NO: 25) and the translated amino acid sequence (SEQ ID NO: 148) of ORF RL046.

FIG. 26 is the polynucleotide sequence (SEQ ID NO: 26) and the translated amino acid sequence (SEQ ID NO: 149) of ORF RL047.

FIG. 27 is the polynucleotide sequence (SEQ ID NO: 27) and the translated amino acid sequence (SEQ ID NO: 150) of ORF RL048.

FIG. 28 is the polynucleotide sequence (SEQ ID NO: 28) and the translated amino acid sequence (SEQ ID NO: 151) of ORF RL049.

FIG. 29 is the polynucleotide sequence (SEQ ID NO: 29) and the translated amino acid sequence (SEQ ID NO: 152) of ORF RL050.

FIGS. 30A-30V show the polynucleotide sequences (SEQ ID NOs: 30-94) and the translated amino acid sequences (SEQ ID NOs: 153-215) of ORF RL051 to ORF RL115.

FIGS. 31A-31E shows the polynucleotide sequences (SEQ ID NOs: 95-109 and SEQ ID NO: 282) and the translated amino acid sequences (SEQ ID NOs: 216-229) of RS01 to RS15.

FIG. 32 is a table showing the nucleotide homology between regions of PAPI-1, the big island of pathogenicity (84, 830 bps) and other virulence factors.

FIG. 33 is a table showing the nucleotide homology between regions of PAPI-2, the small island of pathogenicity (10, 848 bps) and other virulence factors.

FIGS. 34A-34G represent a table showing the nucleotide homology between regions belonging to the various ORFs of the big pathogenicity island and other known proteins, including virulence factors.

FIG. 35 is an alignment of clone 2 with Homo sapiens mRNA EST DKFZp566K094_r1 (from clone DKFZp566) (SEQ ID NOs: 230-232).

FIG. 36 is an alignment of clone 8 with EST01285 subtracted hippocampus (Stratagene, cat. #936205) (SEQ ID NOs: 233-235).

FIG. 37 is an alignment of clone 47 with fibrillin 1 precursor (SEQ ID NOs: 236-238).

FIG. 38 is an alignment of clone 56 with emilin precursor (SEQ ID NOs: 239-244).

FIG. 39 is an alignment of clone 59 with a fragment of pironly|A35763|A35763 collagen alpha 2 chain—sea urchin (Paracentrotus lividus) (SEQ ID NOs: 245-250).

FIG. 40 is an alignment of clone 60/63 with transcobalamin II precursor (SEQ ID NOs: 251-259).

FIG. 41 is an alignment of clone 65 with human fibulin-1 precursor (SEQ ID NOs: 260-262).

FIG. 42 is an alignment of clone 80 with trembl|AF045447|AF045447_{—}1 deleted in pancreatic carcinoma (DPC4) (SEQ ID NOs: 263-265).

FIG. 43 is an alignment of clone 86 with the cell surface protein Notch2 (SEQ ID NOs: 266-268).

FIG. 44 is the polynucleotide sequence (SEQ ID NO: 109) and the translated amino acid sequence (SEQ ID NO: 269) of a human lung nucleic acid molecule.

FIG. 45 is the polynucleotide sequence (SEQ ID NO: 110) and the translated amino acid sequence (SEQ ID NO: 270) of a human lung nucleic acid molecule.

FIG. 46 is the polynucleotide sequence (SEQ ID NO: 111) and the translated amino acid sequence (SEQ ID NO: 271) of a human lung nucleic acid molecule.

FIG. 47 is the polynucleotide sequence (SEQ ID NO: 112) and the translated amino acid sequence (SEQ ID NO: 272) of a human lung nucleic acid molecule.

FIG. 48 is the polynucleotide sequence (SEQ ID NO: 113) and the translated amino acid sequence (SEQ ID NO: 273) of a human lung nucleic acid molecule.

FIG. 49 is the polynucleotide sequence (SEQ ID NO: 114) and the translated amino acid sequence (SEQ ID NO: 274) of a human lung nucleic acid molecule.

FIG. 50 is the polynucleotide sequence (SEQ ID NO: 115) and the translated amino acid sequence (SEQ ID NO: 275) of a human lung nucleic acid molecule.

FIG. 51 is the polynucleotide sequence (SEQ ID NO: 116) and the translated amino acid sequence (SEQ ID NO: 276) of a human lung nucleic acid molecule.

FIG. 52 is the polynucleotide sequence (SEQ ID NOs: 117-118) and the translated amino acid sequence (SEQ ID NO: 277) of a human lung nucleic acid molecule.

FIG. 53 is a table that lists P. aeruginosa strains containing a nucleic acid that hybridized to a nucleic acid probe of the invention (i.e., a probe containing a region of a nucleic acid of the invention).

FIG. 54 is the translated amino acid sequence of ORF7 (SEQ ID NO: 278).

FIG. 55 is the polynucleotide sequence of ORF7 (SEQ ID NO: 119).

FIG. 56 is the translated amino acid sequence of clpB (SEQ ID NO: 279)

FIG. 57 is the polynucleotide sequence of clpB (SEQ ID NO: 120).

FIG. 58 is a table showing the nucleotide homology between regions belonging to the various ORFs of the small pathogenicity island and other known proteins, including virulence factors.

FIGS. 59A and 59B are schematic diagrams showing the alignment of the PA14 and PAO1 genomes.

FIGS. 60A and 60B are schematic diagrams showing the organization of the PAPI-1 and PAPI-2 elements, respectively. The boxes with arrows represent individual ORFs and their transcriptional orientations. Empty boxes represent pseudogenes, triangles correspond to tRNA genes, and the marked vertical line corresponds to the PAPI-1 and PAPI-2 attR. The numbered lines represent size (kb), and the coincident rectangles and single or double-headed arrows on the line respectively correspond to direct repeats (DR1-5), inverted repeat (IR), and IS sequences. ORF pattern corresponds and the bacterial species that it is most related to. Also indicated is the predicted protein function of ORFs, such as toxin/secreted factor (A), adhesion/protein secretion (B), regulation (C), DNA recombination/replication (D), hypothetical (E), and unclassified (F). Pathogenesis-related ORFs are indicated by shadowing (double arrow). Functions of gene clusters are marked and correspond to ORFs above the notations. The regions marked with a (*) show the homology between PAPI-1 and PAPI-2.

FIG. 61 is a diagram showing the presence of PAPI-1 in P. aeruginosa clinical isolates. The upper line represents the PAPI-1 coordinates (kb). The arrowheads indicate the position of the direct repeats (DR). The black rectangles correspond to the probes used for hybridization. (+) denotes positive hybridization; (N) denotes experiment not done. All strains giving positive hybridization are shown.

FIGS. 62A and 62B show the cosmid clones containing genetic inserts from PAPI-1 and PAPI-2, respectively.

FIG. 63 is a table showing bacterial strains bearing mutations in PAPI ORFs and their effect on mouse mortality and growth in Arabidopsis leaf.

FIG. 64 is a table showing the characteristics of direct and inverted repeat sequences in PAPI-1.

FIG. 65 is a table showing the IS sequences in PAPI-1 and PAPI-2 and the corresponding IS families.

FIG. 66 is a table showing the correspondence of the type IV B pilus/secretion system in PAPI-1 to the type II secretion systems of P. aeruginosa PA01.

FIG. 67 represents the amino acid sequence of ORF7 showing the additional 14 amino acids (SEQ ID NO: 280). Also shown is the corresponding nucleic acid sequence (SEQ ID NO: 281).

FIG. 68 represents the regulatory region of ORF7 (SEQ ID NO: 121) showing the two putative transcription start sites originating from the region inside PAPI-1. The arrows indicate the transcription start sites determined by primer extension experiments, with their position in relation to the translational start site, which is the boxed TTG. The −10 and −35 predicted regions of each promoter are shown in a shadowed box or empty box. Capital letters within the box indicate that the specific nucleotide is present in the consensus sequence of σ⁷⁰ dependent promoters. Underlined sequences correspond to tRNA genes codified by the opposite DNA strand. The sequence upstream of the highlighted “T” is not present in the PAO1 genome, indicating the beginning of the PA14 large pathogenicity island PAPI-1.

FIG. 69 is a photograph of an agarose gel electrophoresis of the PCR products obtained using as a template the genomic DNA of the strains indicated. M corresponds to the size marker.

FIGS. 70A-70B represent multiple sequence alignments (SEQ ID NOs: 122-126) of all PCR products using the Clustal W software.

### DETAILED DESCRIPTION

In general, the methods and compositions featured in the present invention are based on our discovery of pathogenicity islands harboring novel plant and animal virulence genes.

The versatile and ubiquitous bacterium Pseudomonas aeruginosa is the quintessential opportunistic pathogen, as it can infect a broad range of hosts, from amoeba to humans (Pukatzki et al., Proc Nail Acad Sci USA (2002) 99:3159-3164, and Rahme et al., Proc Natl Acad Sci USA (2000) 97:8815-8821), where it is found associated with severe burns, cystic fibrosis (CF), AIDS, or cancer (Govan et al., Microbiol. Rev. (1996) 60:539-574, and Bodey et al., Rev. Infect. Dis. (1983) 5:279-313). This pathogen produces an arsenal of virulence factors (Lyczak et al., Microbes Infect. (2000) 2:1051-1060) and displays a remarkable range of virulence, from weakly virulent isolates, to isolates that infect just a few organisms, to very broad spectrum isolates, exemplified by the clinical isolate PA14 (Rahme et al., Proc Natl Acad Sci USA (2000) 97:8815-21, Lau et al, Infect Immun (2003) 71:4059-4066 and Rahme et al., Science (1995) 268:1899-902.). Until now, the genomic basis of the promiscuity underlying the mechanisms of pathogenesis and defense of P. aeruginosa, as well as the origin, evolution, and utilization of such mechanisms by other infectious microorganisms has remained elusive.

Bacterial genomes are arranged in blocks of core sequences and genomic islands (Hacker et al., Annu Rev Microbiol (2000) 54:641-79, Parkhill et al., Nature (2001) 413:848-852, and Welch et al., Proc Natl Acad Sci USA (2002) 99:17020-17024). Genomic islands can greatly differ in their G+C content and can encode a variety of accessory activities that underlie specializations such as symbiotic and pathogenesis functions. Different genes carried by a single island often have diverse origins and blocks are built piecemeal through insertion and deletion events. Because genomic islands are typically acquired and exchanged by lateral gene transfer and are found in widely divergent species, it is often difficult to ascribe their initial origins. Pathogenicity islands, in particular, are specialized genomic islands that encode virulence factors. Their recent characterization in a wide range of pathogenic bacteria has led to the identification of novel virulence factors (e.g., adhesions, toxins, invasions, protein secretion systems, and iron uptake) used by these species to infect their respective hosts (Parkhill et al., Nature (2001) 413:848-852, Parkhill et al., Nature (2001) 413:523-527, Perna et al., Nature (2001) 409:529-533, da Silva et al., Nature (2002) 417:459-463, and Censini et al., Proc. Natl. Acad. Sci. U.S.A. (1996) 93:14648-14653.). Usually pathogenicity islands encompass large genomic regions (e.g., 10-200 kilobases) that are present on the genomes of pathogenic strains but absent from the genomes of nonpathogenic strains.

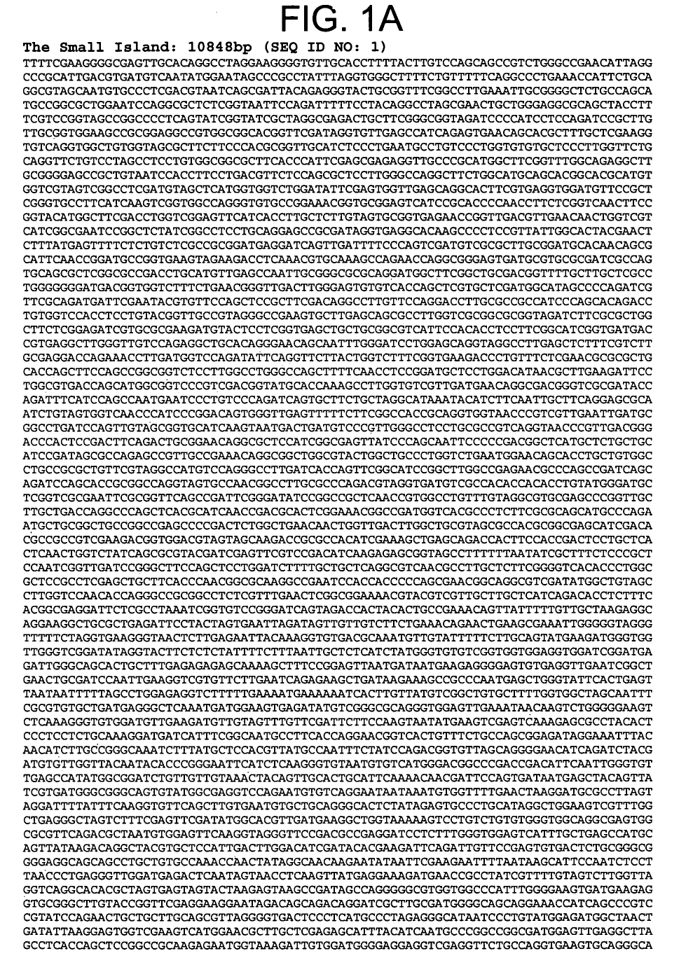

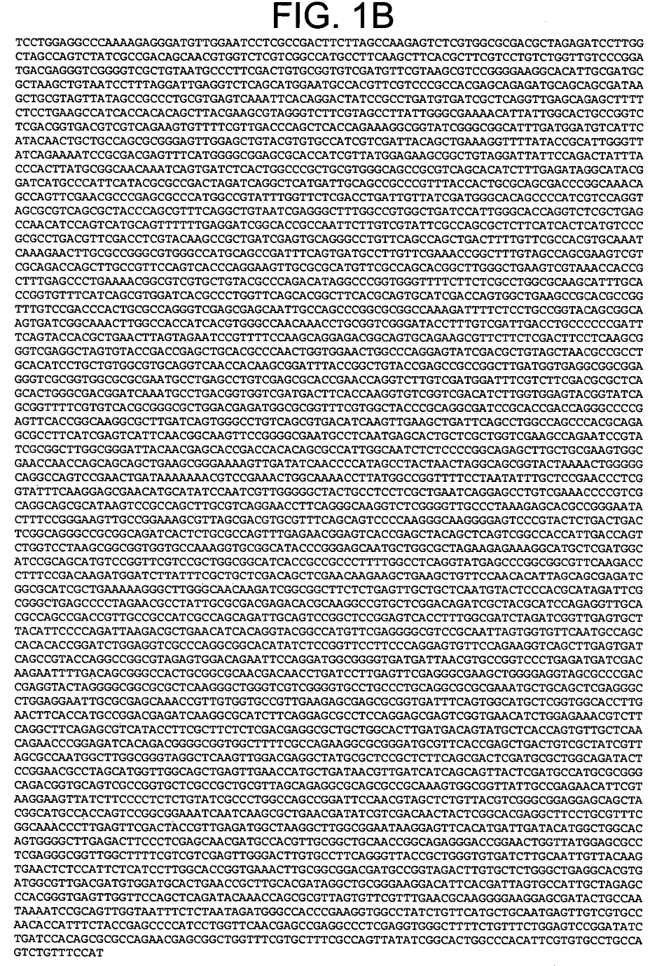

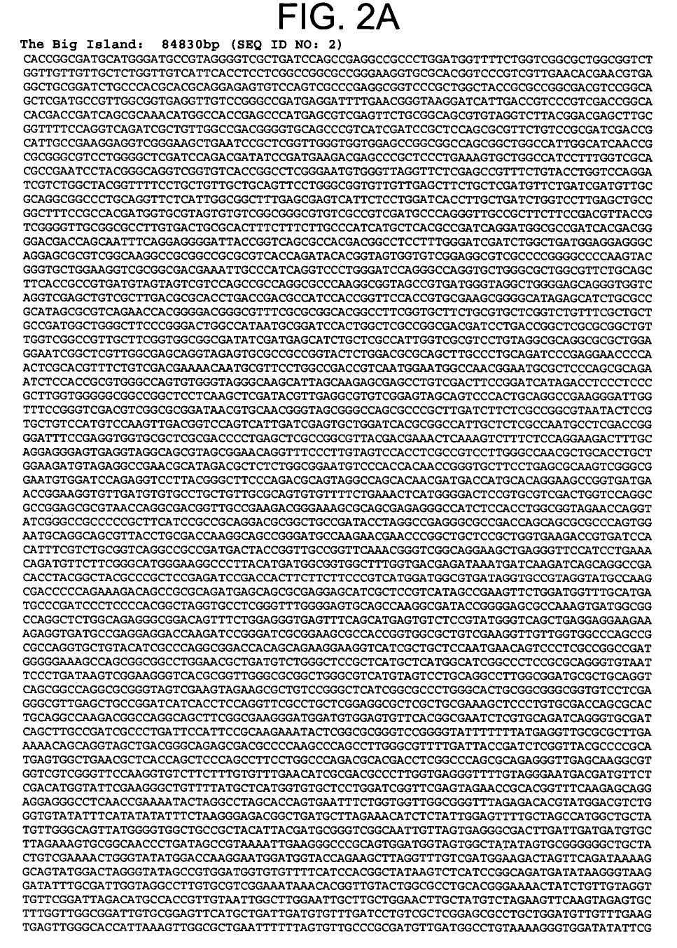

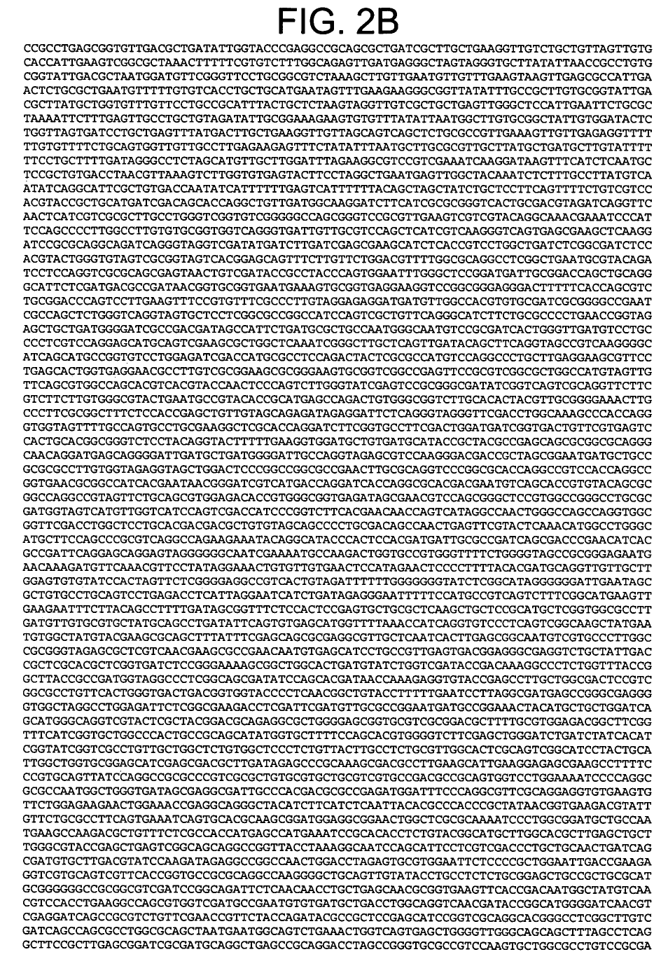

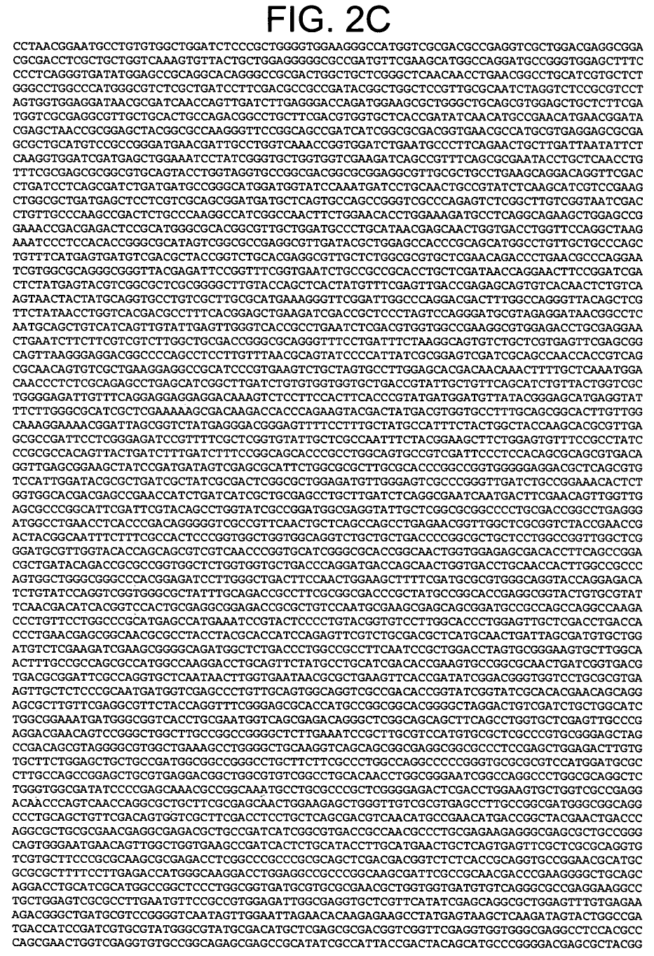

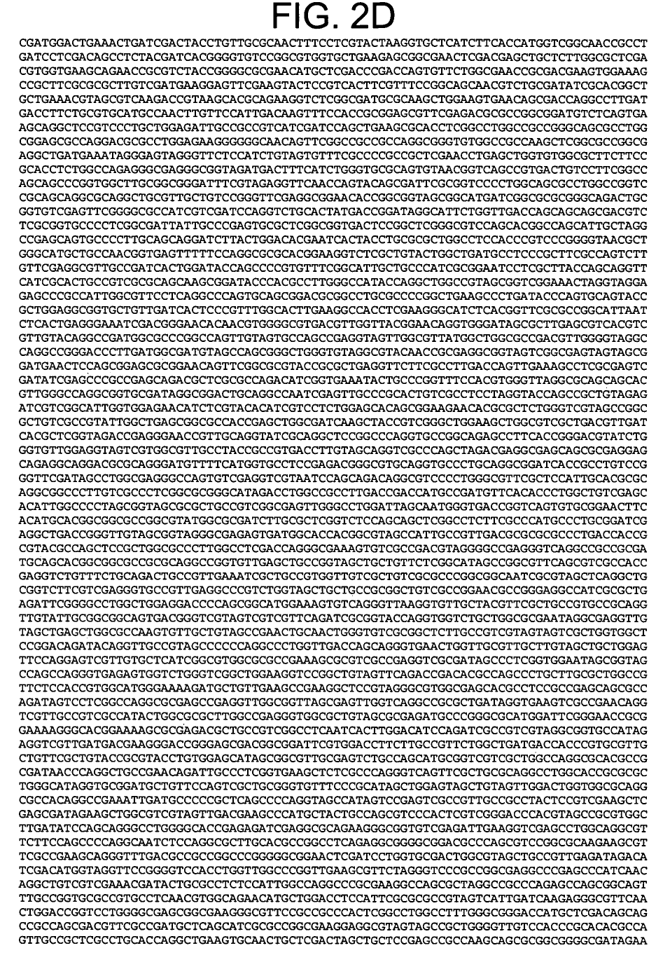

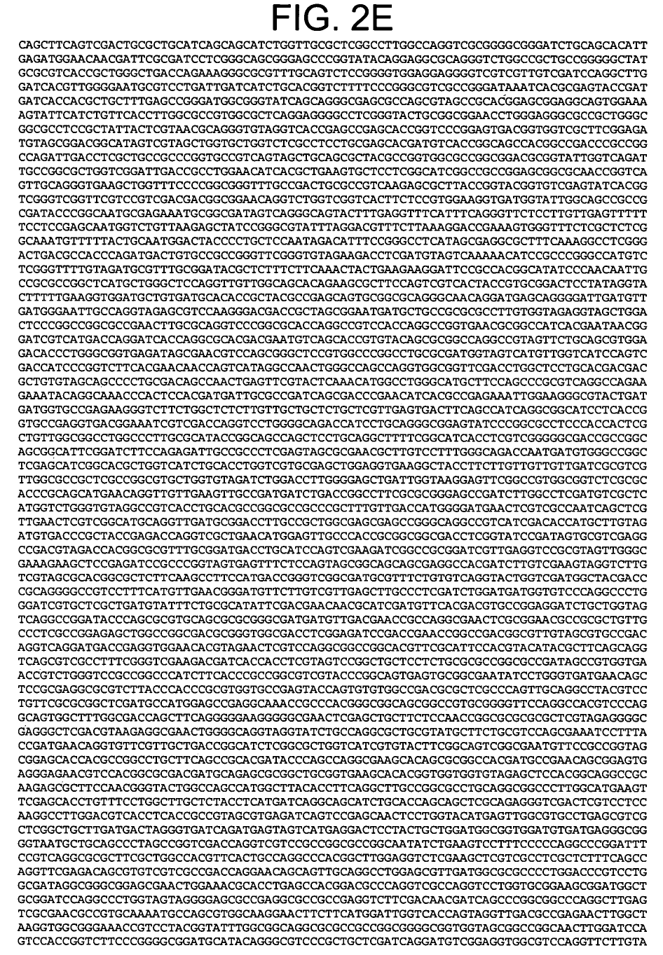

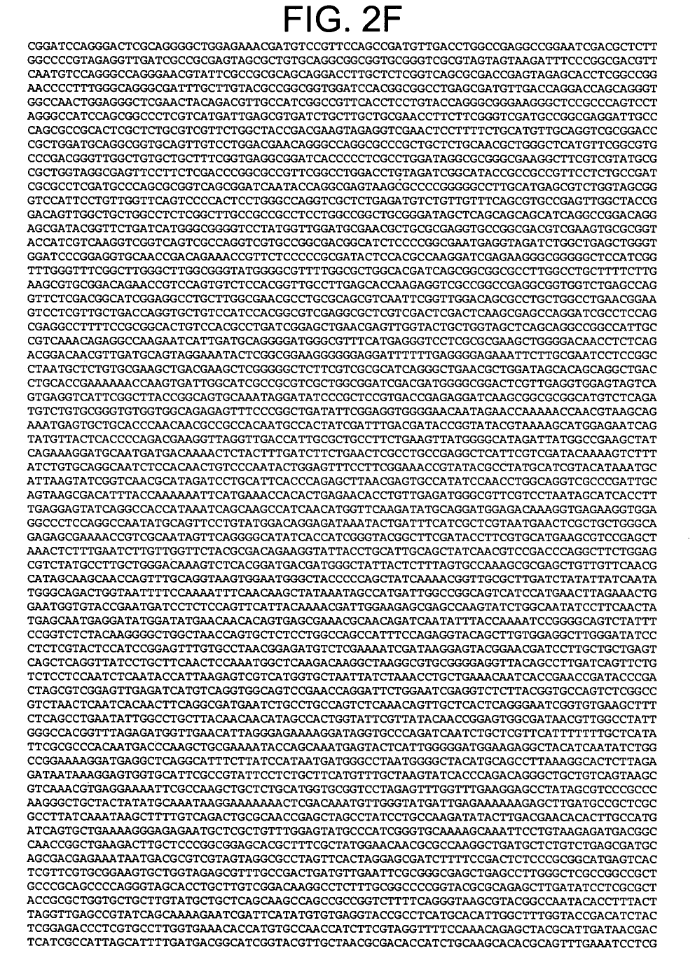

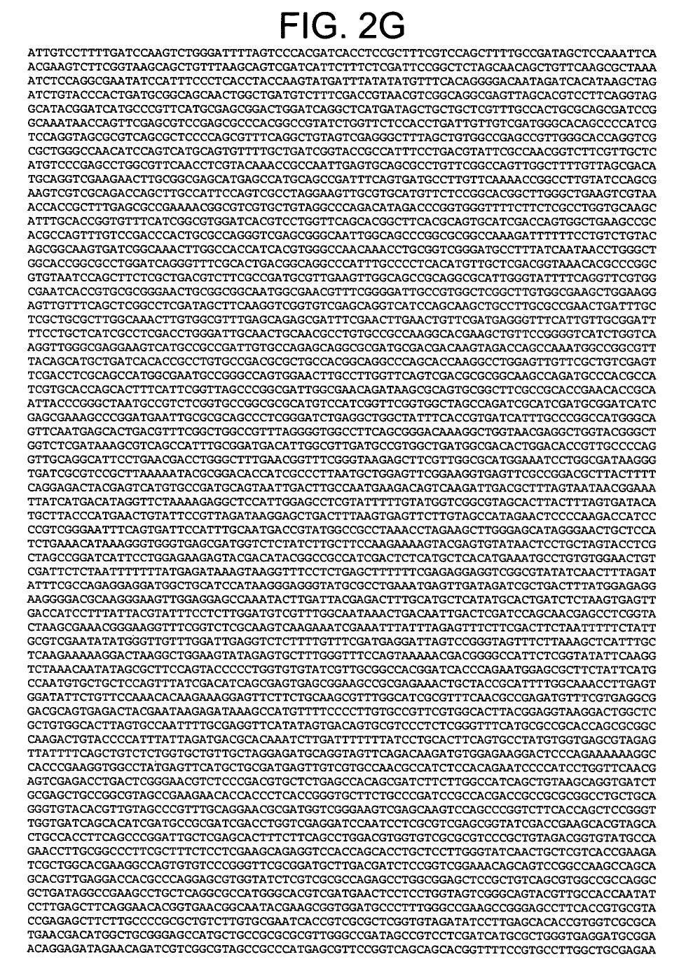

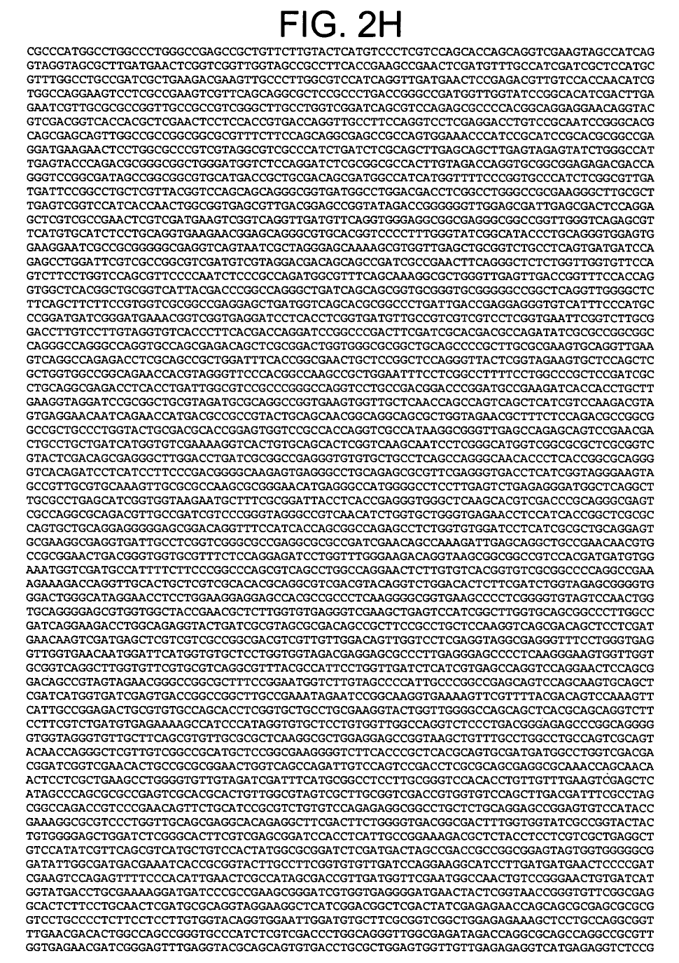

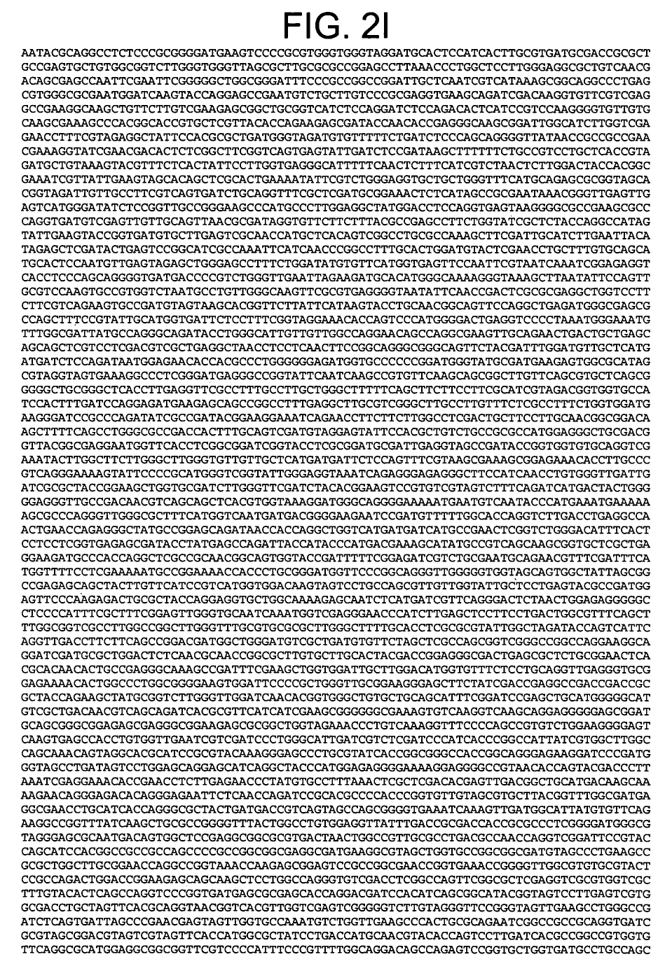

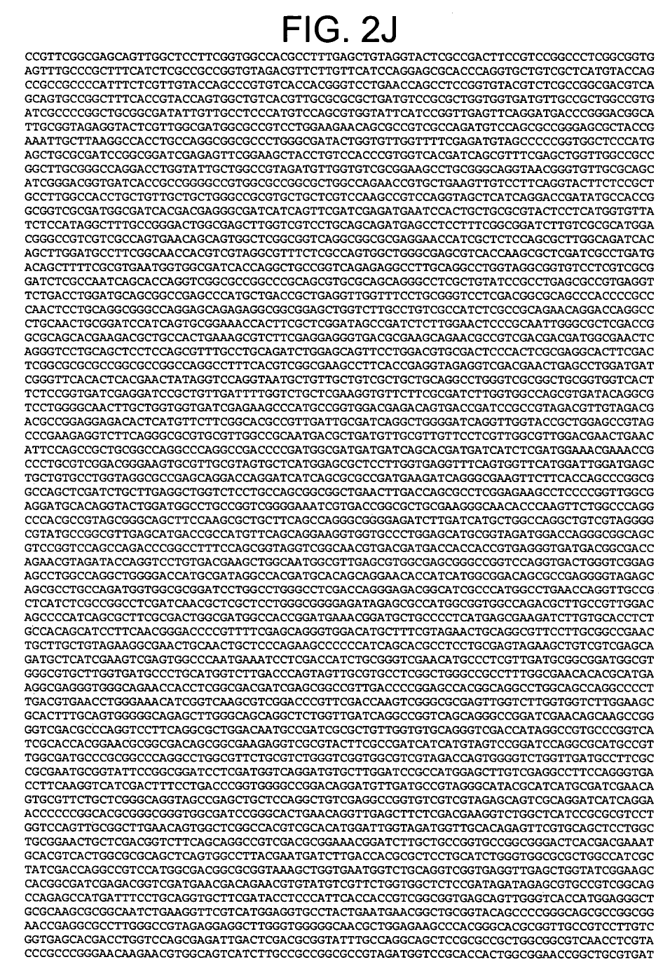

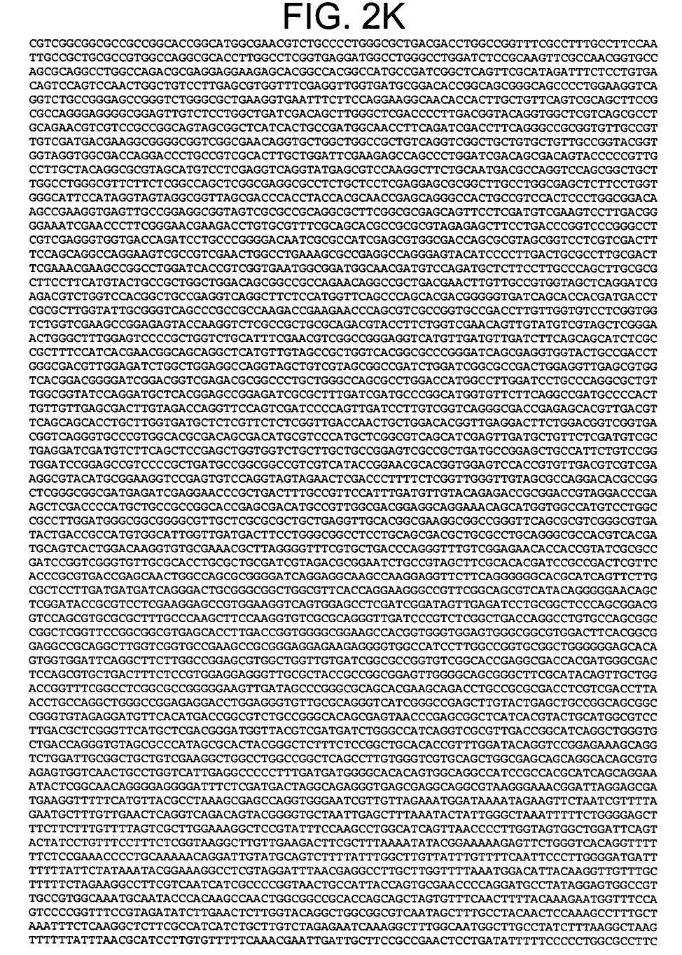

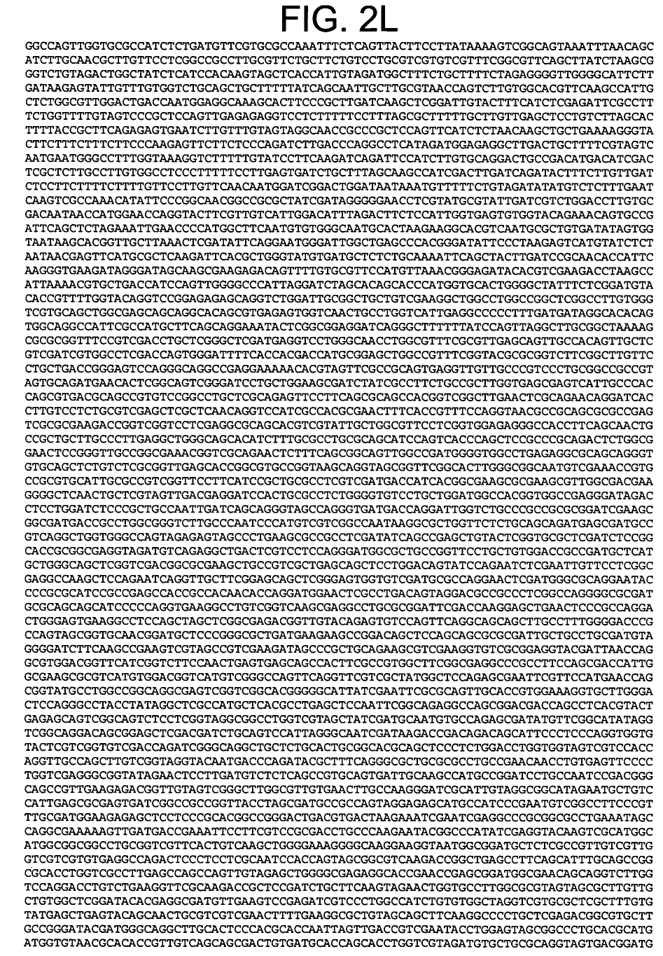

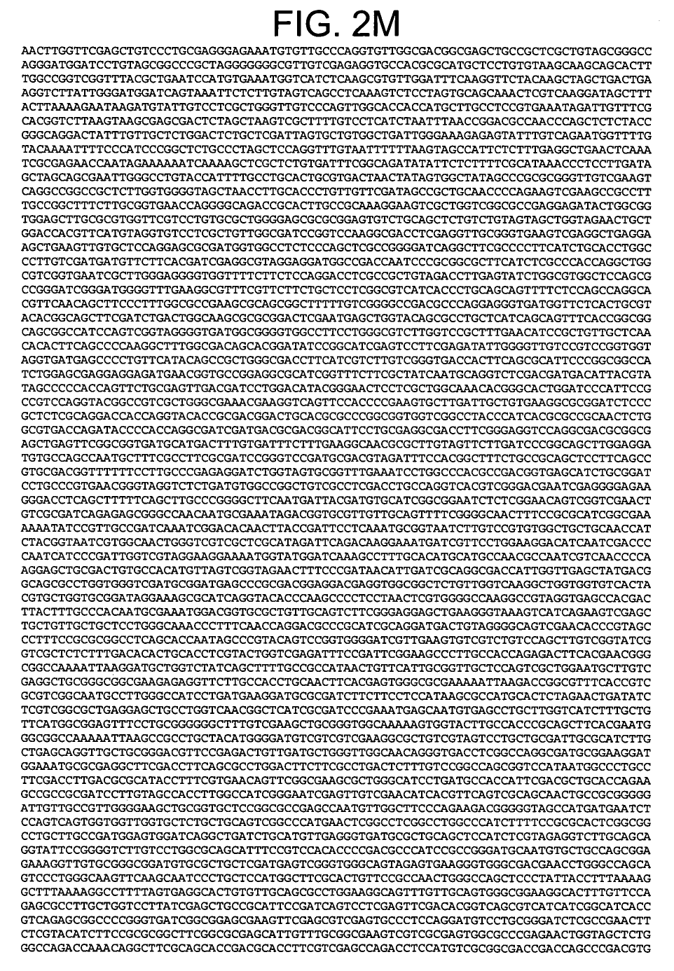

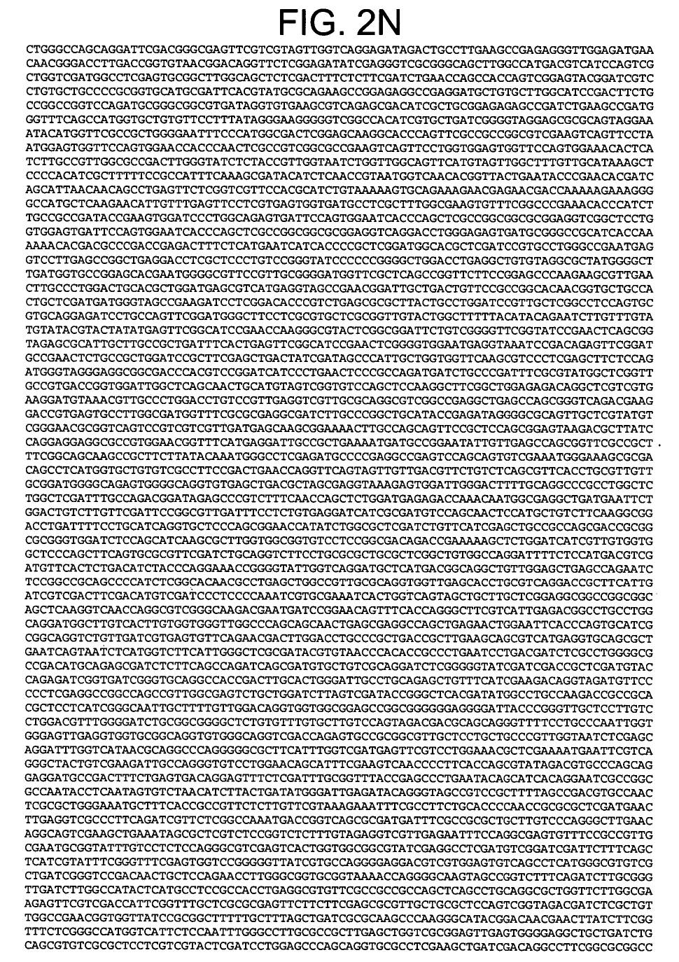

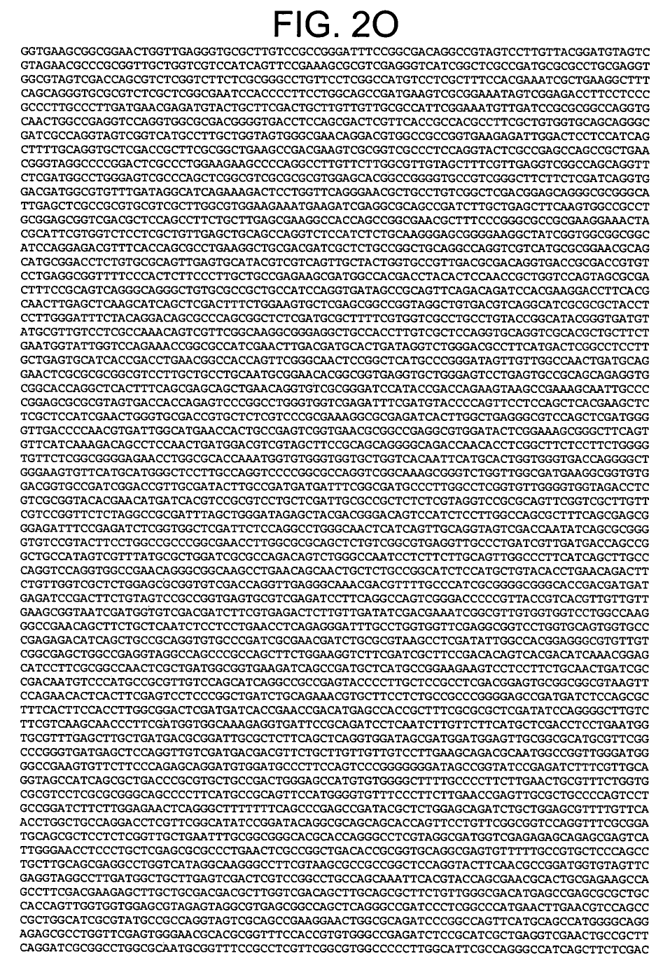

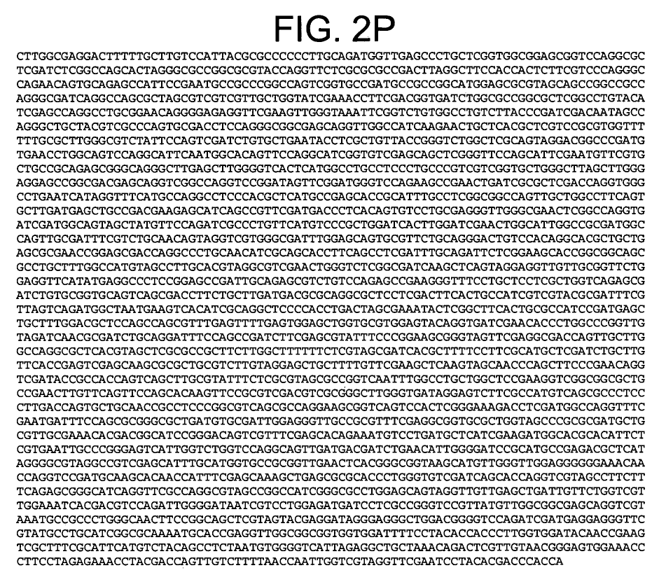

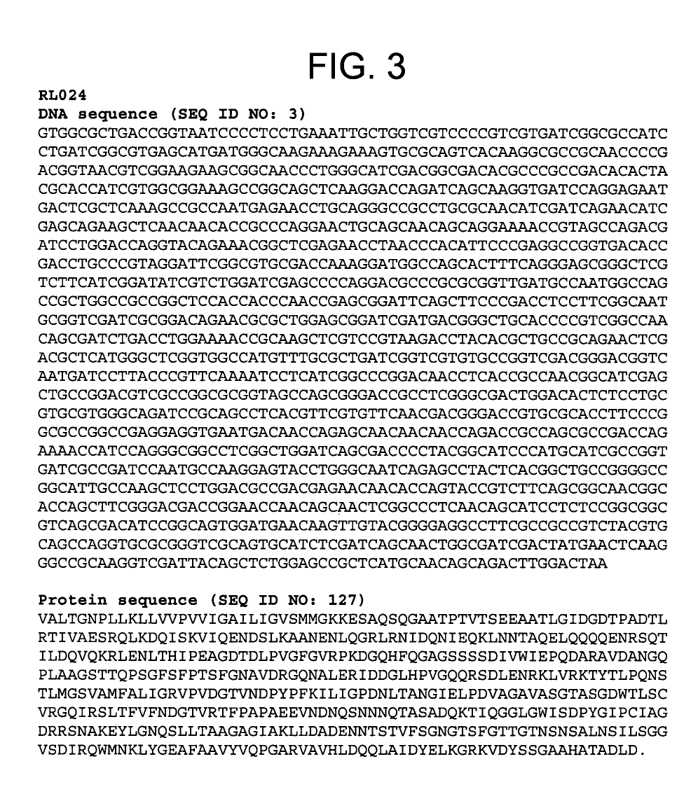

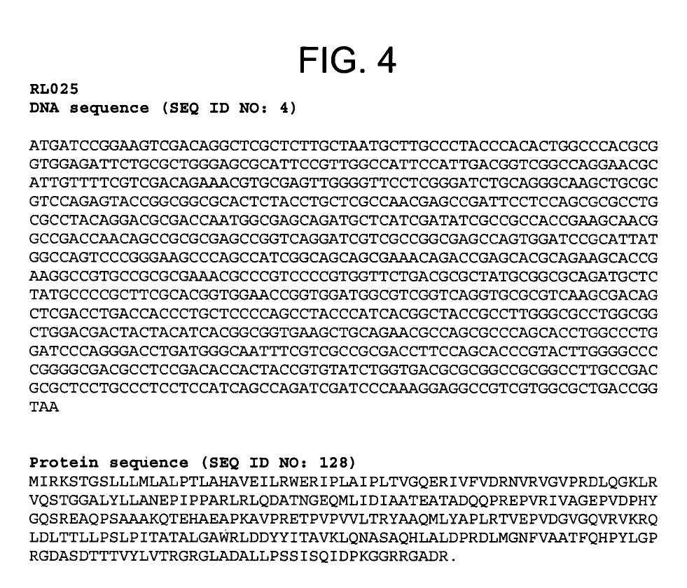

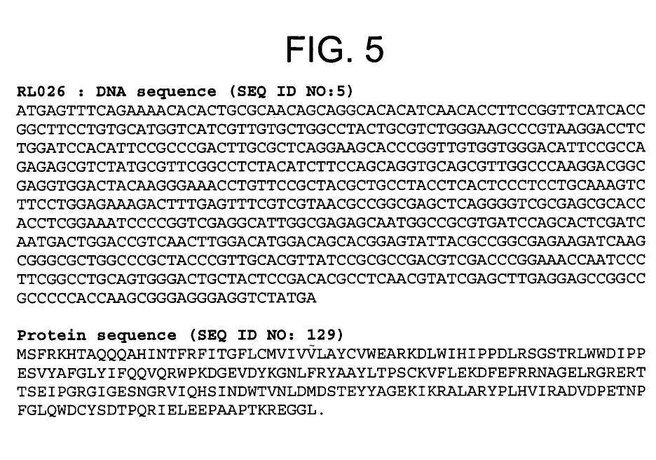

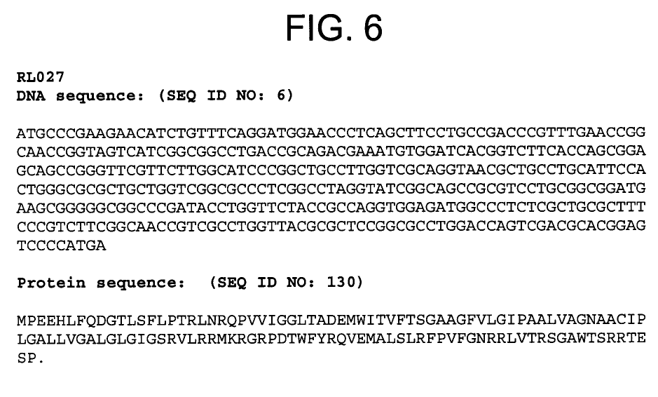

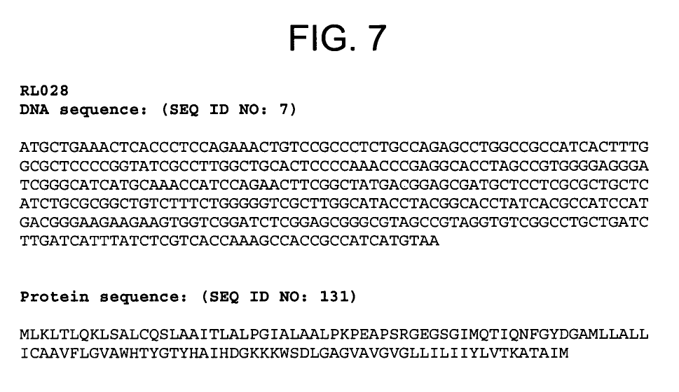

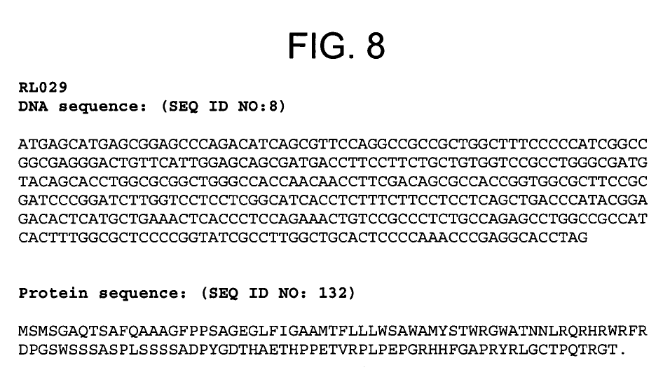

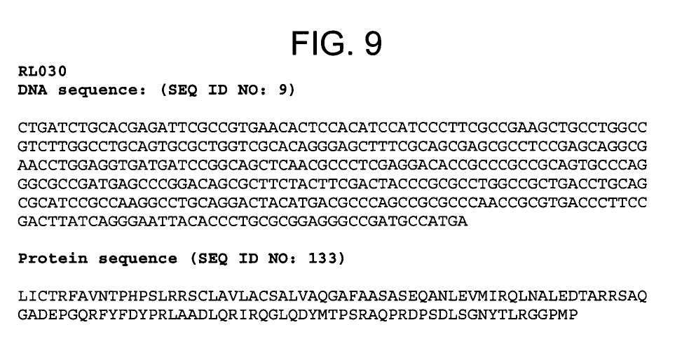

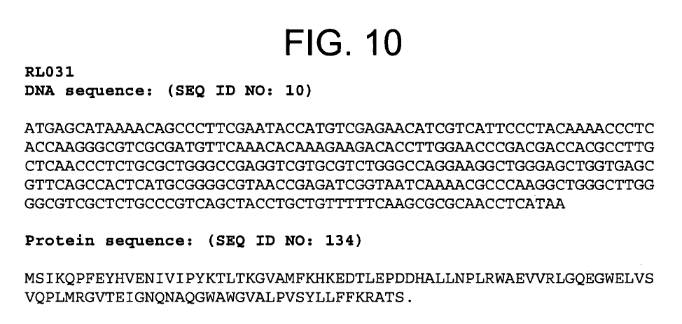

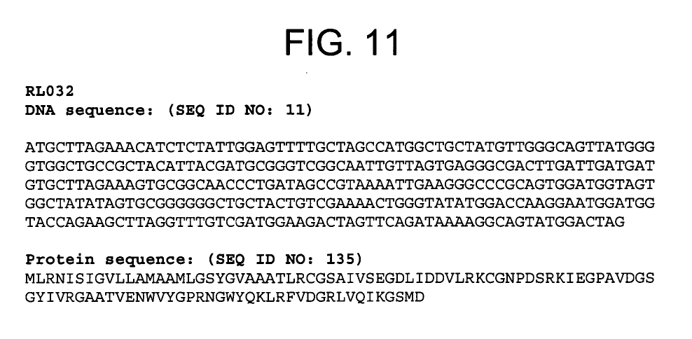

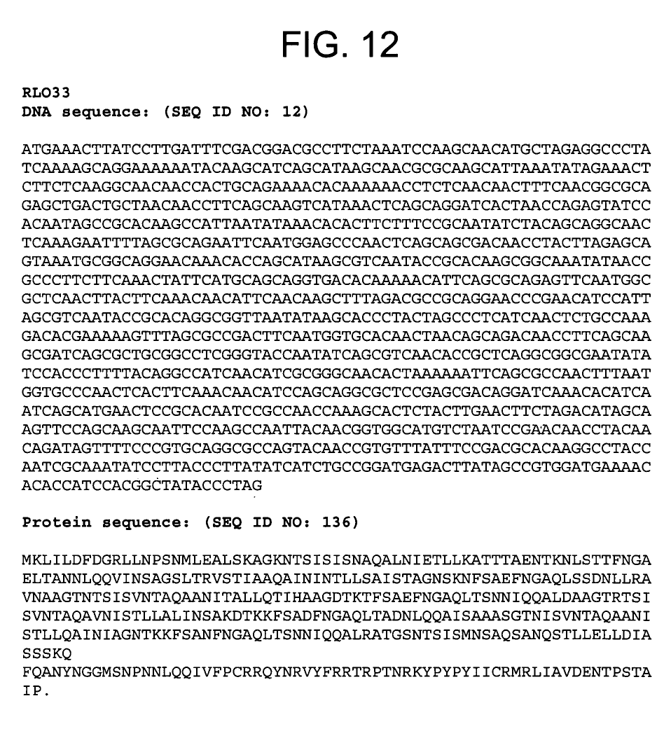

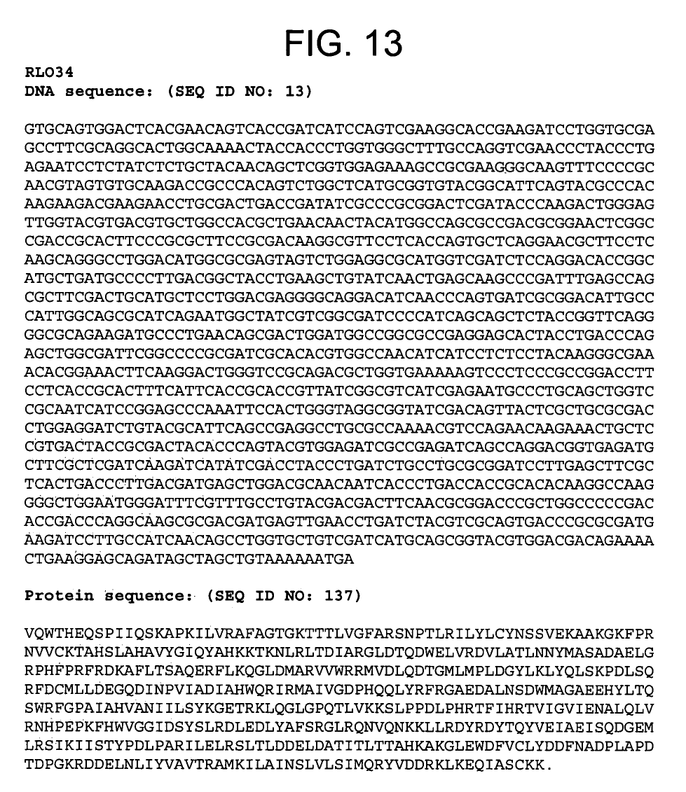

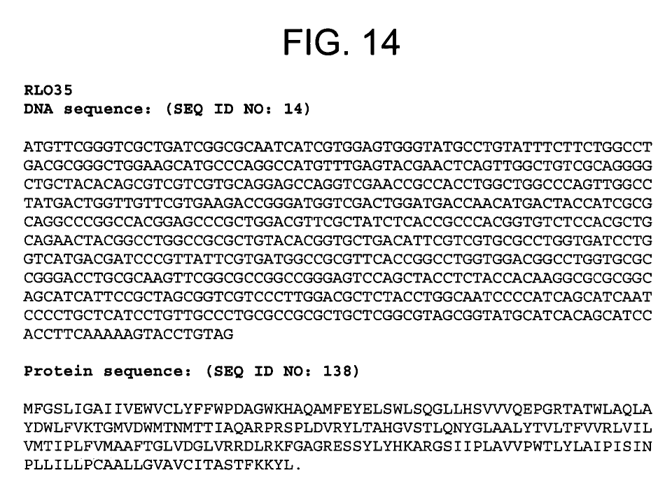

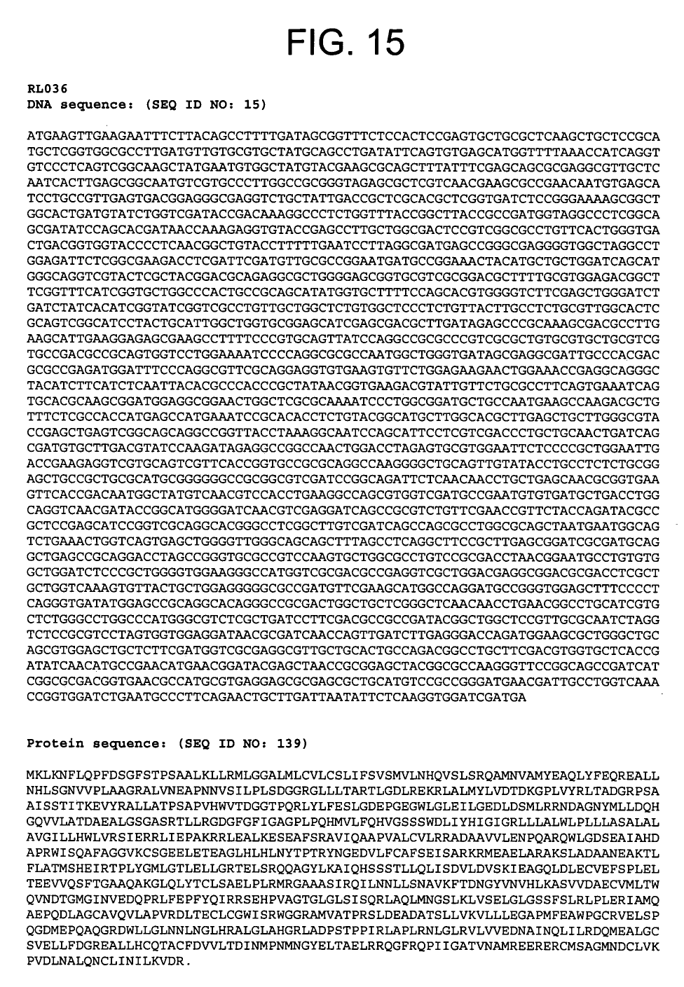

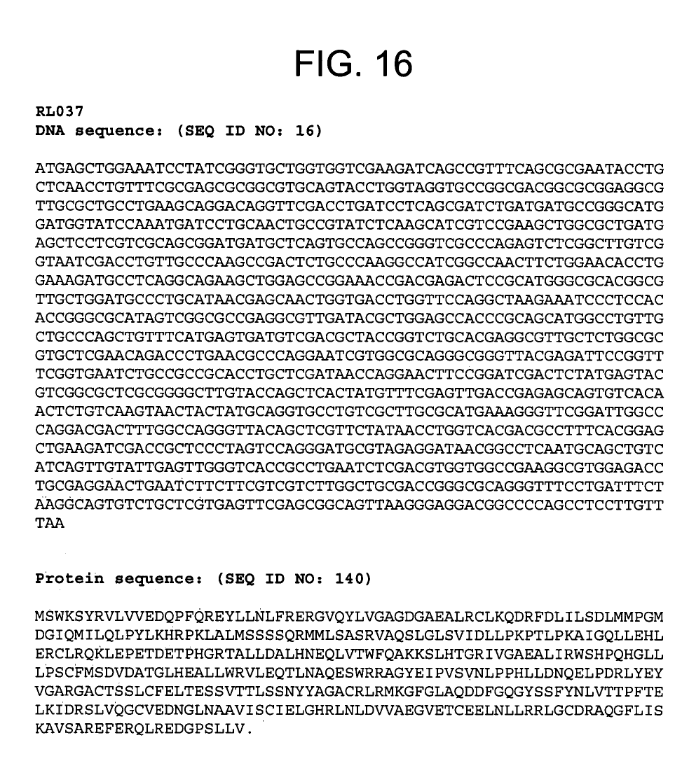

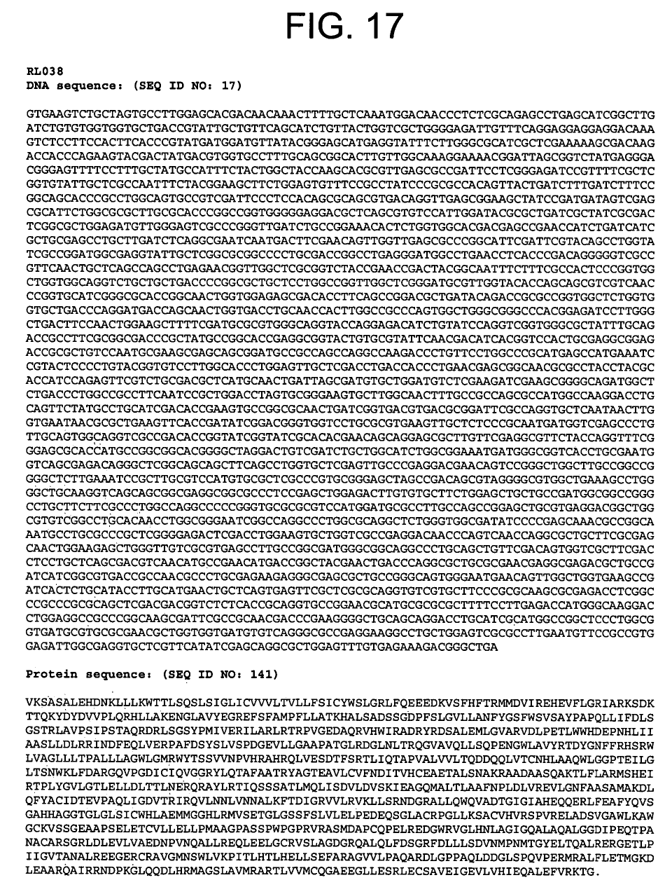

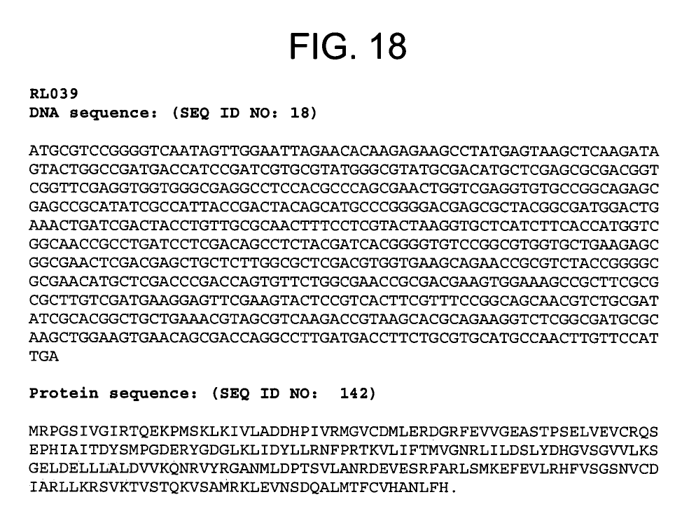

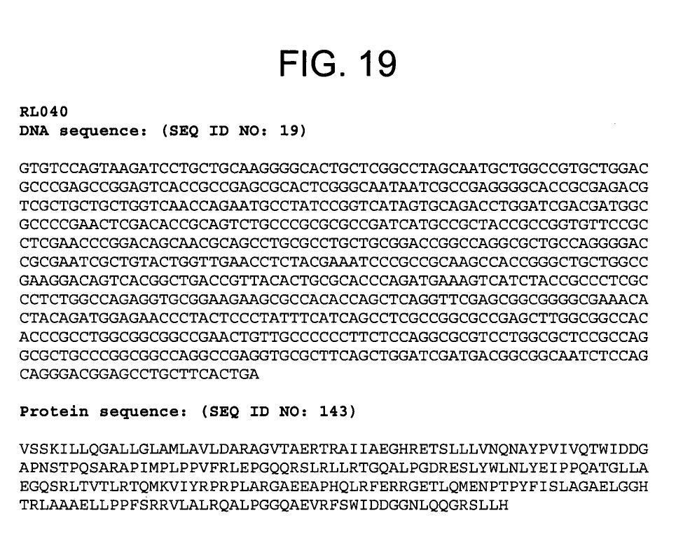

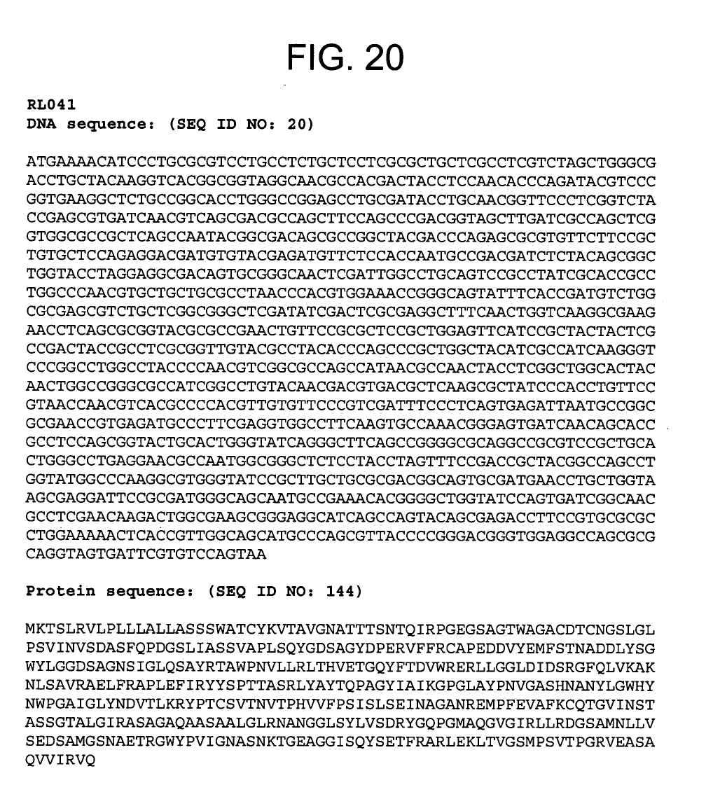

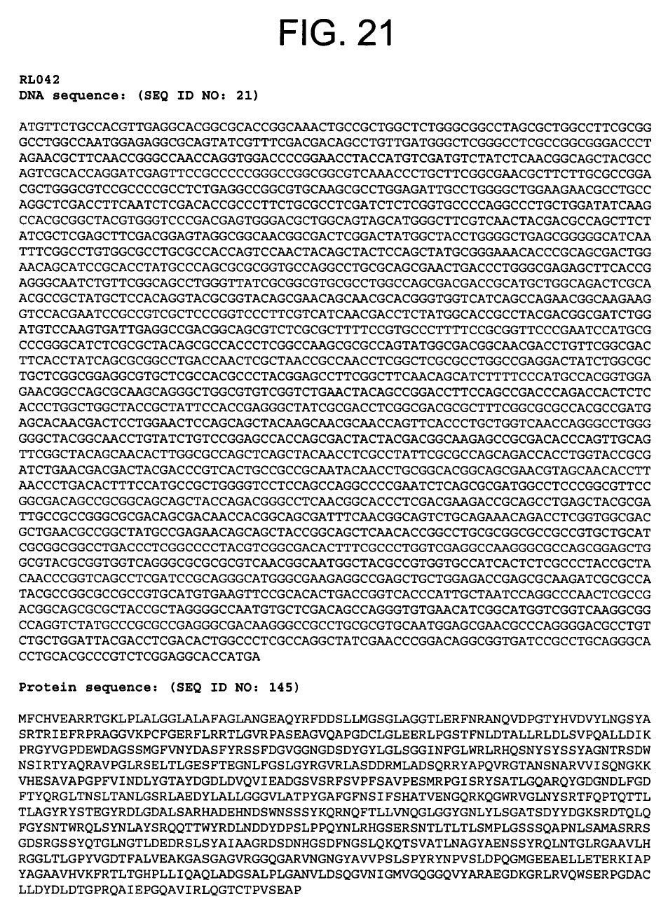

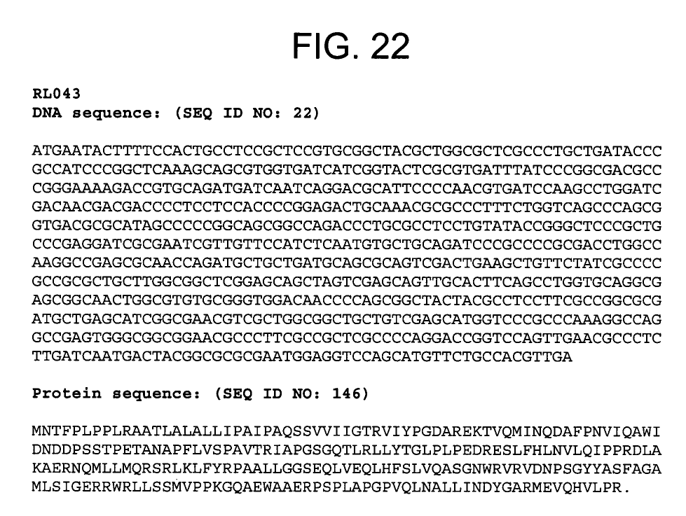

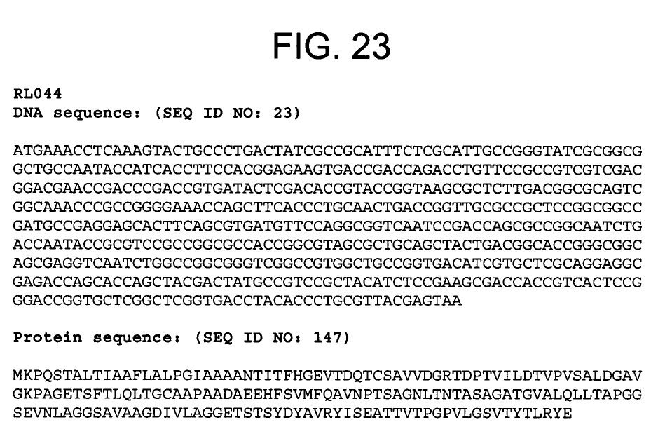

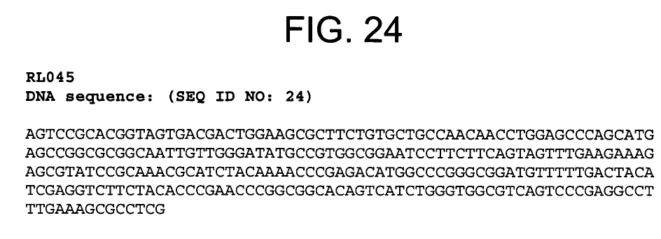

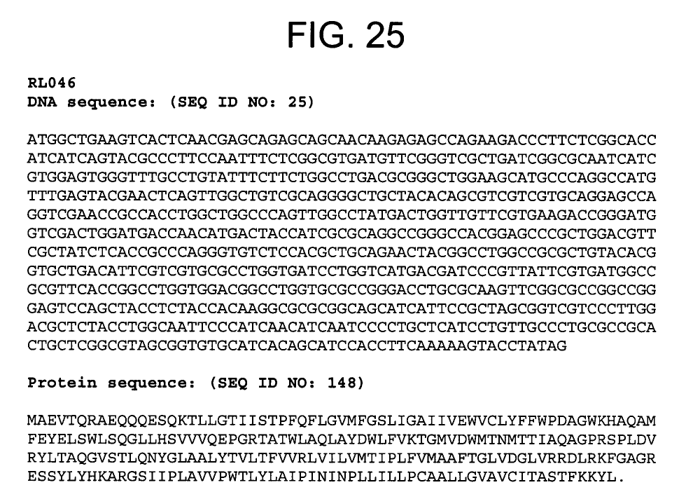

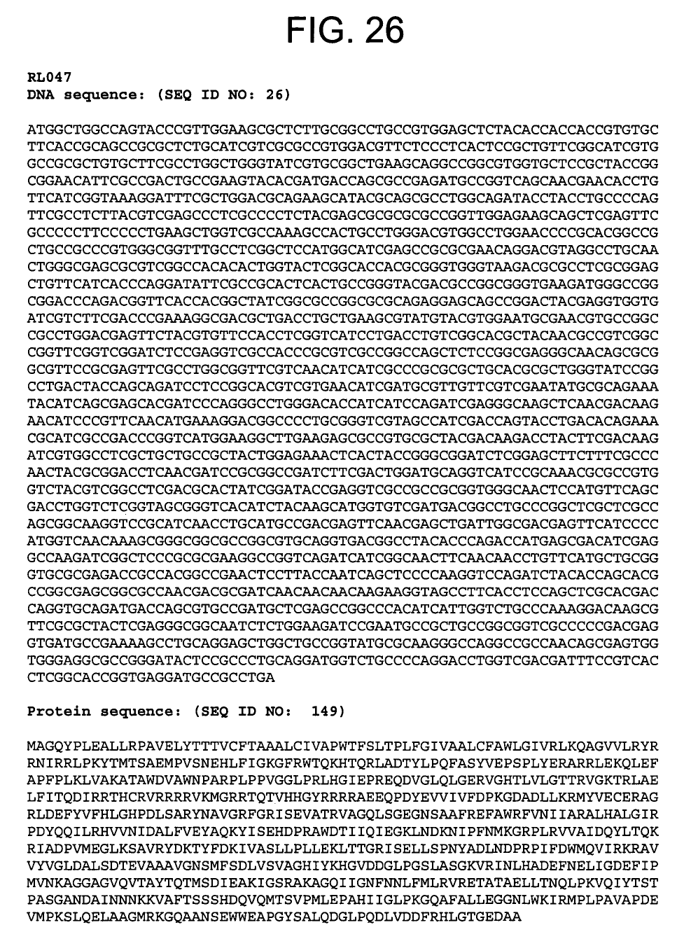

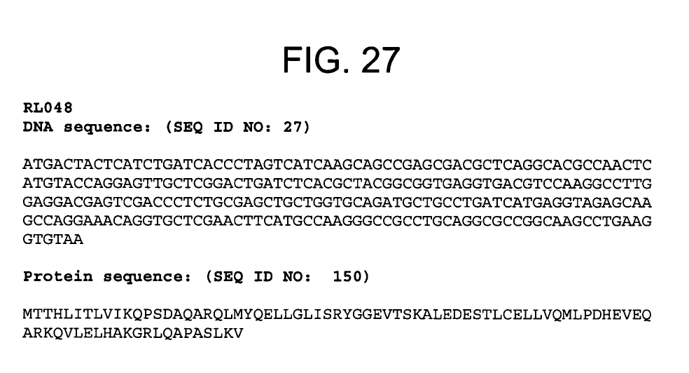

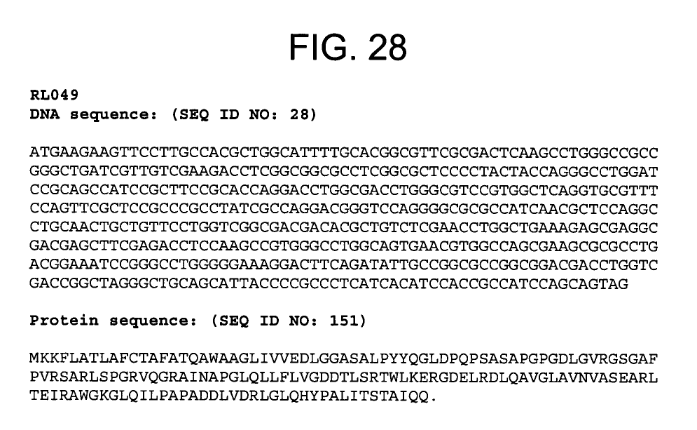

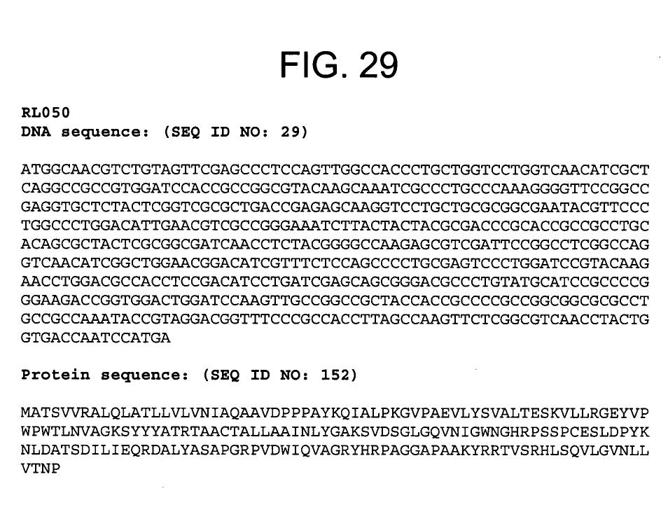

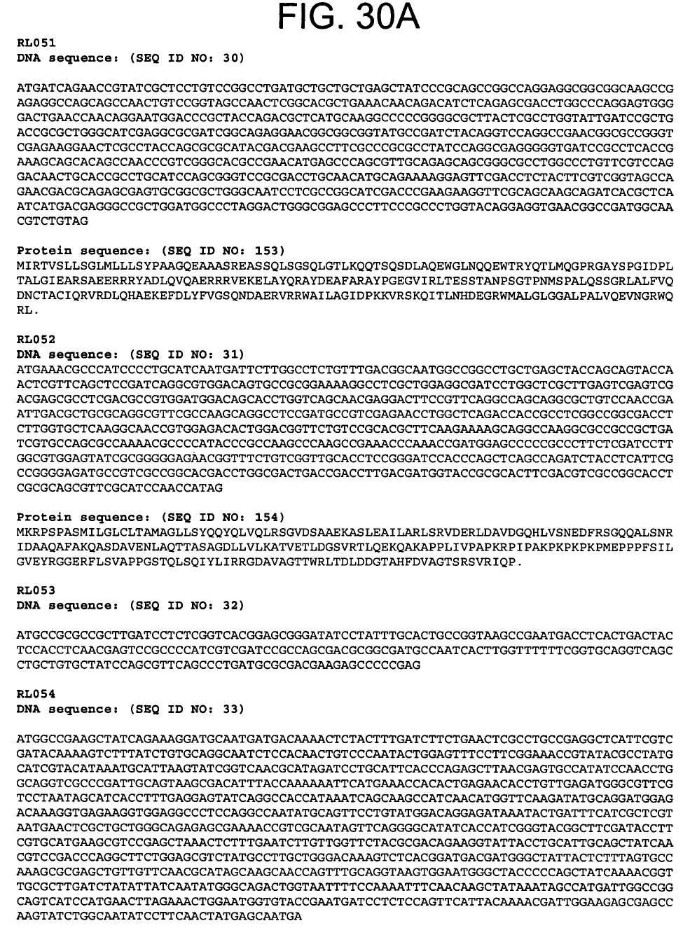

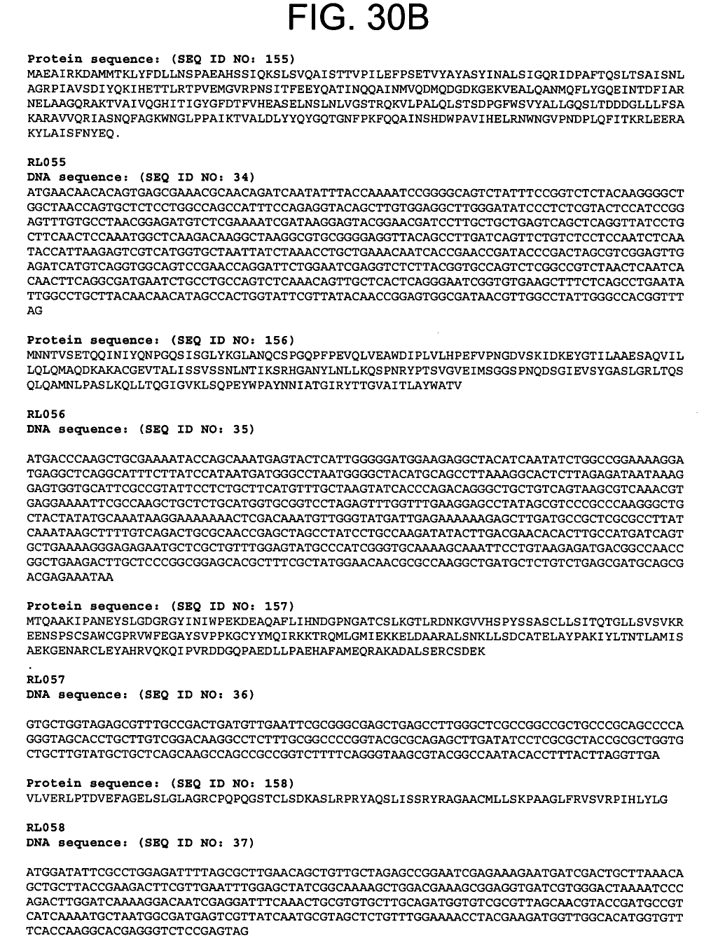

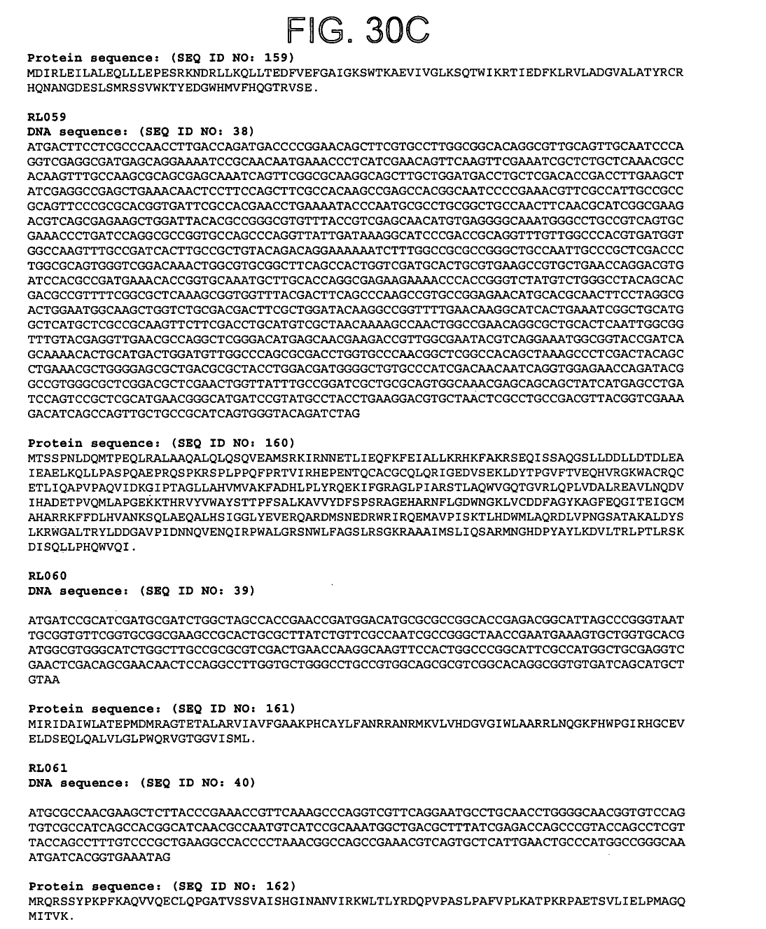

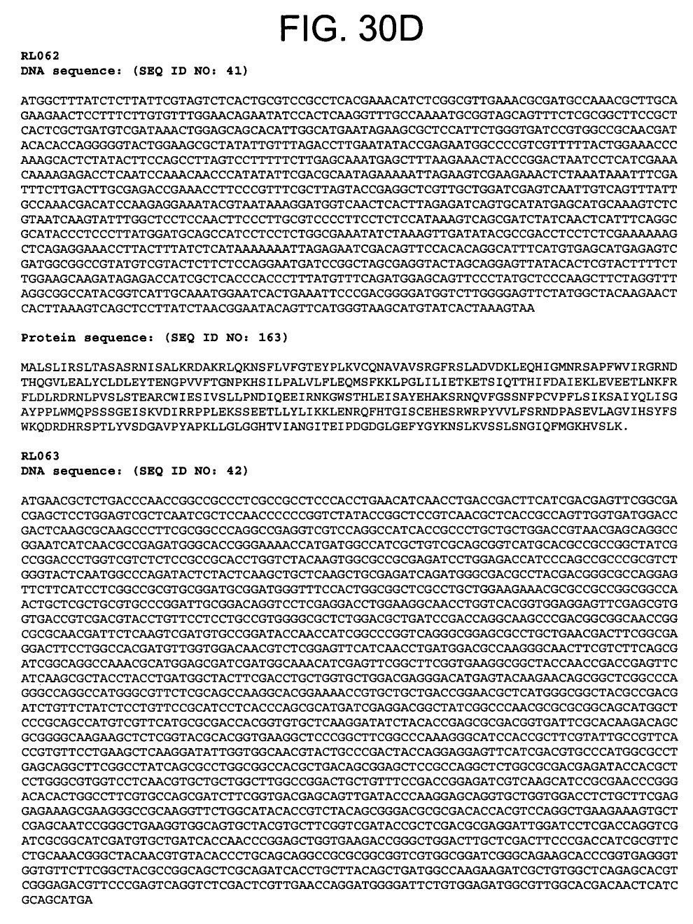

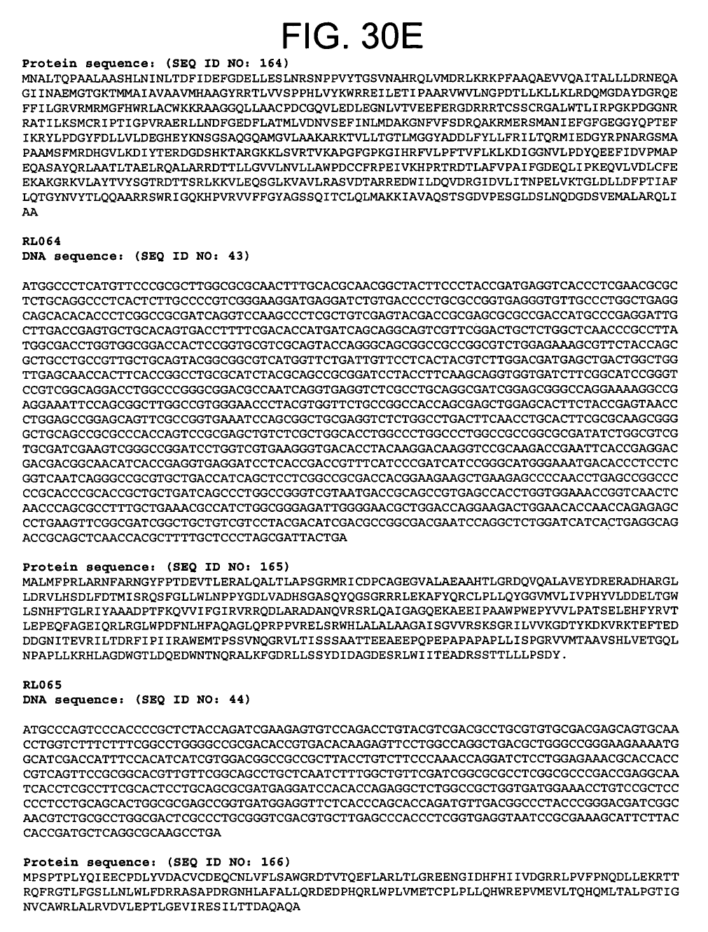

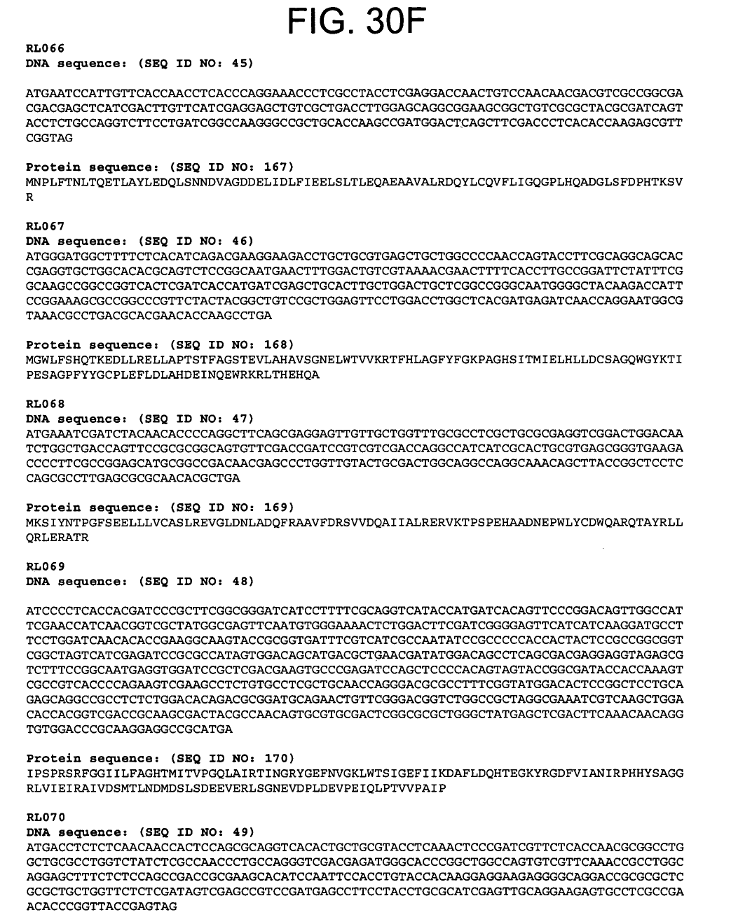

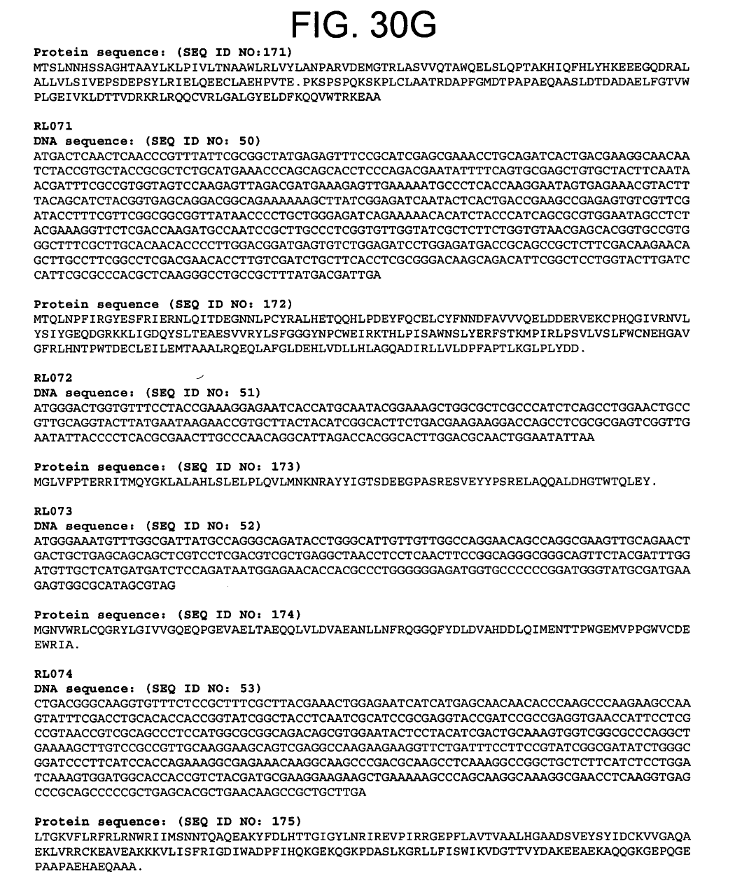

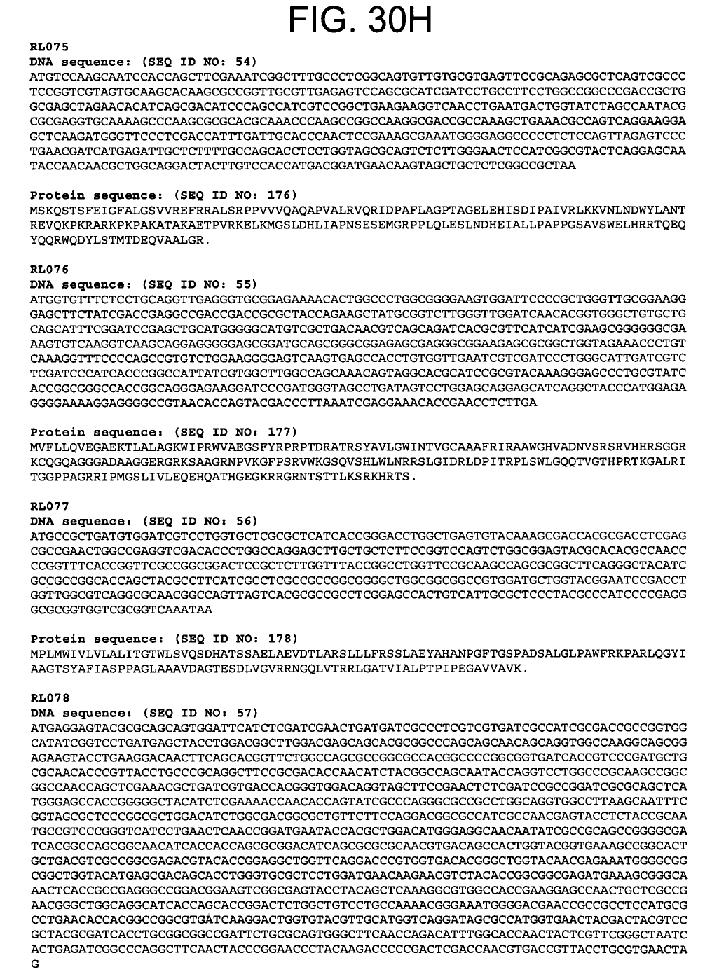

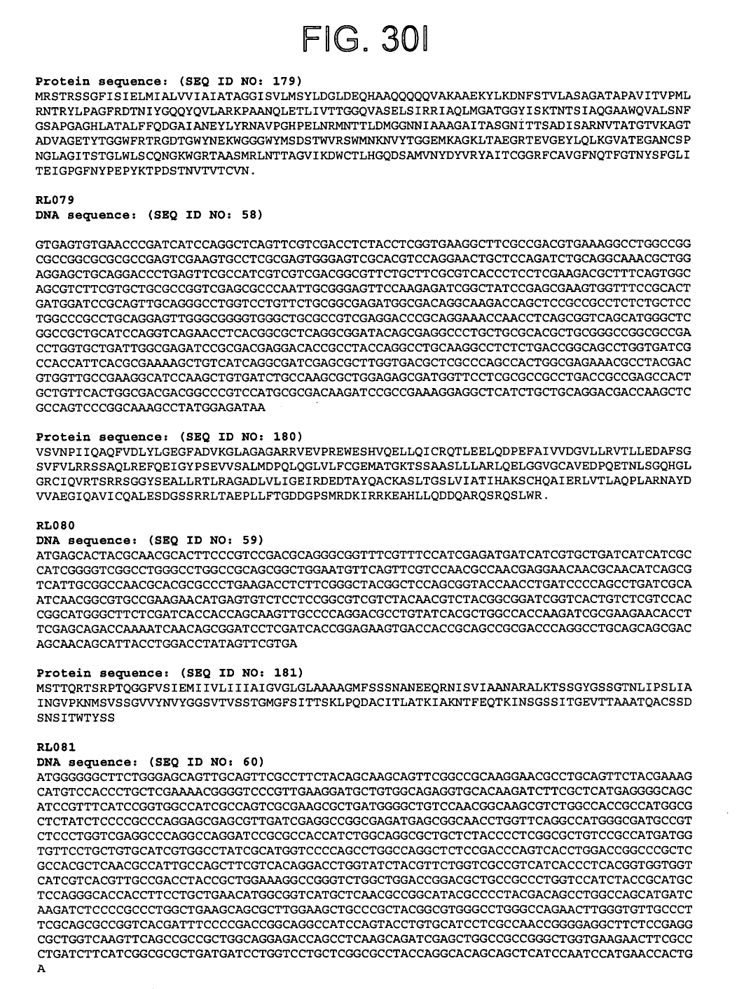

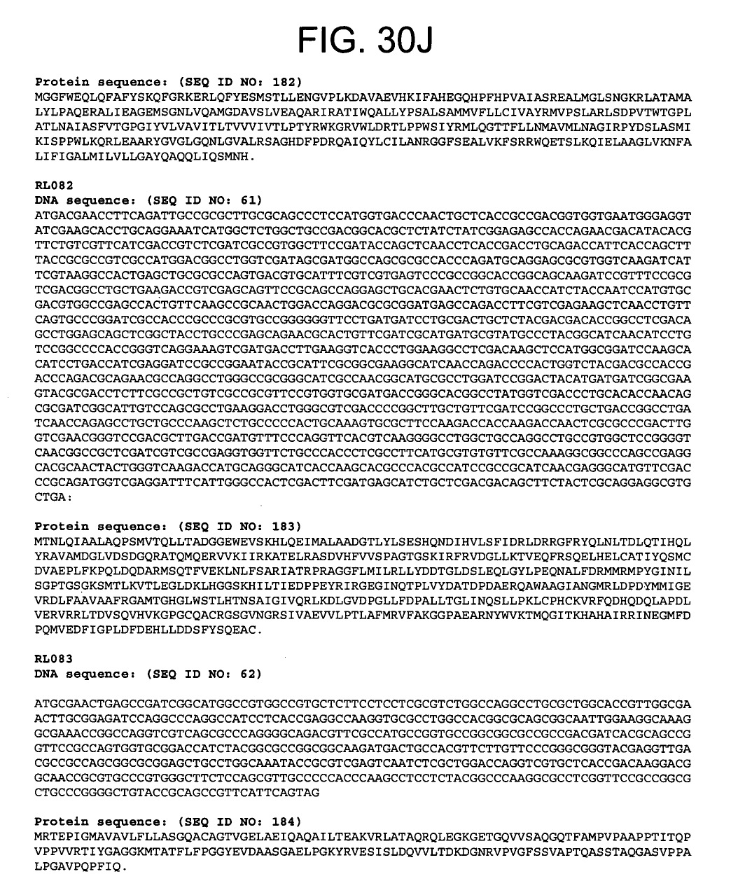

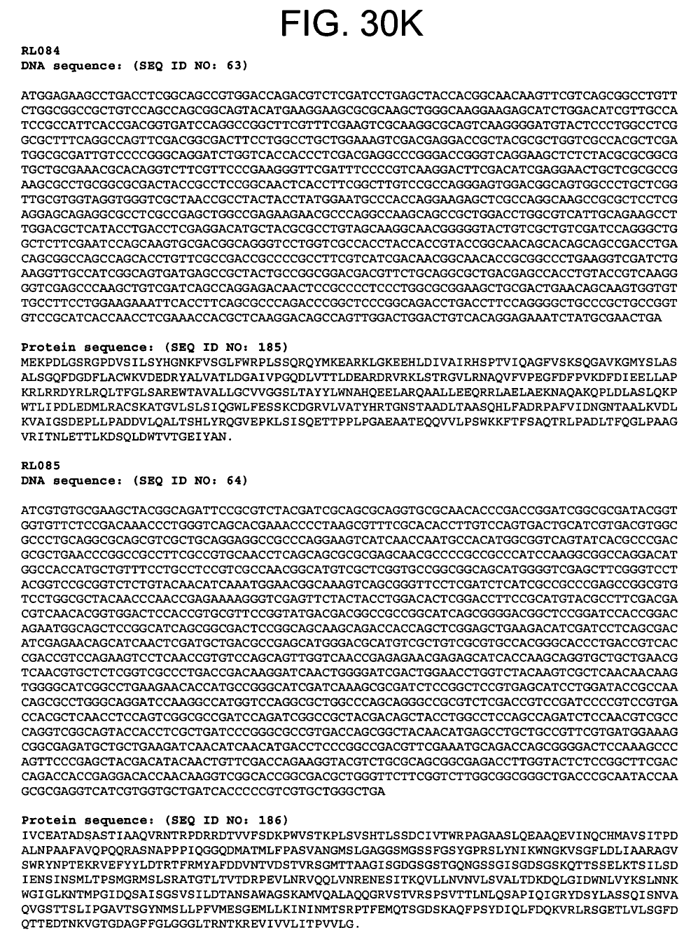

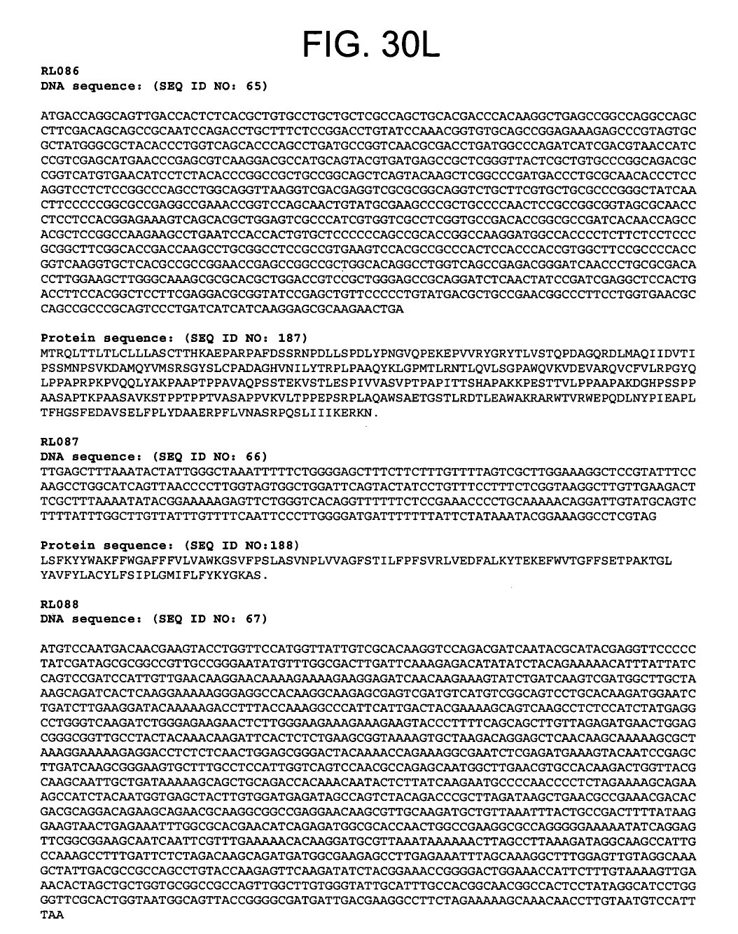

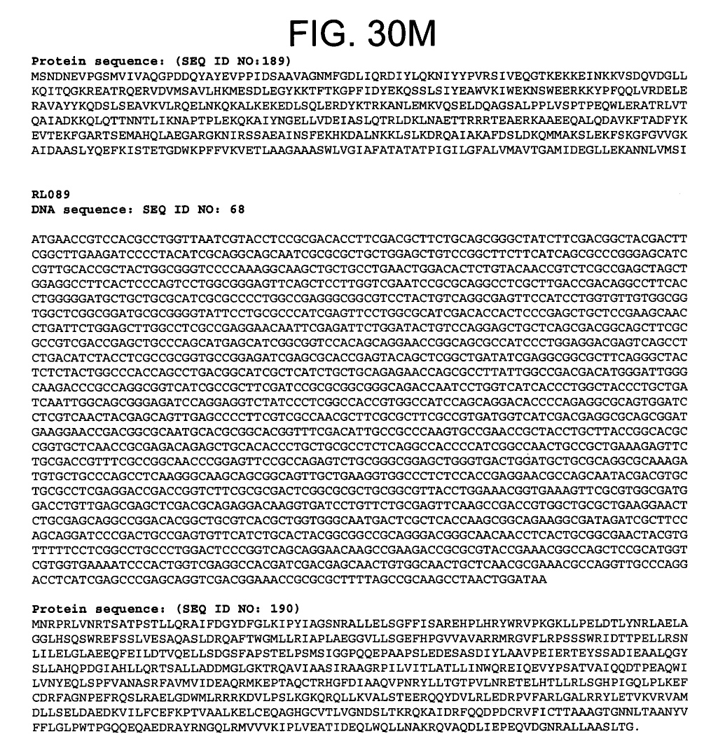

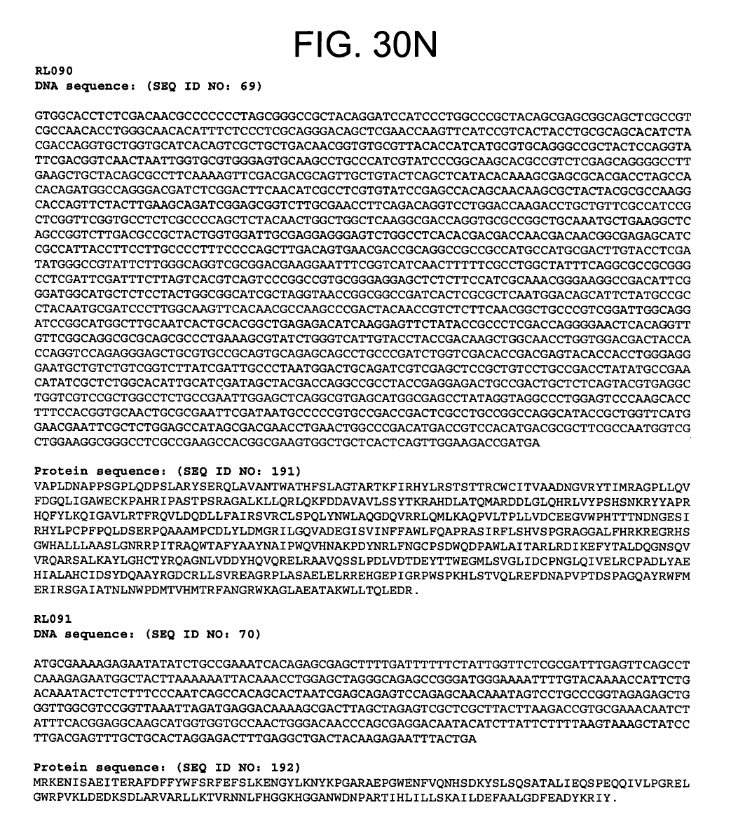

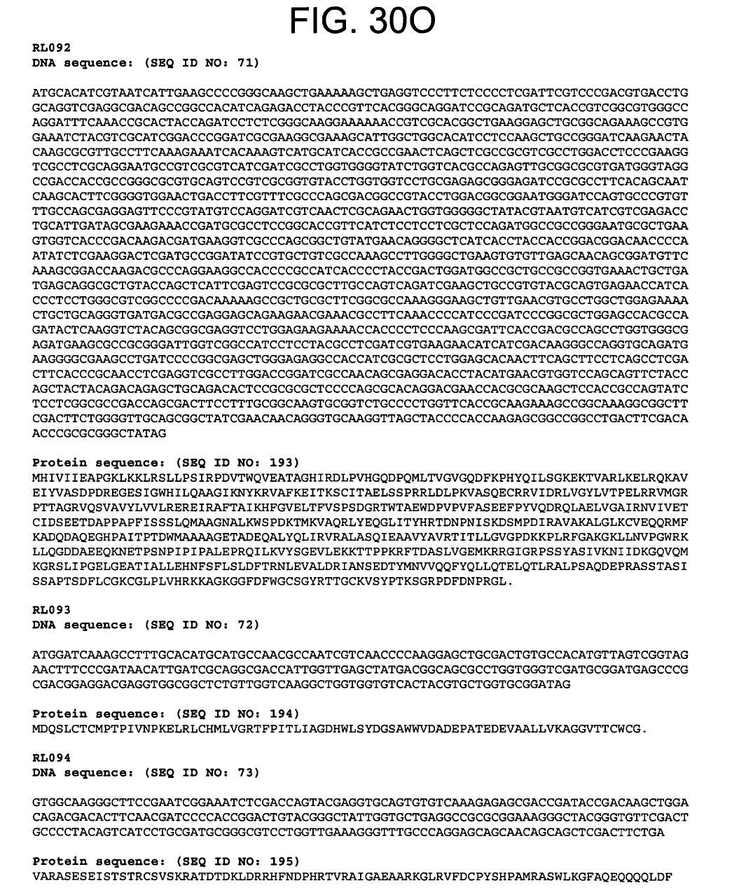

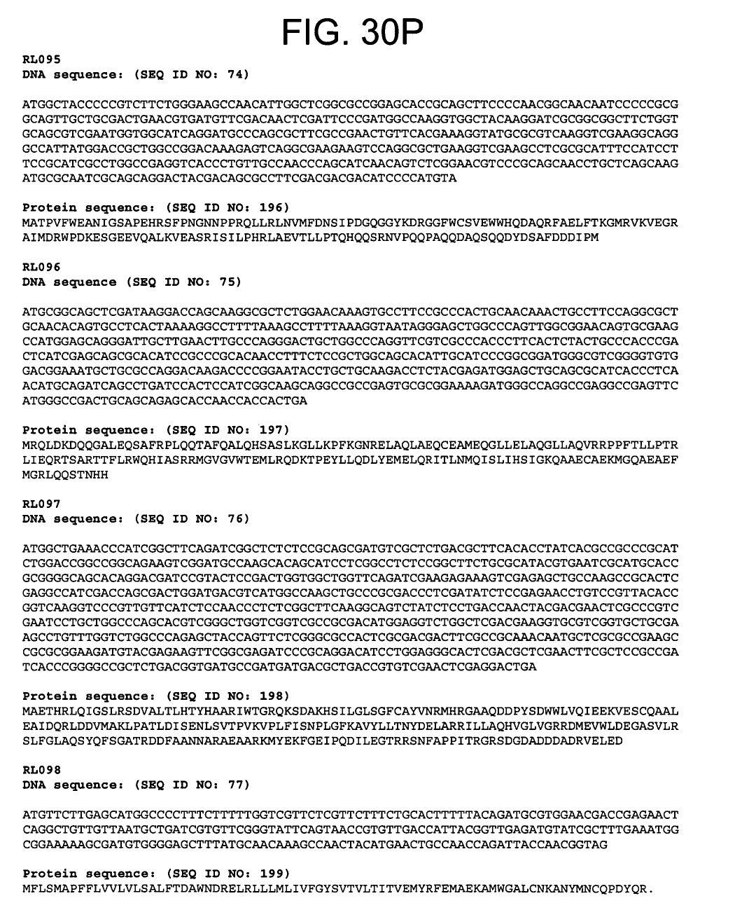

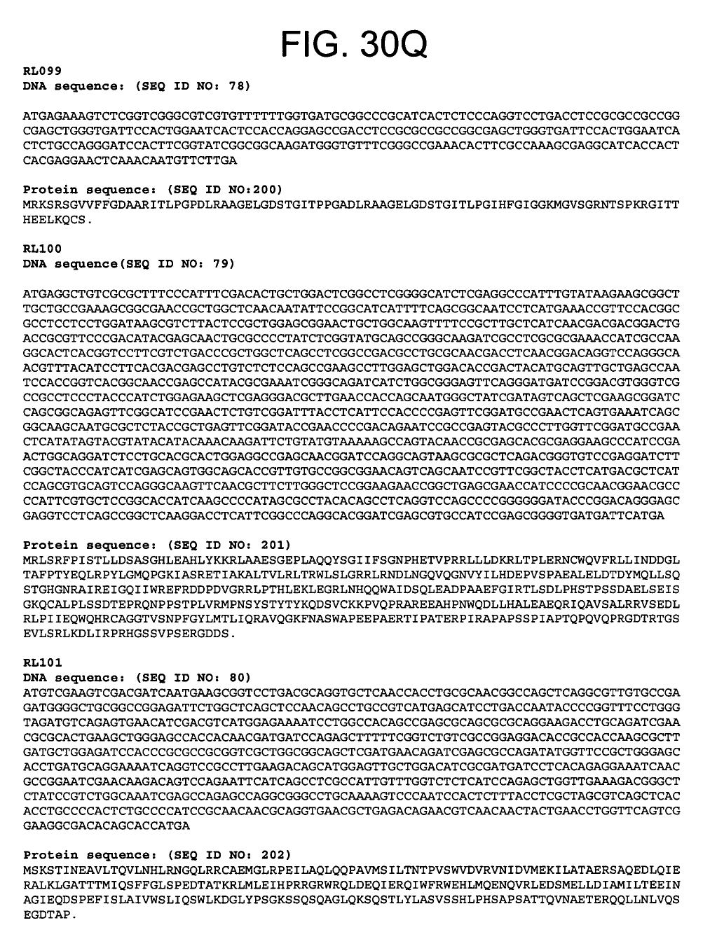

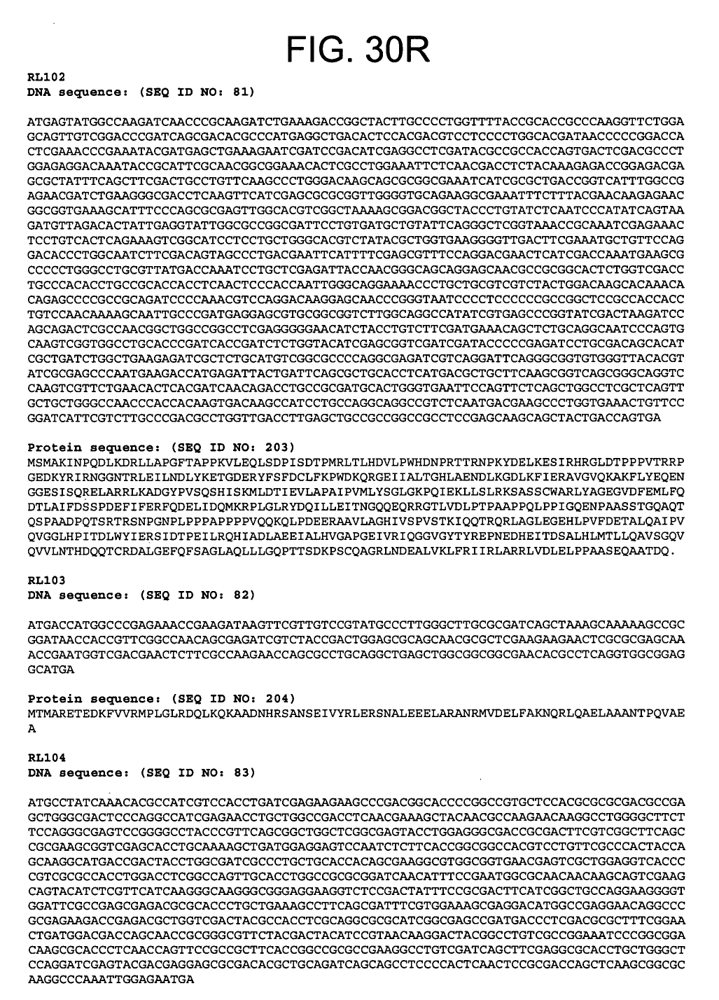

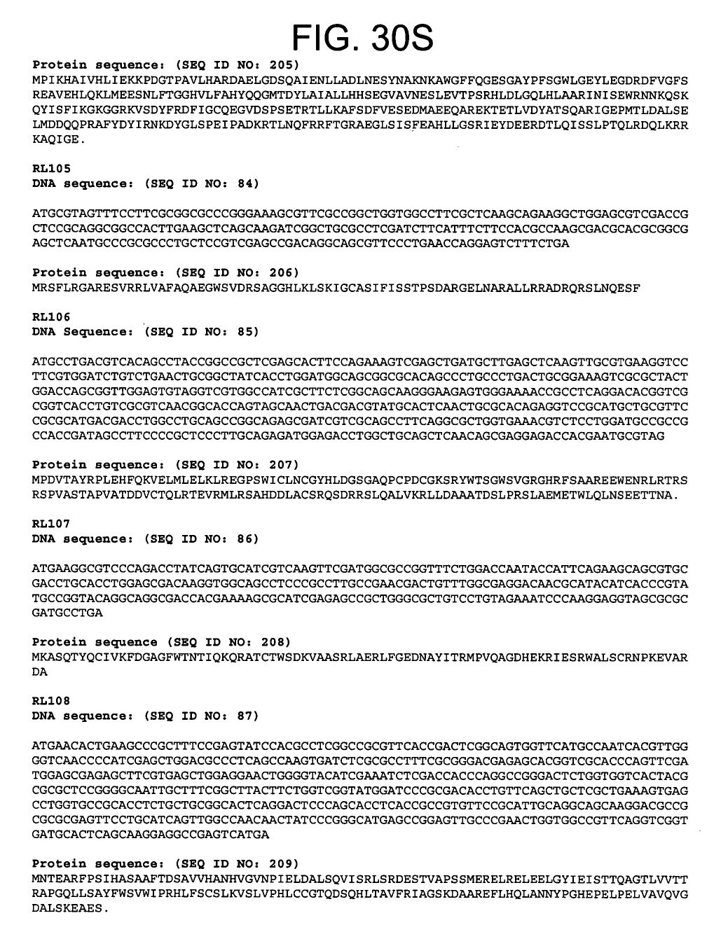

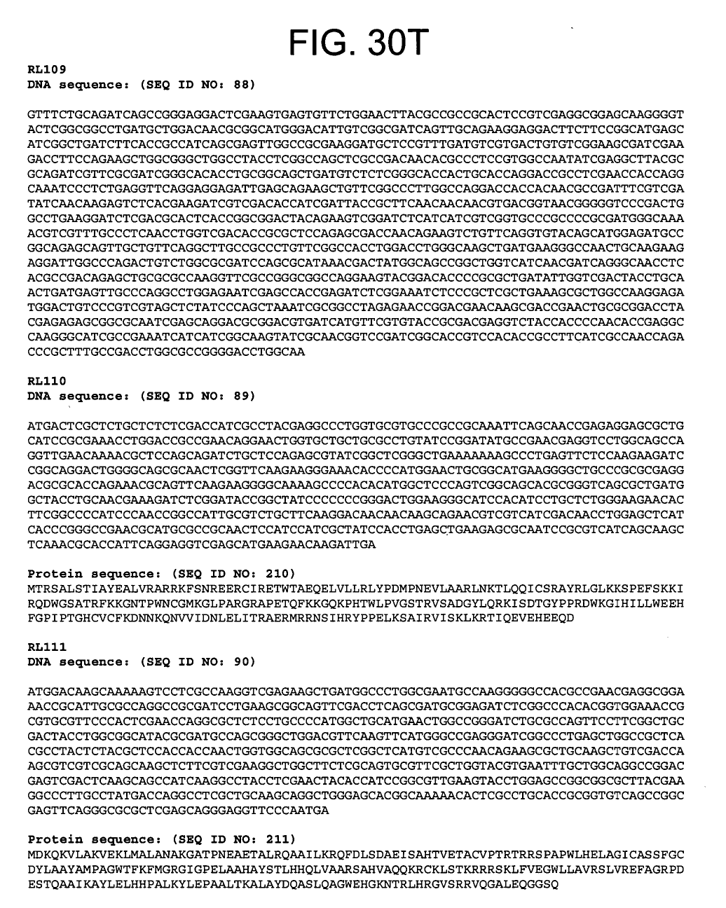

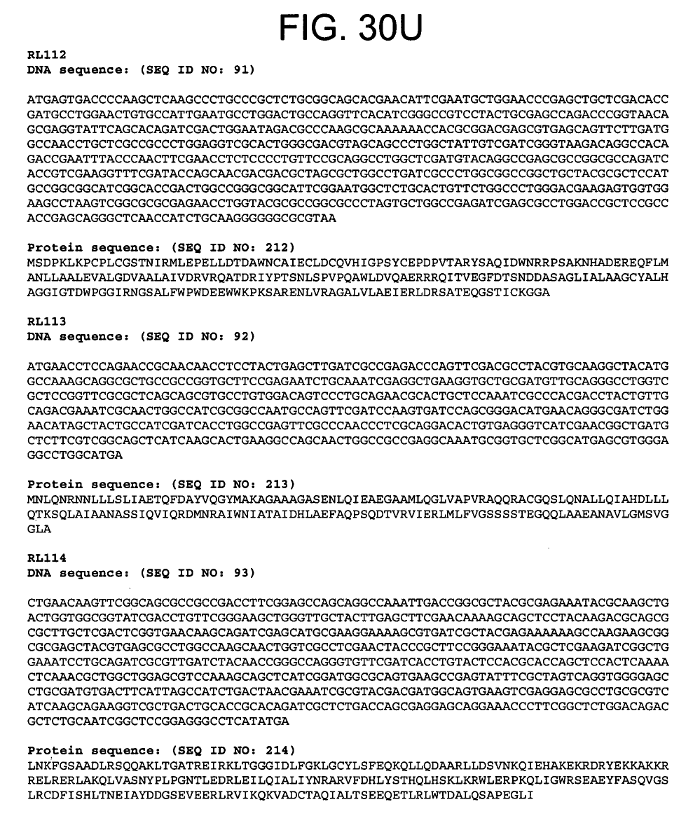

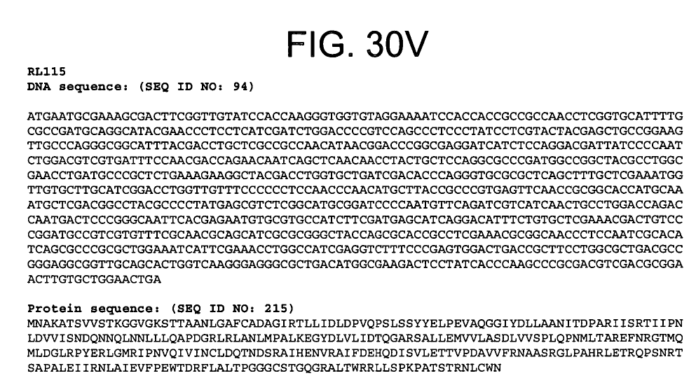

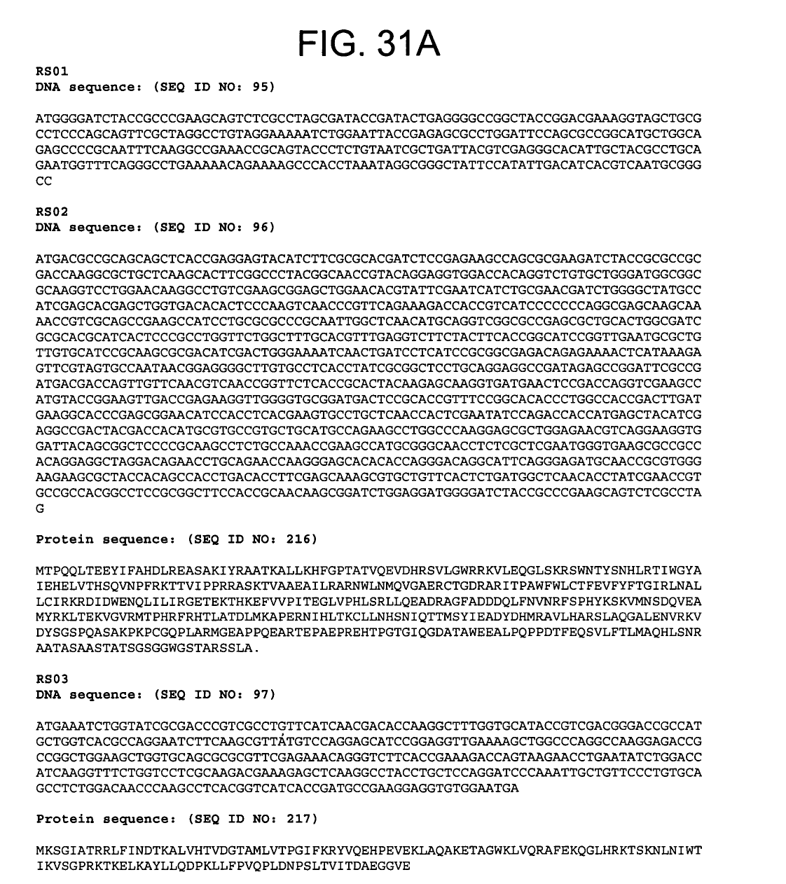

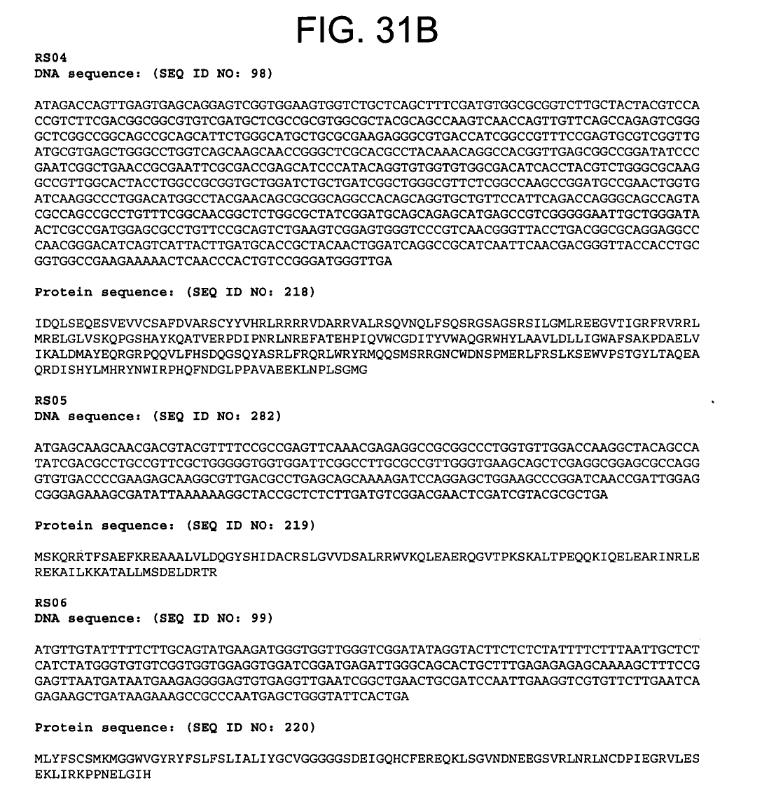

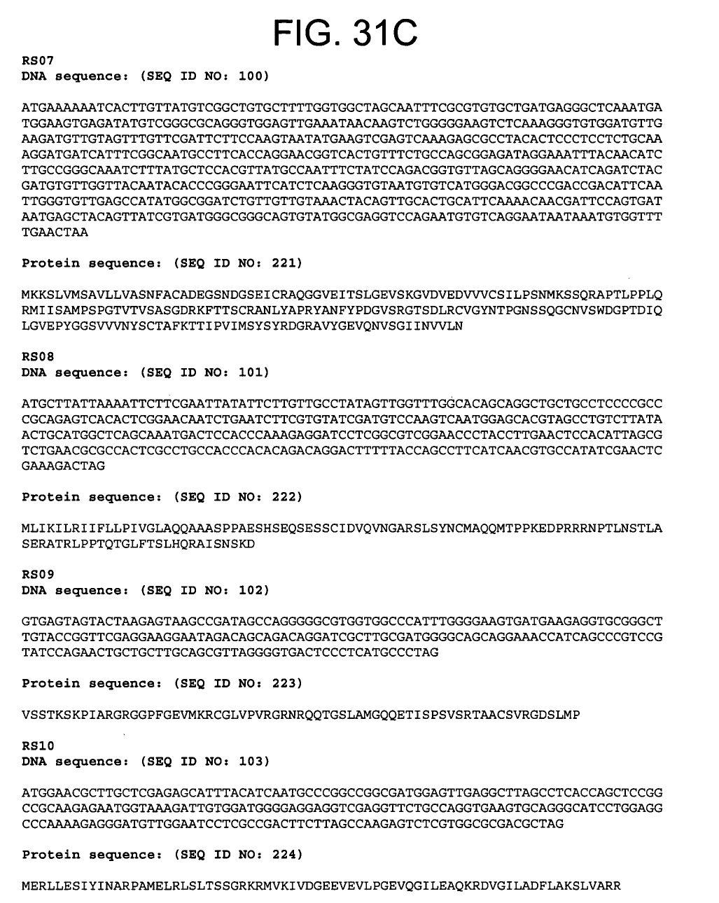

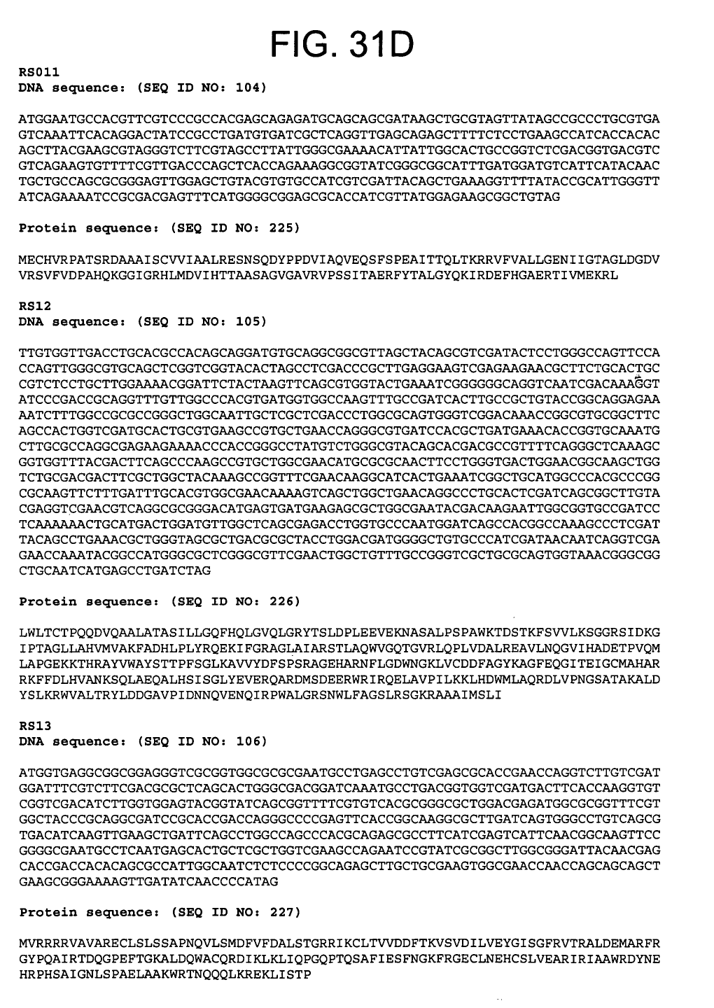

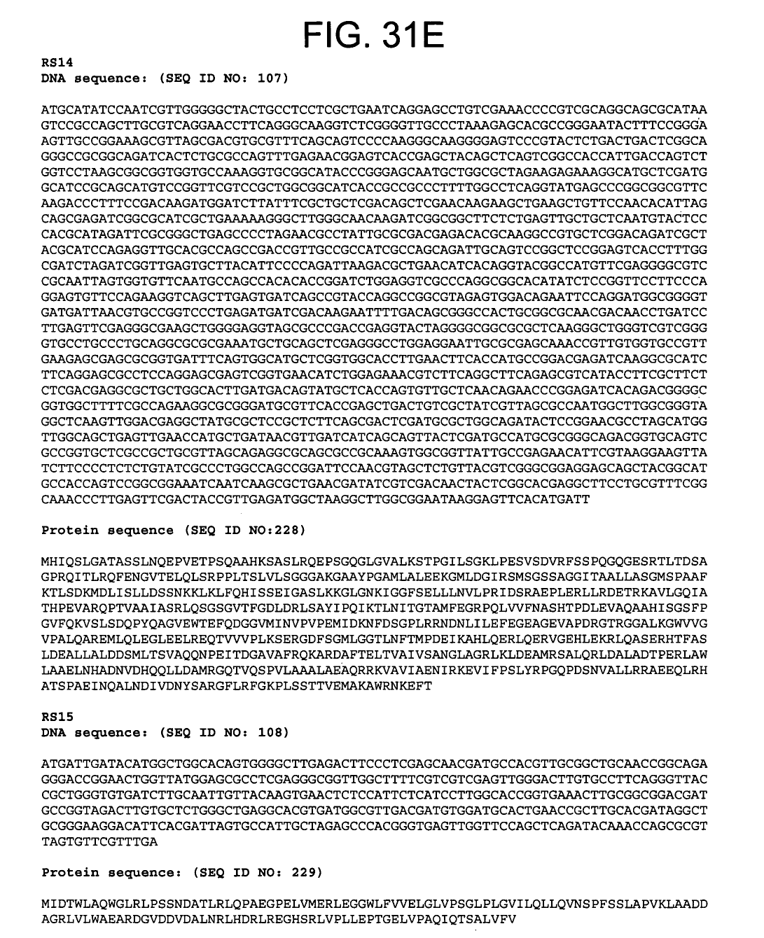

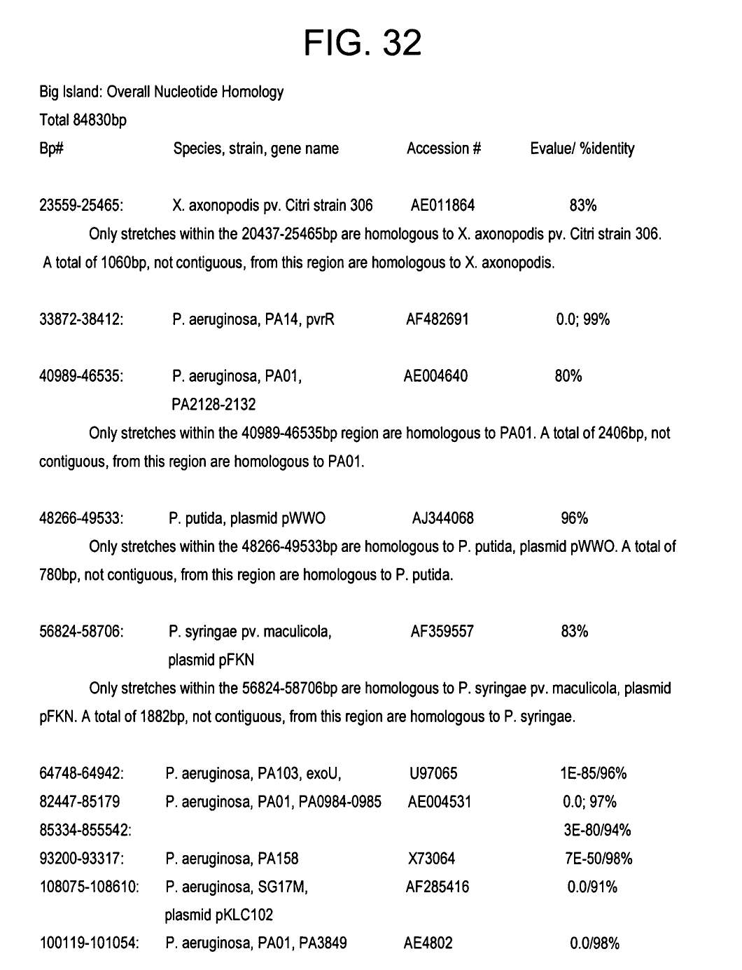

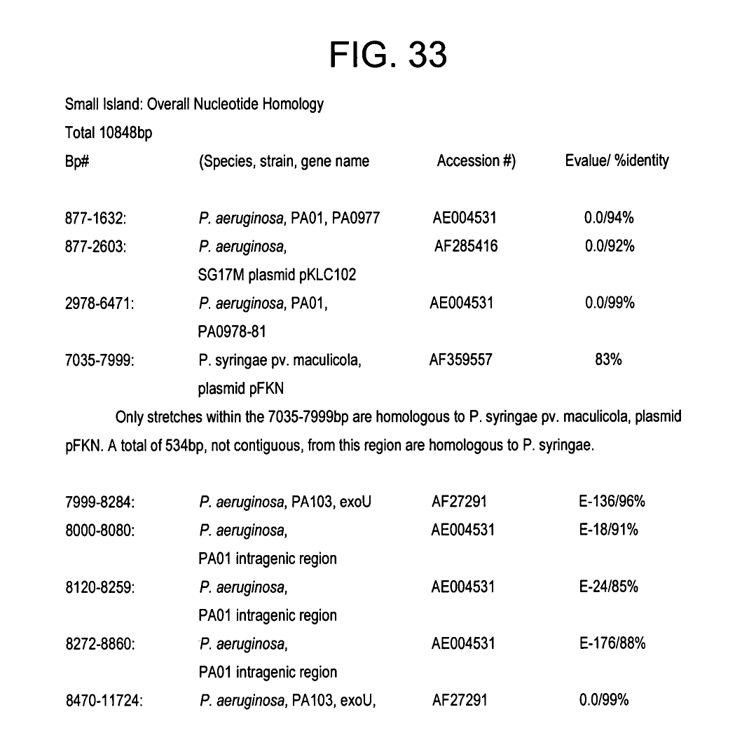

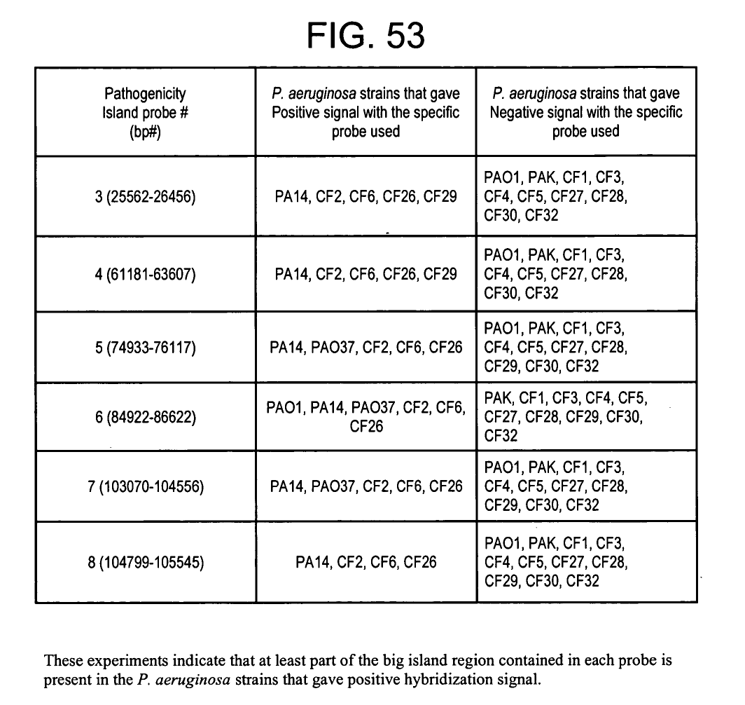

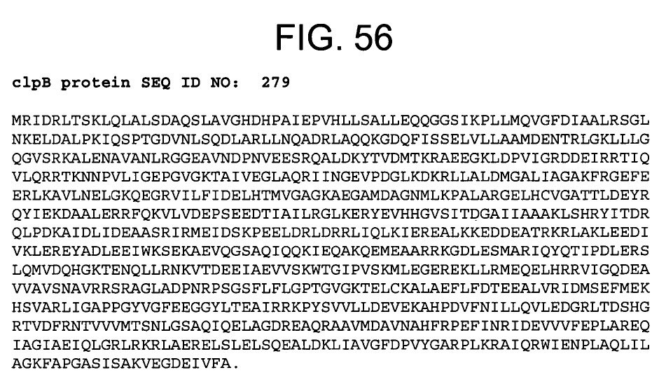

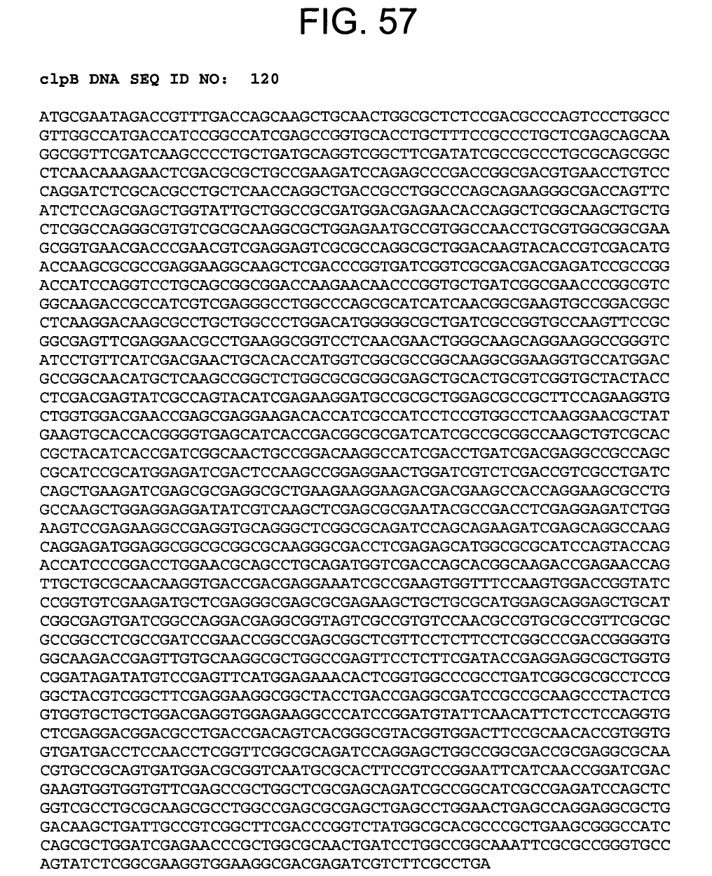

The present invention is based, in part, on the identification, sequencing, and characterization of two novel virulence islands of P. aeruginosa strain PA14, namely the P. aeruginosa pathogenicity island-1 (PAPI-1) (large pathogenicity island, GenBank Accession Number AY273869 and SEQ ID NO: 2) and PAPI-2 (small pathogenicity island, GenBank Accession Number AY273870 and SEQ ID NO: 1). While the PAPI-1 is element is absent from the reference bacterial strain PAO1, a portion of the PAPI-2 element is found in this isolate. Our studies show that both islands (sequences are shown in FIGS. 1 and 2 as SEQ ID NO: 2 and SEQ ID NO: 1, respectively) contain novel virulence-associated factors involved in biofilm formation, surface adhesion, host invasion, toxin production, pili assembly, and/or fimbrial biogenesis. Exemplary genes (SEQ ID NOs: 1-108, SEQ ID NOs: 119-120, and SEQ ID NOs: 281-282) and their translation products are described in FIGS. 3-31 and 54-57 (SEQ ID NOs: 127-229 and SEQ ID NOs: 278-280). FIGS. 32-34 and 58 describe many other characteristics and functions of the nucleic acids and proteins of the invention. FIG. 53 further demonstrates that these nucleic acids are found within a variety of pathogenic P. aeruginosa strains. Furthermore, the encoded proteins play an important role in the pathogenesis of P. aeruginosa. Interestingly, most of the predicted proteins encoded by the PAPI genes share no homology with any proteins of known function. By mutating several of these genes, we demonstrate their relevance both in plant and animal pathogenicity. Thus, based on our results, PAPI-1 and PAPI-2 virulence factors promote the broad host promiscuity of highly virulent P. aeruginosa strains, such as PA14, relative to less virulent strains such as PAO1. Furthermore, our results provide support for the implication of the modular structure of pathogenicity islands in the evolution, relatedness to other bacterial species, and the generation of pathogenic variants.

In addition to novel genes, these islands further contain a number of genes encoding for transposases, helicases, inverted repeats, and tRNA sequences at the borders of these islands, confirming that these genomic regions correspond to genomic islands. PAPI-1, the big pathogenicity island, contains 115 ORFs, 92 of which are described herein and the remainder are described in “Virulence-Associated Nucleic Acid Sequences and Uses Thereof,” U.S. Pat. No. 6,355,411, issued Mar. 12, 2002. PAPI-2, the small PA14 pathogenicity island described herein contains 15 ORFs. The only homology between these islands occurs with the PAPI-1 ORFs RL003 and RL059, which are homologous to PAPI-2 ORFs RS03 and RS12, respectively.

Identification of Two Genomic Islands in PA14 and Absent from PAO1

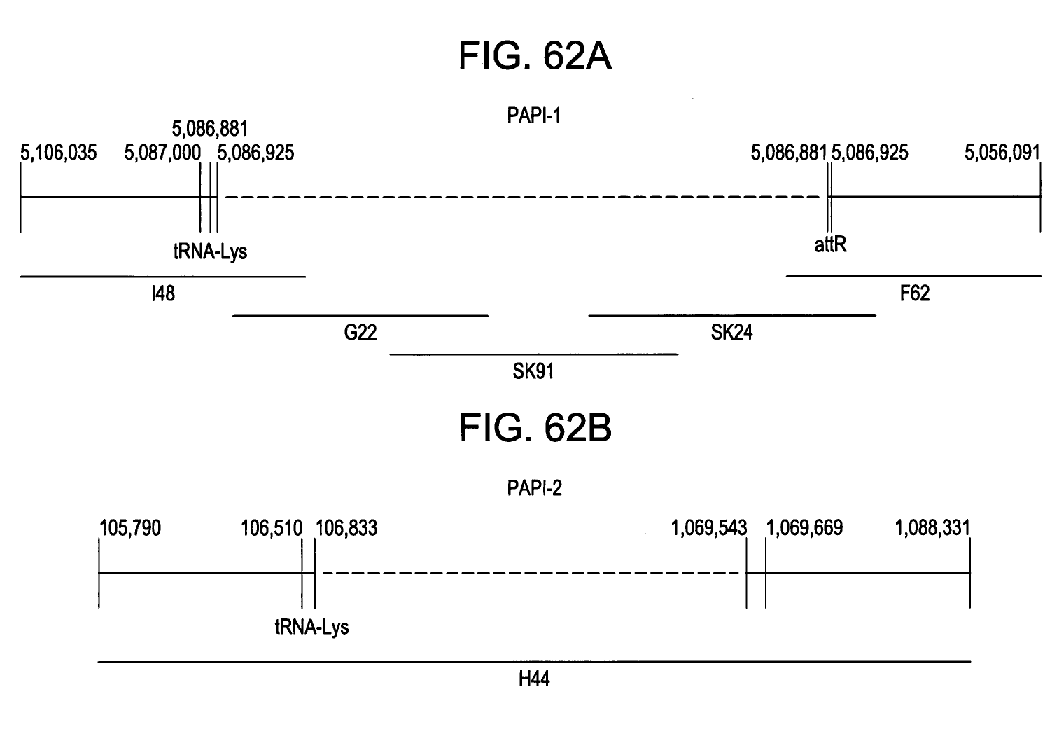

The PA14 isogenic mutant 33A9, which carries an RL003 gene mutation, exhibits reduced plant and mouse pathogenicity (Rahme et al., Proc Natl Acad Sci USA (1997) 94:13245-50). The fact that RL003 is absent from PAO1 is highly suggestive that it might occur within a P. aeruginosa pathogenicity island. Thus, we screened a PA14 cosmid library with a 300 bp RL003 probe. Initial results with the cosmids pA113, pB104, p148, pH44, and pG68 (as shown in FIGS. 62A and 62B) showed that only pA113, pB104 and pI48 overlap. Although both borders of pH44 and pG68 contain PAO1 sequences, only the left borders of pA113, pB104 and pI48 carry PAO1 DNA, indicating that RL003 occurs in at least two sites in the PA14 genome, one of which includes a large genomic block.

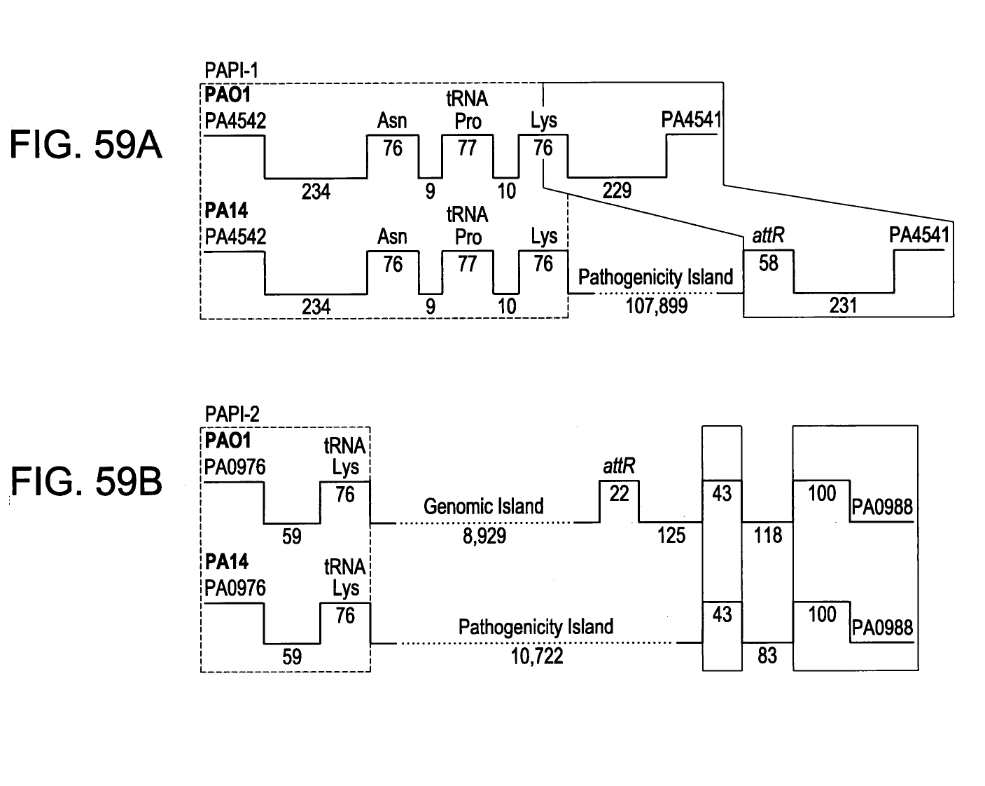

To further define this block, we carried out a progressive cosmid walk, starting with a p148 probe that contains the PAO1/PA14 left junction, and identified a cosmid carrying the right PA14/PAO1 junction (FIG. 62A). A set of five cosmid clones, p148, pG22, pSK91, pSK24 and pF62, were assembled to define a contiguous 150 kb region, designated PAPI-1, found in PA14 and absent from PAO1 (FIGS. 59A and 62A). Similarly, using probes that correspond to the right and left borders of pH44, we confirmed that this cosmid does not overlap PAPI-1, demonstrating that a second copy of RL003 occurs on a smaller PA14 genomic block, designated PAPI-2 (FIGS. 59B and 62B).

PAPI-1, the Large Pathogenicity Island

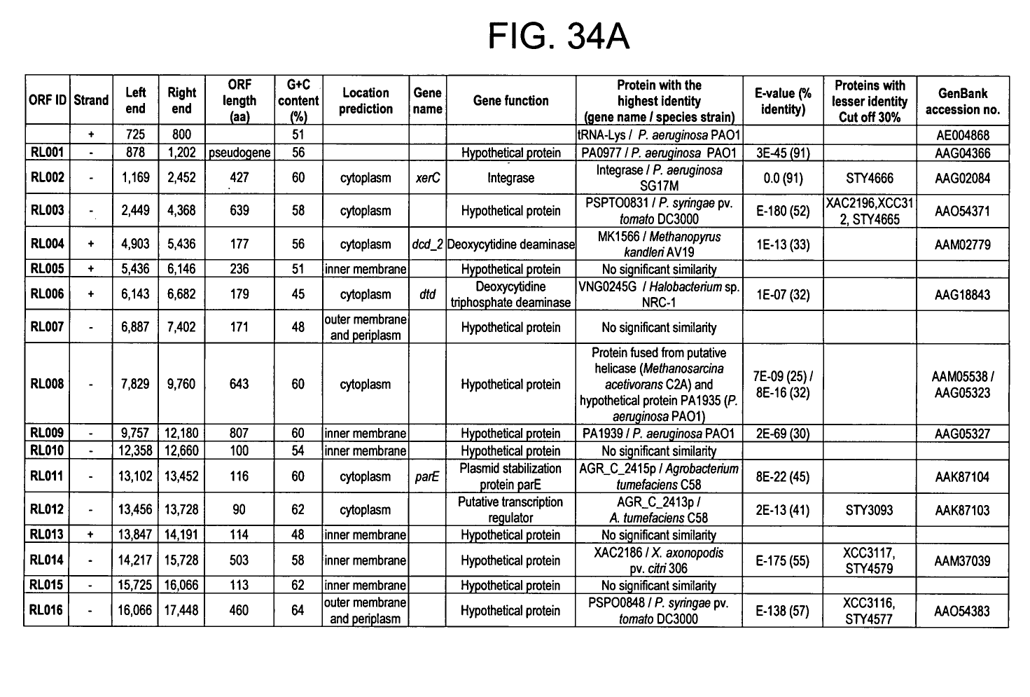

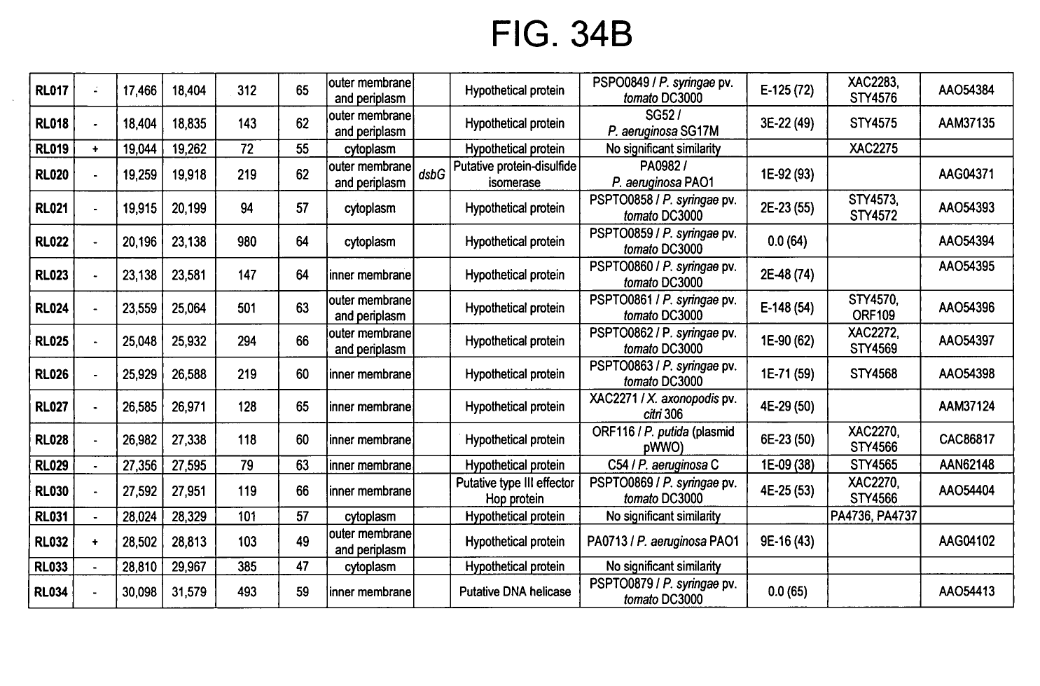

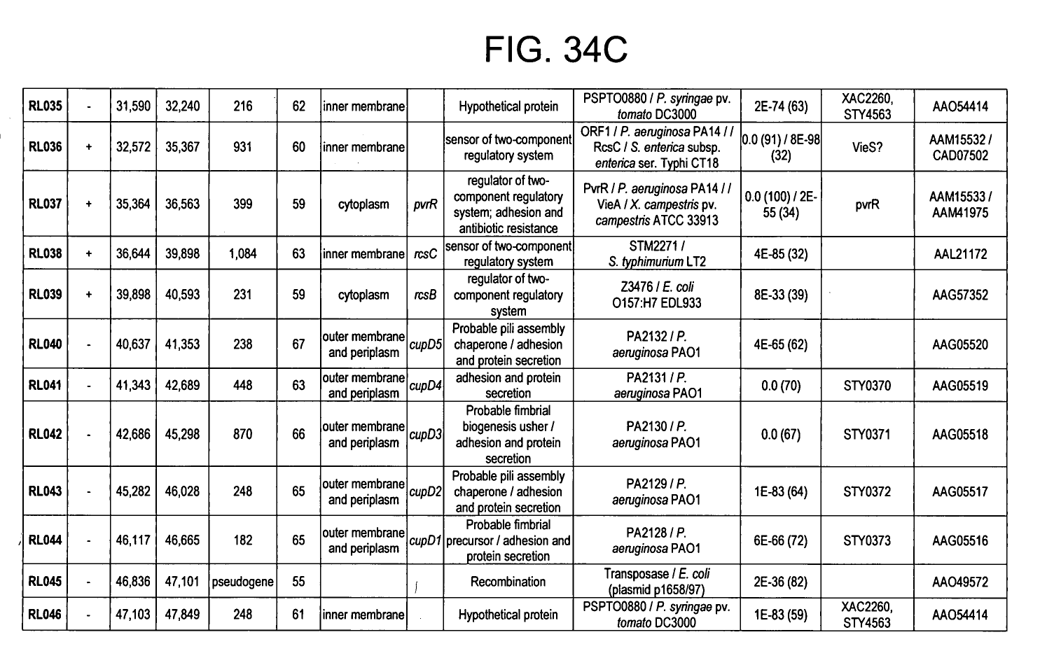

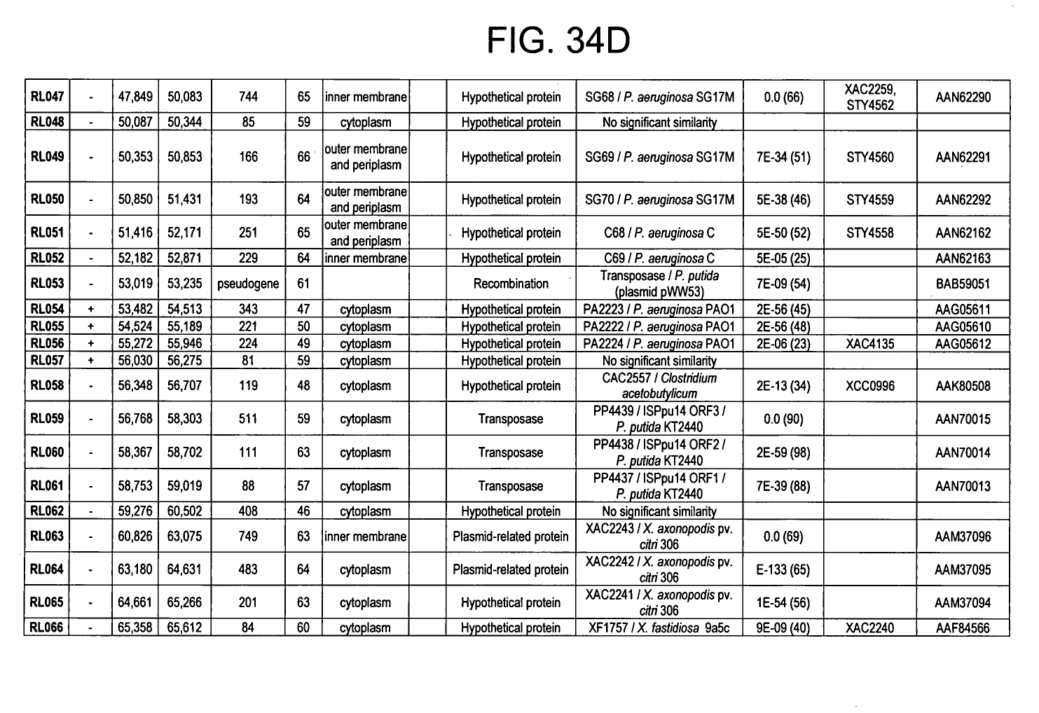

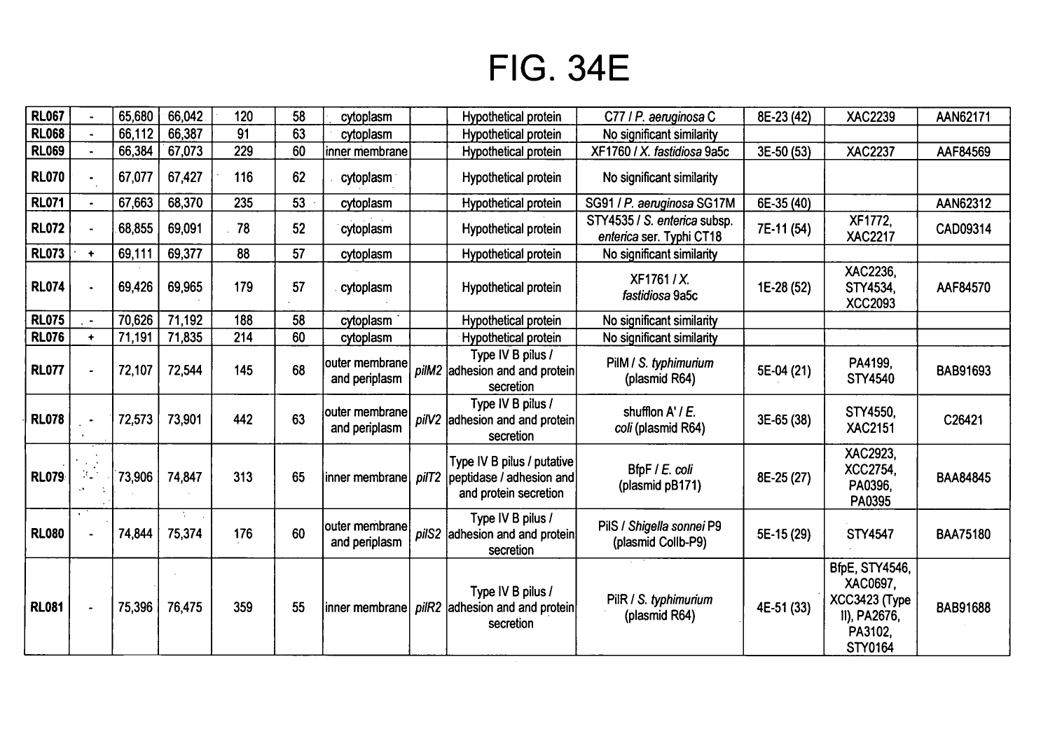

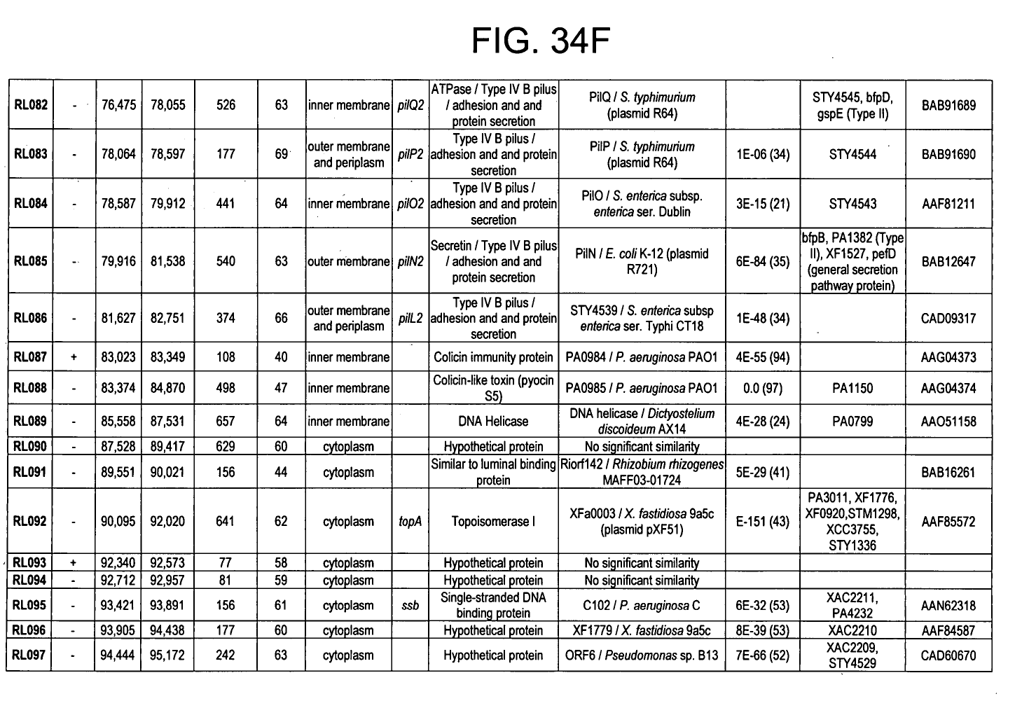

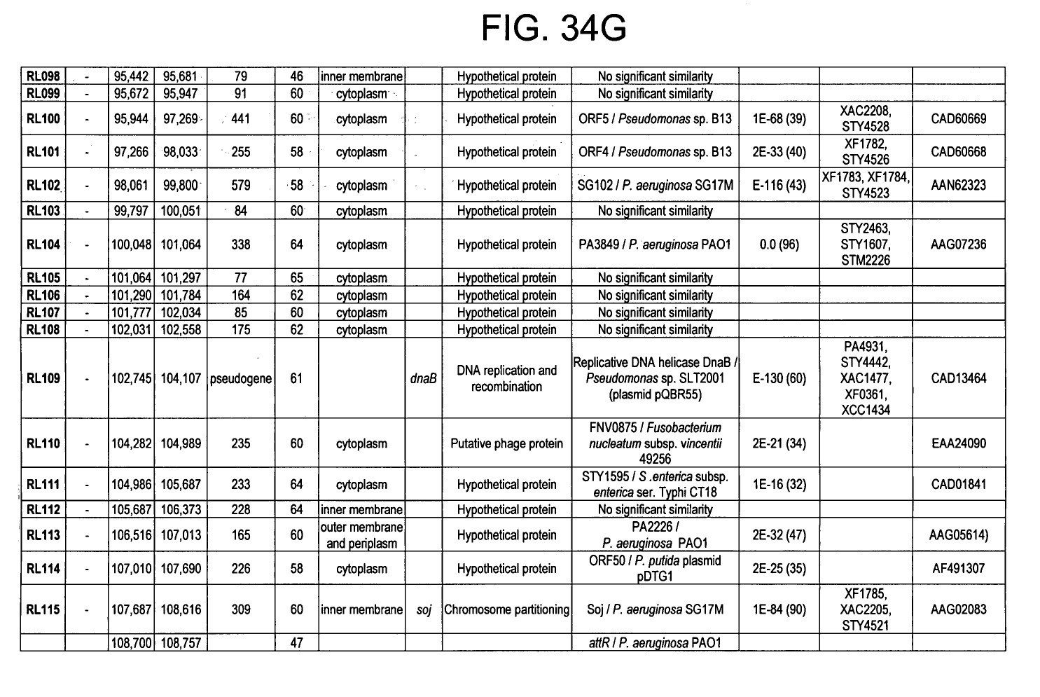

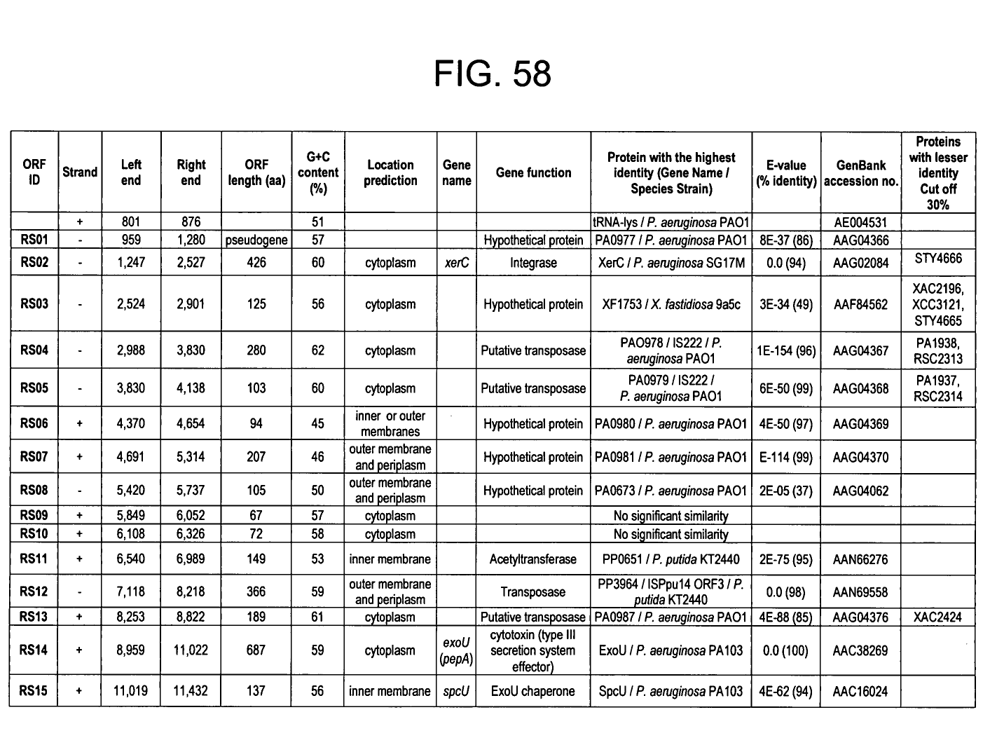

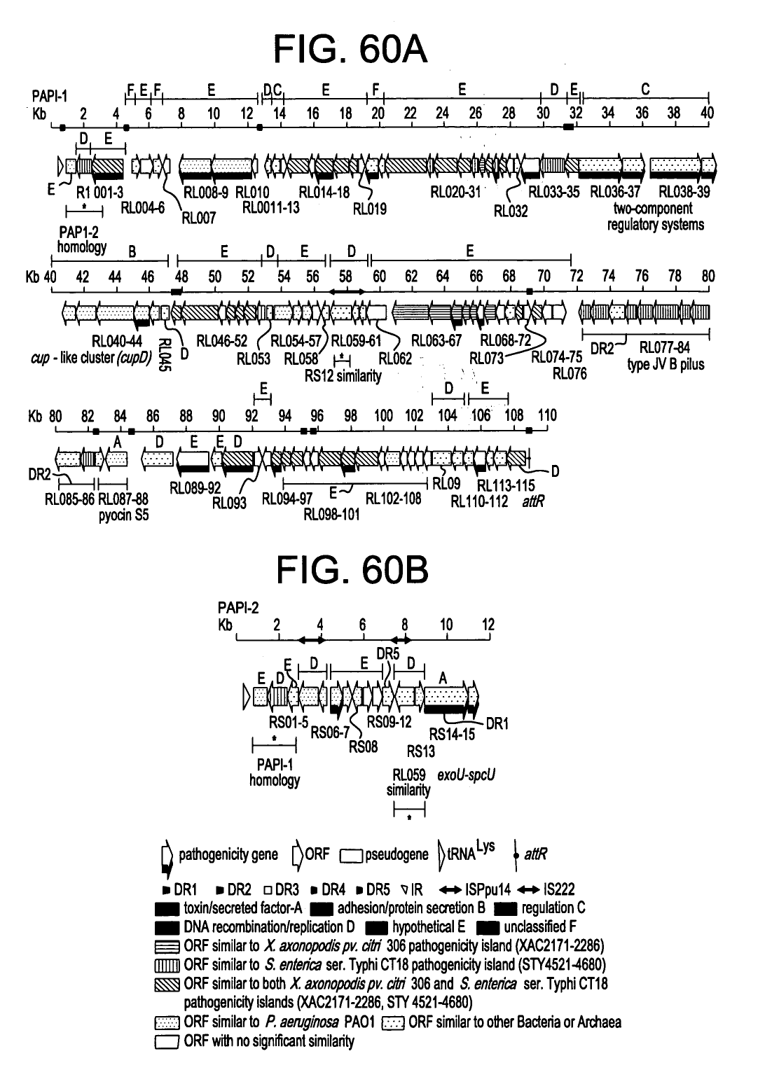

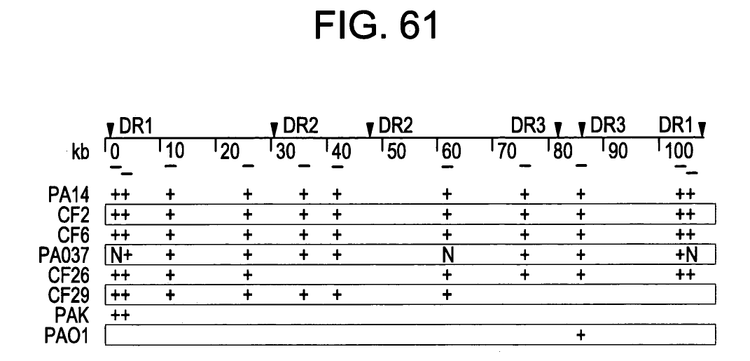

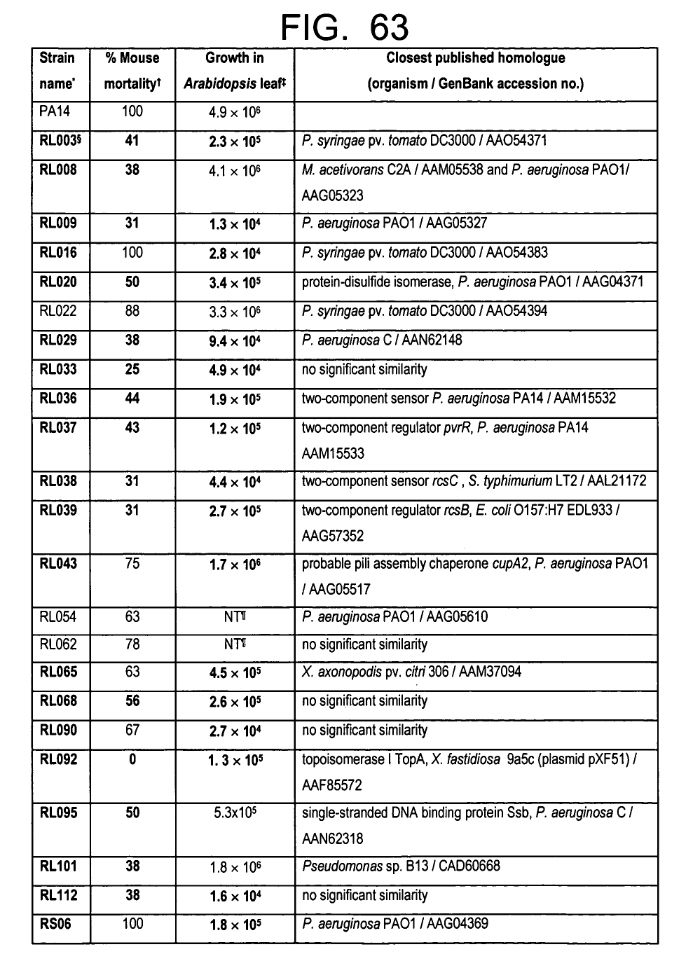

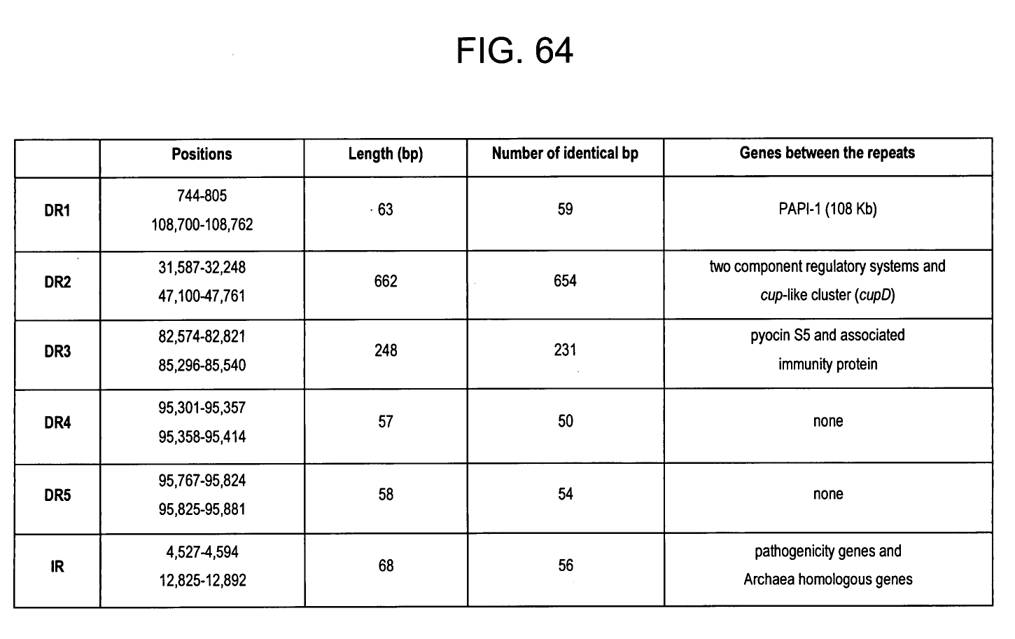

Comparison of the nucleotide sequence of the region defined by the five PAPI-1 cosmids (annotated in FIGS. 34 and 60A) with the PAO1 genome (Stover et al, Nature (2000) 406:959-64), shows that the 20 kb (GenBank Accession Number AY273871) left end of pI48 is collinear with the PAO1 genome, while the internal 107,899 bp are unique to PA14 (FIGS. 59A and 60A). This 108 kb region has all the features of a genomic island: it occupies a block absent from several P. aeruginosa strains (FIG. 61); its G+C content (59.7%) is different than that of the core genome (66.6%); it is associated with tRNA genes, as a tRNA^{ASn}, tRNA^{Pro} and tRNA^{Lys} gene cluster (annotated as PA4541.1-3 in PAO1) occurs at its leftward PAO1/PA14 junction, and a 58 bp direct repeat of the 3′ half of the tRNA^{Lys} gene, designated attR, occurs just within its right border, such that it is bounded by 58 bp direct repeats (FIGS. 59A, 59B, and 60A); it contains seven mobility factor genes that encode integrases and transposases, plus four related pseudogenes and direct and inverted repeat sequences (FIG. 64); it appears to have undergone deletions in different P. aeruginosa strains and/or additional insertions have occurred in PA14 (FIG. 61, and described below); and finally, it carries at least 19 virulence factors that occur on genomic islands found in a wide spectrum of other pathogenic bacteria (FIGS. 58 and 63).

Functional organization and predicted ORFs of PAPI-1

Data in FIGS. 34, 60A, and 60B illustrate the highly modular organization and complex origin of PAPI-1, which is inserted in a hypervariable region of the P. aeruginosa genome near the PA4525 pilA gene (Spencer et al., J Bacteriol (2003) 185:1316-1325, and Wolfgang et al., Proc Natl Acad Sci USA (2003) 100:8484-8489). Remarkably, more than 80% of its DNA sequence is unique and shares no similarity with any GenBank sequence. Furthermore, 75 out of its 115 predicted ORFs are unrelated to previously identified proteins or functional domains, and thus cannot be assigned any function by homology.

Conversely, 40 PAPI-1 ORFs translated sequences show homology to proteins from several bacterial species, demonstrating its modular evolution. For instance, 18 PAPI-1 genes display significant homology to pathogenicity-related genes, including a putative type III effector (RL030), a type IVB-like pilus gene cluster (RL077-86), and a chaperone/usher pathway (cup) gene cluster (RL040-44) (FIGS. 34, 60A, and 60B). In this regard, at least two different two-component regulatory systems, RL036-RL037 and RL038-RL039, are included in PAPI-1 (see, for example, FIG. 2). The predicted amino acid sequence of the RL036 and RL038 show high similarity to the RcsC cognate sensor of the RscB of Salmonella enterica subsp. enterica serovar Typhi CT18 based on the presence of a conserved response regulator receiver domain and a histidine kinase-like ATPase domain. The Salmonella RcsB-RcsC regulatory system modulates the expression of invasion proteins, flagellin, and Vi antigen in response to osmolarity (Arricau et al., Mol. Microbiol. (1998) 29: 835-50). In E. coli, targets regulated by the RcsCB system include the exopolysaccharide synthesis genes cps, cell division genes, the osmoregulated gene osmC and genes involved in motility and chemotaxis as well is essential to overcome chlorpromazine-induced stress (Conter et al., J. Bacteriol. (2002) 184: 2850-3). The predicted amino acid sequence of RL039 shows high similarity to the RcsB response regulator of the Salmonella enterica RcsCB system. RL039 contains a response regulator receiver domain, a helix-turn-helix-regulatory motif, and the GerE domain as found in the RcsB. The predicted amino acid sequence of RL037 encodes the PvrR protein, which is involved in Pseudomonas biofilm formation and antibiotic resistence of strain PA14 (Drenkard and Ausubel, Nature (2002) 416: 740-3). The predicted amino acid sequences of ORFs RL040-44 show significant similarity to the cluster of P. aeruginosa CupA gene cluster (PA2128-32) of P. aeruginosa strain PAO1. These genes include components of a chaperone/usher pathway that is involved in assembly of fimbrial subunits in other microorganisms. Such a cluster is also present in the P. aeruginosa strain PAK. Additionally, it has been demonstrated that cups genes are involved in biofilm formation (Vallet et al., Proc Natl Acad Sci USA. (2001) 98: 6911-6). The predicted amino acid sequences of ORFs RL077-86 show high similarity to a type IV biosynthetic pili gene cluster of Salmonella and E. Coli. Pili genes are important for adhesion and biofilm formation. Furthermore, RL011 contains a ParE domain (COG3668), RL012 contains a transcriptional regulator domain (COG3609), RL020 contains a DsbG domain, RL102 contains a ParBC domain, and RL 103 contains an Arc domain (transcriptional repressor). The majority of the remaining PAPI-1 genes that can have a function assigned by homology encode functions related to DNA mobilization, integration and partition activities. Many PAPI-1 predicted proteins are related to sequences found in Salmonella, pathogenic E. coli, Haemophilus somnus, Yersinia pestis, P. aeruginosa, P. syringae, P. fluorescens, Xylella fastidiosa, Burkholderia fungorum and Xanthomonas (FIG. 34). Also, 26 PAPI-1 ORFs translated sequences are similar to predicted proteins on both the 134 kb island of the mammalian pathogen S. enterica (STY4521-4608) (Parkhill et al., Nature (2001) 413:848-852), and the 130 kb island of the phytopathogen X. axonopodis (XAC2171-2286) (da Silva et al., Nature (2002) 417:459-463) (FIGS. 34, 60A, and 60B). Moreover, 21 additional PAPI-1 ORFs show similarity with ORFs from only one of these pathogenicity islands—14 with S. enterica (RL052, 72, 77-79, 81-85) and 7 with X. axonopodis (RL020, 35, 63-65, 67). This complex array of pathogenicity-related genes likely plays a role in the broad host range of PA14.

Interspersed with its ORFs, PAPI-1 carries at least five pairs of direct repeats (DR), a pair of inverted repeats (IR), and an IS sequence also found in P. putida (Nelson et al., Environ Microbiol (2002) 4:799-808) (FIGS. 60A, 60B, 64, and 65). The 63 bp DR1 repeats, which border the entire PAPI-1 element and are part of the tRNA^{Lys} gene, include the 58 bp attR sequence. A hairpin-like structure thought necessary for DNA insertion (van der Meer et al., Arch Microbiol (2001) 175:79-85) occurs downstream of the right DR1, and this sequence might correspond to the actual PAPI-1 integration site, generating the attR and attL sequences. We note that DR 1-like sequences occur in P. aeruginosa strains C and SG17M genomic islands, and in X. fastidiosa (Larbig et al., J Bacteriol (2002) 184:6665-6680).

The 662 bp DR2 repeats encode two proteins of unknown function (RL035 and RL046) and may have served as a DNA integration site. The 15 kb region found between these repeats, which encodes 9 predicted ORFs, includes bacterial genes not previously associated with genomic islands, including two pairs of two-component regulatory systems (RL036-37 and RL038-39). RL037 is the pvrR gene (Drenkard et al., Nature (2002) 416:740-3), while RL039 and RL038 share domains with rcsB and rcsC, which respectively encode a response regulator and a sensor protein of animal and plant pathogenic bacteria (Gottesman et al., Mol Microbiol (1991) 5:1599-606, and Virlogeux et al., J Bacieriol (1996) 178:169′-8). resC is involved in Salmonella virulence (Detweiler et al., Mol Microbiol (2003) 48:385-400). Downstream of these regulatory systems lies a putative fimbrial chaperone-usher gene cluster (RL040-44) that is both related and distinct to cup clusters in P. aeruginosa strains PAO1 and PAK (Vallet et al., Proc Natl Acad Sci USA (2001) 98:6911-6), and to similar clusters from S. enterica and Y. pestis (Parkhill et al., Nature (2001) 413:523-527, 34). We designate the PAPI-1 cluster cupD, since its predicted products are less than 70% identical with those of other cup clusters. cup genes assemble adhesive organelles expressed by many pathogenic bacteria which mediate attachment to epithelial cells (Soto et al., J Bacteriol (1999) 181:1059-71), and contribute to initiation of biofilm formation (Vallet et al., Proc Natl Acad Sci USA (2001) 98:6911-6).

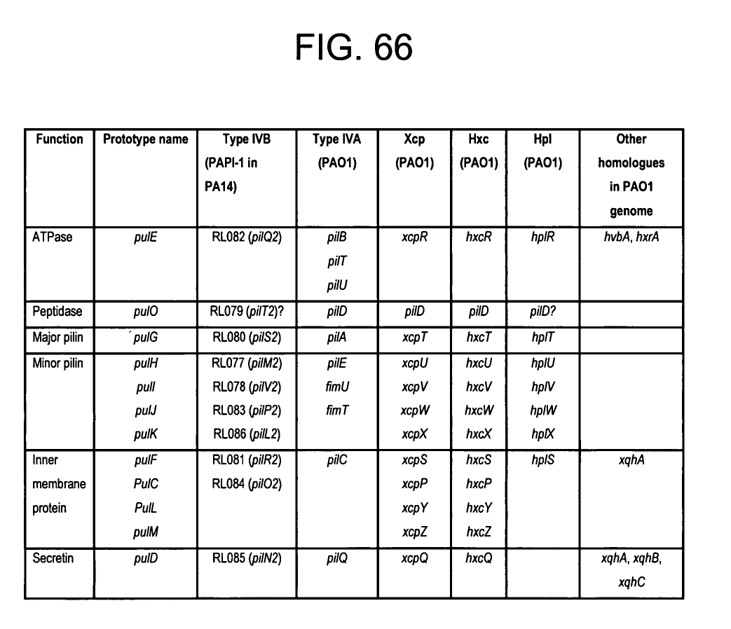

The 248 bp DR3 repeats prescribe a 2.5 kb region of 46.4% G+C, indicating its foreign origin (FIGS. 60A, 60B, and 64). This region contains the RL087-8 genes, which are homologues of PA0984-5 that encode a bacteriocin, pyocin S5, and its immunity protein (Michel-Briand et al., Biochimie (2002) 84:499-510). A pilus biogenesis system (RL077-86) is located just upstream of the left DR3 (FIGS. 60A and 60B). This system resembles type IV group B pili clusters found in other pathogenic bacteria, including the enteropathogenic E. coli bundle-forming pilus, the S. enterica CT18 type IVB pilus, and the V. cholerae toxin-coregulated pilus (Parkhill et al., Nature (2001) 413:848-852, Attridge et al., J Biotechnol. (1999) 73:109-117, Donnenberg et al., Gene (1997) 192:33-38, and Giron et al., (1997) Gene 192, 39-43). Interestingly, the type IV pilus biogenesis machinery is highly homologous to the type II secretion pathways (FIG. 66).

Both DR4 and DR5 consist of two consecutive direct repeats. These repeats are adjacent to the RL092, RL095, RL102, RL109-11 and RL114 ORFs, which are related to plasmid-encoded replication and recombination functions, suggesting that portions of PAPI-1 might be plasmid-derived. In contrast, only two PAPI-1 ORFs (RL 103 and RL110) are phage-related (FIG. 34). Interestingly, the integrase RL002 and the chromosome-partitioning protein Soj RL 115 genes are located at the ends of the island, similar to the P. aeruginosa clone C islands, suggesting that this island may have an intermediate circular form that integrates into tRNA sequences (Kiewitz et al., Microbiology (2000) 146:2365-2373).

Finally, FIGS. 34, 60A, and 60B demonstrate that genomic “shuffling” has also contributed to PAPI-1 organization, as the RL001, RL020, RL087 and RL088 ORFs are closely related to the PA0977-87 genes, which are located on a PAO1 genomic island. RL054-56 and RL113 share homology with the PAO1 genes PA2221-8, which also occur in a region having atypical G+C content.

Overall, PAPI-1 has a highly mosaic structure. It harbors blocks of ORFs related to virulence functions in other human and phytopathogenic bacteria, and ORFs similar to genes found in Archaea and phages, illustrating its diverse foreign origin. The PAPI-1 border regions exemplify this mosaicism. While the right border contains ORFs unrelated to any GenBank sequences, the left border carries ORFs found in Archaea species and in other P. aeruginosa strains (Choi et al., J Bacteriol (2002) 184:952-961). Interestingly, one of these ORFs, RL008, encodes a putative helicase fused to sequences homologous to a PAO1 gene that encodes an unknown function. By mutation analysis, this hybrid ORF encodes a mammalian virulence factor, and thus represents a novel pathogenic function generated via gene fusion.

The highly modular organization of PAPI-1 demonstrates that it was generated by multiple recombination events, as it carries several unrelated genes and gene clusters. Indeed, a large portion of PAPI-1 is similar to ORF clusters found in the genomes of the phytopathogen X. axonopodis pv. citri (da Silva et al., supra), and the human pathogen S. enterica. ser. Typhi (Parkhill et al., Nature (2001) 413:848-852). This region is interrupted by unrelated ORFs located between repeat sequences, suggesting that a fragment homologous to the X. axonopodis and S. enterica gene blocks may have been acquired by P. aeruginosa as a complete DNA fragment, and later interrupted by the insertion of unrelated fragments. Interestingly, one of these secondarily acquired regions, RL036-39, contains two pairs of two-component regulatory systems, which we showed that affect plant and mammalian pathogenesis. Furthermore, these systems may also regulate genes located on PAPI-1 or on the core genome. Acquisition of regulatory systems and virulence genes from other microorganisms may have contributed to the evolution of P. aeruginosa pathogenic variants to thrive in diverse environments. For instance, the PAPI-1 group B type IV pili genes are related to virulence factors that promote pathogen attachment to host cells. Acquisition of these genes could increase P. aeruginosa host-range by promoting its attachment to novel surfaces, such as different epithelial cells.

Characterization of the PAPI-2 Pathogenicity Island

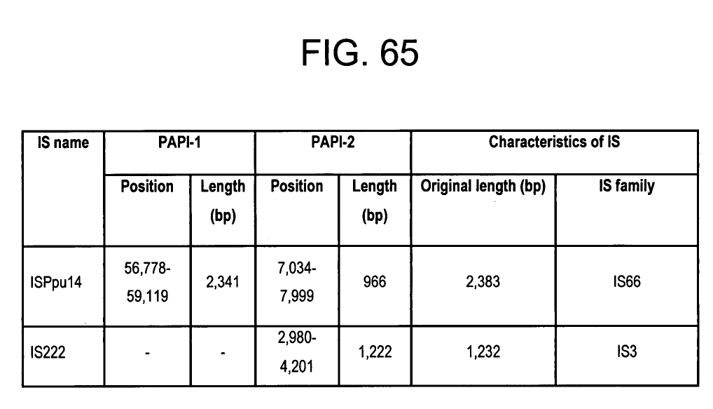

Sequencing of pH44 and pG68, that carry a second copy of RL003, revealed a 10,722 bp region, designated PAPI-2, located near the phnAB genes (FIGS. 59B and 60B), a hypervariable region of the P. aeruginosa genome (Spencer et al., supra, Wolfgang et al., supra, Romling et al., J Mol Biol (1997) 271:386-404). FIG. 60B illustrates the organization of the 15 PAPI-2 predicted ORFs, 7 of which correspond to hypothetical proteins, which by virtue of their location are involved in the pathogenicity of Pseudomonas aeruginosa (FIG. 58). PAPI-2 exhibits features of a genomic island, with a G+C content of 56.4%. It contains multiple predicted mobility functions, including, one integrase gene, four transposase genes, and one related pseudogene (FIG. 58) and has an almost complete IS222 element at its left border as well as a portion of ISPpu14, a putative transposase gene (FIGS. 60B and 65).

Half of PAPI-2 is homologous to PA0977-0987, an 8.9 kb PAO1 genomic island (Kiewitz et al., Microbiology (2000) 146: 2365-2373), which encodes 11 predicted ORFs (FIG. 60B). Unlike PA0977-0987, PAPI-2 is not associated with an attR site but is located at the same position in the P. aeruginosa core genome (FIG. 59B). Furthermore, these two islands share upstream and downstream sequences, and six ORFs (FIGS. 58, 59, and 60). While the PAO1 island unlike PAPI-2, does not contain the entire RS02 integrase gene, it does have an intact tRNA Ys (attL) at its left border and a corresponding 22 bp direct repeat at its right border (attR) (FIG. 59B). The RS03 predicted product shares homology with the N-terminus of that of RL003, the 33A9 locus (FIG. 58). Interestingly, the 2.5 kb left end of PAPI-2 is identical to the 2.5 kb left end of PAPI-1, from the tRNA^{Lys} gene to RL003 and RS03, respectively (FIGS. 60A and 60B). Finally, the PAO1 pyocin genes PA0984-85 are replaced in PAPI-2 by the cytotoxin exoU gene and its chaperone spcU (RS14-15). exoU is a type III effector that plays an important role in pathogenesis (Miyata et al, Infect Immun (2003) 71:2404-13). Its presence on PAPI-2 defines this block as a pathogenicity island.

PAPI Island ORFs Encode Novel Pathogenicity-Related Functions

We generated and analyzed 23 mutant strains (FIG. 63), including 10 non-polar deletions and 13 TnphoA transposon insertion mutants, to assess whether the PAPI-1 and PAPI-2 ORFs that encode hypothetical/unknown functions promote P. aeruginosa pathogenesis. None of the mutants was defective for growth in liquid culture or for the extracellular production of pyocyanin and pyoverdine and for protease activity. Since some of the known PAPI-1 genes are involved in adhesion and/or motility, we also evaluated the mutants for colony morphology, in vitro adhesion, and swimming, twitching, and swarming motilities. All the mutants behaved like the wild-type parent, indicating that these activities do not depend on the mutated ORFs, under our experimental conditions.

Virulence was assessed in plants and animals using the Arabidopsis leaf infiltration and the mouse thermal injury models (Rahme et al., Proc Natl Acad Sci USA (1997) 94:13245-50) as shown in FIG. 63. To assess animal mortality, mice were infected with 5×10⁵ bacterial cells. Eight to sixteen mice were used per experiment. To assess plant pathogenicity, Arabidopsis leaves were inoculated with 3.3×10⁵ bacterial cells and assayed three days post-infection for bacterial CFU/cm². Four different leaves were sampled. All experiments were performed twice. Statistical significance for mortality data and bacterial growth in Arabidopsis leaves were determined by the t test and shown in bold. Differences between groups were considered statistically significant at p<0.05. Statistically different comparisons are shown in bold. FIG. 63 shows that 20 of the 23 mutants exhibited attenuated virulence phenotype in at least one of the hosts, with 12 attenuated in both. Importantly, 15 of these mutants correspond to novel genes and one to a known gene (pvrR) but not previously shown to be involved in virulence. Of the mutated ORFs, RL016, RL022, and RL029 occur within a large region (RL012-30) found in several other phytopathogenic bacteria, and RL036-37 and RL038-39 encode two-component regulatory systems, suggesting that pathogenicity activities regulated by these systems are evolutionarily conserved.

Presence of PAPI-1 in Other P. aeruginosa Clinical Isolates

We used 11 hybridization probes spanning PAPI-1 to assess its occurrence in 14 P. aeruginosa pathogenic strains, 12 of which were isolated from CF patients (FIG. 61) (Wolfgang et al., supra, and Liang et al., supra). These probes were nucleic acid fragments of PAPI-1 (SEQ ID NO: 2) and included nucleotides 2323-4185 of SEQ ID NO: 2 (SEQ ID NO: 283), nucleotides 3699-5161 of SEQ ID NO: 2 (SEQ ID NO: 284), nucleotides 11351-12180 of SEQ ID NO: 2 (SEQ ID NO: 285), nucleotides 25562-26456 of SEQ ID NO: 2 (SEQ ID NO: 286), nucleotides 35321-36307 of SEQ ID NO: 2 (SEQ ID NO: 287), nucleotides 40536-41653 of SEQ ID NO: 2 (SEQ ID NO: 288), nucleotides 61179-63605 of SEQ ID NO: 2 (SEQ ID NO: 289), nucleotides 74931-76115 of SEQ ID NO: 2 (SEQ ID NO: 290), nucleotides 84920-86620 of SEQ ID NO: 2 (SEQ ID NO: 291), nucleotides 103068-104554 of SEQ ID NO: 2 (SEQ ID NO: 292), or nucleotides 104797-105543 of SEQ ID NO: 2 (SEQ ID NO: 293).

While CF1, CF3, CF4, CF5, CF27, CF28, CF30 and CF32 did not hybridize with any of the probes used, PA037, CF2 and CF6 hybridized with all of them, suggesting that these strains carry the entire 108 kb PAPI-1 island. In contrast, other strains hybridized to a subset of probes. CF26 appears to carry the complete island, except for the region found between the DR2 sequences, while CF29 only carries its leftward half. Both PAK and PAO1 contain only a small segment of PAPI-1, with PAK carrying its left end, and PAO1 a 1.7 kb region that harbors the pyocin S5 and immunity genes.

The fact that several PAPI-1 and PAPI-2 genes have known relatives in the genomes of plant, soil, animal, and human-associated bacterial species is not surprising, as P. aeruginosa inhabits soil and water environments, and is associated with several hosts. It is likely that during its evolution P. aeruginosa has encountered a diverse array of bacterial species that have donated, and continue to donate, foreign DNA fragments. In turn, these fragments have affected the evolution of P. aeruginosa pathogenic variants to colonize even more environments. Presumably, this gene flow is bi-directional, such that P. aeruginosa virulence genes have spread to other bacterial species, to generate novel virulent strains in these species as well.

PAPI-1 and PAPI-2 mutational analysis demonstrate that both islands carry genes that allow this pathogen to thrive on evolutionary diverse hosts, including plants (Arabidopsis) and mammals (mouse). Indeed, of the 23 ORFs mutated here, 19 encode functions necessary for plant or mammalian virulence, with 12 required for “wild-type” virulence in both hosts. Although the majority of these genes encode products of unknown function, their presence in P. aeruginosa clinical isolates, including those from CF patients, may be important for fitness and survival. The characterization of these novel pathogenicity factors may provide insights into broad host pathogenic and defense mechanisms. Completion of the PA14 genome sequence may also result to the identification of additional PAPI blocks and novel virulence genes.

The above studies were performed using the following materials and methods.

Clones Containing Nucleic Acids of the Invention

Exemplary clones containing nucleic acids of the invention are described in Table 1. For example, ORF7 is in p148. To generate these cosmid clones shown in FIGS. 62A and 62B, Pseudomonas aeruginosa genomic DNA fragments were inserted into the pRR544 vector.

TABLE 1
Clones containing nucleic acids of the invention

| | | |
|---|---|---|
| Cosmid Clones | Size of the insert | Island sequence ref # |

Pathogenicity Island-1, PAPI-1 (Big island)

| | | |
|---|---|---|
| pI48 | 42000 bp | 01-23929 bp |
| pG22 | 40000 bp | 11824-52379 bp |
| pSK91 | 39000 bp | 48048-87433 bp |
| pSK24 | 40000 bp | 79084-108759 bp |

Pathogenicity Island-2, PAPI-2 (Small island)

| | | |
|---|---|---|
| PH44 | 40000 bp | 01-11024 bp |

Deposit

Pseudomonas aeruginosa strain UBCPP-PA14 has been deposited with the American Type Culture Collection on Mar. 22, 1995, and bears the accession number ATCC No. 55664. Cosmid clones p148, pG22, pSK91, pSK24 and PH44 have been deposited with the American Type Culture Collection, and bear the accession numbers ATCC No. PTA-4768 (deposited Oct. 25, 2002), PTA-4766 (deposited Oct. 25, 2002), PTA-4666 (deposited Sep. 13, 2002), PTA-4767 (deposited Oct. 25, 2002), and PTA-4664 (deposited Sep. 13, 2003), respectively. Applicants acknowledge their responsibility to replace these clones should they lose viability before the end of the term of a patent issued hereon, and their responsibility to notify the American Type Culture Collection of the issuance of such a patent, at which time the deposit will be made available to the public. Prior to that time the deposit will be made available to the Commissioner of Patents under terms of CFR § 1.14 and 35 USC § 112.

Strains, Plasmids, and Media

All P. aeruginosa parental strains are human isolates (Rahme et al., Science (1995) 268:1899-902, Wolfgang et al, supra, and Liang et al., supra). The TnphoA mutant 33A9 has been previously described (Rahme et al., Proc Natl Acad Sci USA (1997) 94:13245-50). The PA14 genomic cosmid library, constructed in pJSR (Rahme et al., (1995) Science 268:1899-902), was grown in E. coli VCS257, and subcloned in DH5α.pRK2013 and pEX18Ap (Hoang et al., Gene (1998) 212:77-86) served respectively as the P. aeruginosa conjugation helper plasmid and marker exchange suicide vector. Bacteria were grown in LB plus 100 μg/ml ampicillin (E. coli), 100 μg/ml rifampicin (PA14), and 250 μg/ml carbenicillin (PA14 transconjugants).

DNA Methods and Library Construction

Probes were labeled with [³²P]-dCTP (NEN) using Rediprime II (Amersham Pharmacia Biosciences). Genomic, cosmid and plasmid DNA extractions followed standard procedures (Ausubel et al., (1998) John Wiley and Sons, New York). To construct the PA14 cosmid library, a 30-50 kb partial Sau3AI digest of total PA14 DNA was size-fractionated in a 10-40% sucrose gradient, cloned into the BamHI site of pJSR, and packaged using Gigapack III XL (Stratagene).

PA14 Mutants

Non-polar deletions in eleven PAPI-1 ORFs and one PAPI-2 ORF were generated by PCR: 1.0 to 1.6 kb 5′ and 3′ segments were amplified from target PA14 genomic or cosmid DNA, and each amplicon, which included the first or last 10-20 amino acids of the target ORF, plus an engineered restriction site, were ligated into pEX18Ap, to produce replacement plasmids. Non-polar mutants were generated in PA14 via homologous recombination by sucrose resistance selection, and confirmed by hybridization.

Twelve TnphoA transposon insertion mutants of PA14 were obtained from a partial library currently being developed, which, when completed, will include transposon-insertion mutants of all non-essential PA14 ORFs. Access to mutants and information about this library is currently available via a web interface. (MGH-Parabiosys: NHLBI Program for Genomic applications, Massachusetts General Hospital and Harvard Medical School, Boston, Mass.; http://pga.mgh.harvard.edu/cgi-bin/pa14/mutants/retrieve.cgi).

Plant and Mouse Pathogenicity Studies

Mouse mortality and Arabidopsis thaliana (ecotype Col-1) plant infection studies were as described (Rahme et al., Science (1995) 268:1899-902).

DNA Sequencing and Annotation

The nucleotide sequences of the PA14 pI48, pG22, pSK91, pSK24, pF62 and pH44 cosmids were determined by shotgun sequencing. Cosmid fragments subcloned into pBluescript SK (−) and pDN19 were sequenced by primer walking to cover gaps. Individual reads were aligned and assembled using DNAstar and CAP (http://pbil.univ-lyon1.fr/cap3.html). The sequence was compared to the PAO1 annotated genome (http://www.pseudomonas.com) (Stover et al., supra.). tRNA genes were identified using tRNA-sacn-SE (http://www.genetics.wustl.edu/eddy/tRNAscan-SE). ORFs were predicted using GeneMark.hmm (http://opal.biology.gatech.edu/GeneMark/gmhmm2_prok.cgi; (Lukashin et al., Nucleic Acids Res (1998) 26:1107-1115) and ORF finder http://www.ncbi.nlm.nih.gov/gorf/gorf.html). Each predicted ORF of greater than 200 bp was analyzed for homologies and conserved motifs using BlastN, BlastP and BlastX. A full array of parameters was used. PSORT (http://psort.nibb.acjp/form.html) and TMpred (http://www.ch.embnet.org/software/TMPRED_form.html) were respectively used to predict cellular localization and transmembrane regions.

Characterization of ORF7

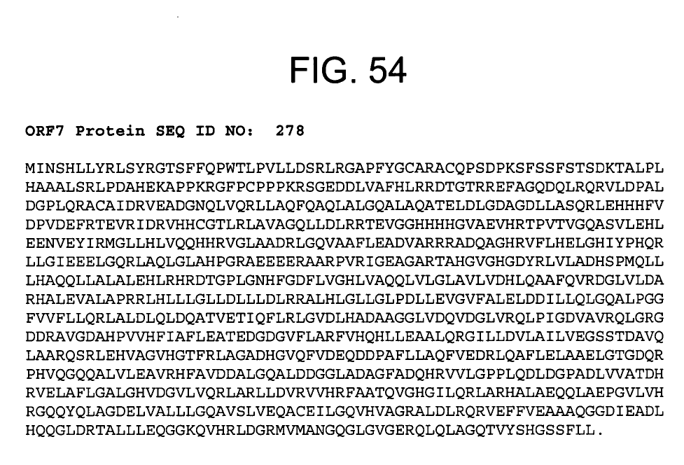

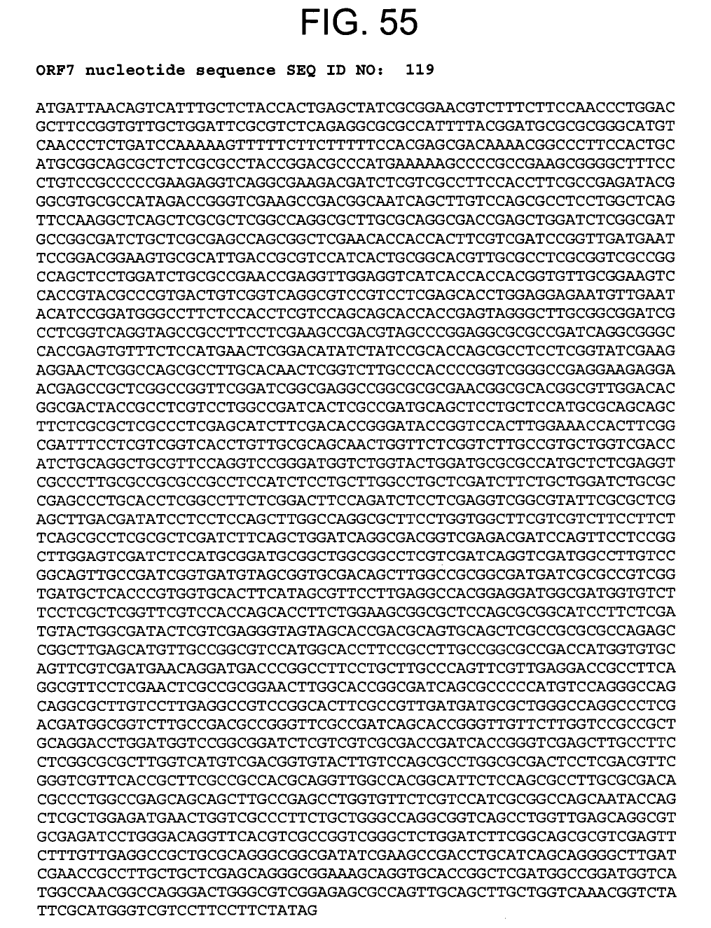

The amino acid sequence (SEQ ID NO: 278) and nucleotide sequence (SEQ ID NO: 119) of ORF7 are shown in FIGS. 54 and 55, respectively. The sequence of the predicted translation product of ORF7 (found in PAPI-1) revealed the presence of a Arg-Gly-Asp (RGD) tripeptide sequence, which is a characteristic motif found in eukaryotic proteins that bind to host cell surface integrins and are involved in bacterial adherence. Site-directed mutagenesis was performed to convert the Arg-Gly-Asp (RGD) tripeptide into Trp-Ile-His (W1H). After introducing the mutation into the PA14 chromosome via homologous recombination, a burned mouse model and a neonatal lung infection model were used to test the function of the RGD motif. The ORF7 RGD mutants had reduced virulence, thus indicating that this ORF is involved in virulence.

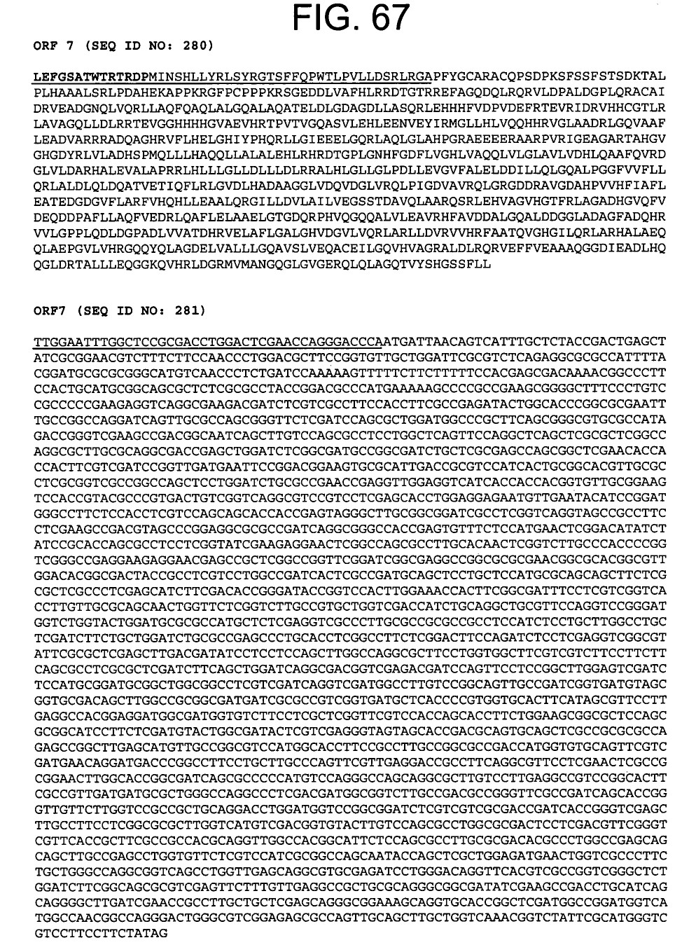

We have further found an alternative translational start codon for ORF7 (TTG) is predicted by the Glimmer software (http://glimmer.sourceforge.net/), adding 14 aminoacids to the above sequence. The updated sequence (SEQ ID NO: 280) is shown in FIG. 67, with the 14 first aminoacids highlighted. Also shown is the corresponding nucleotide sequence (SEQ ID NO: 281). In addition, using the software “SMART” (http://smart.embl-heidelberg.de/) a signal sequence of 48 aminoacids (underlined) is predicted in the N-terminal portion of the protein, indicating that the ORF7 protein may be translocated through the inner membrane.

The translational start site identified was confirmed by constructing a lacZORF7 translational fusion. The first eleven ORF7 codons were fused to the reporter gene lacZ and cloned into a plasmid vector. The ORF7 protein is translated in both Escherichia coli and Pseudomonas aeruginosa. The fact that the ORF7 gene encodes for a protein was further confirmed by introducing a nonsense mutation in its 42^{nd} codon of the ORF7 and a chromosomal mutation was generated without interfering with the overlapping c/pB gene. This mutant strain, denominated ORF7stop, exhibited attenuated virulence in the plant Arabidopsis thaliana. Virulence may further be tested in the mouse burn model. Thus, our results demonstrate that the ORF7 DNA is transcribed and translated in vivo.

ORF7 Promoter Region Analysis

The mapping of the transcriptional start sites of ORF7 was carried out by primer extension experiments. Synthetic oligonucleotides (18-mer) complementary to the mRNA was 5′ end labeled with [γ-³²P]ATP and polynucleotide kinase and hybridized to 50 μg of total RNA isolated with RNeasy miniprep kit (Qiagen) from PA14 mid-exponential cultures grown at 37° C. Annealing was carried out at 50° C. overnight in 8 mM piperazine-N,N′-bis (2-ethanesulfonic acid) buffer pH 6.4 containing 80 M NaCl, 0.2 mM EDTA and 80% formamide. The nucleic acids were ethanol precipitated and resuspended in MMLV-RT (Moloney murine leukemia virus reverse transcriptase) reaction buffer (Amersham) containing 1 mM each dNTP and 40 u of RNAsin (Promega). The annealed primer was extended at 37° C. for 90 min using 300 U of MMLV-RT (Amersham). RNA was digested for 30 min at 37° C. by the addition of 23 μg/ml RNase A, and the extended products were analyzed by electrophoresis on denaturing sequencing gels followed by autoradiography.

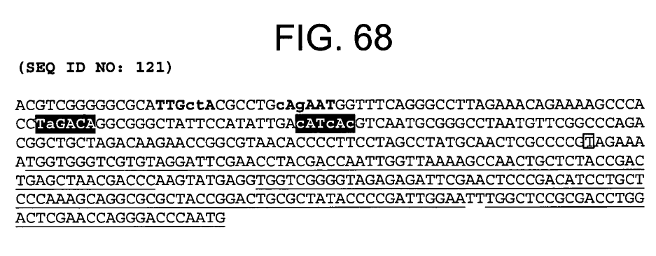

These experiments showed two putative transcription start sites for ORF7, originating from the region inside the large pathogenicity island, as shown in FIG. 68.

Presence of the ORF7 Regulatory Region in other Pseudomonas aeruginosa Strains

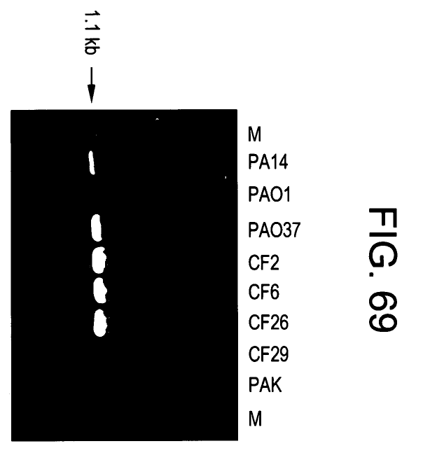

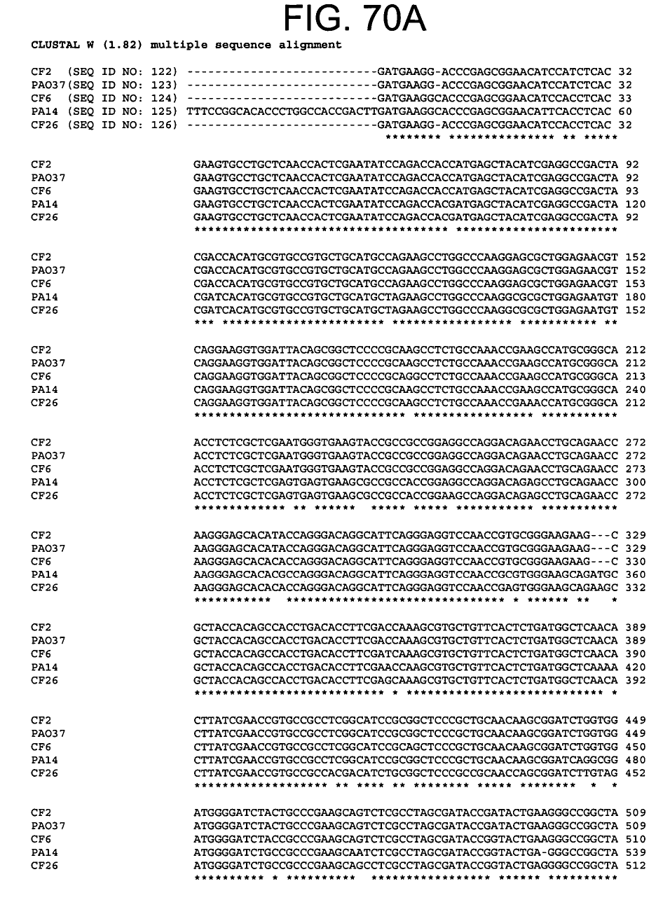

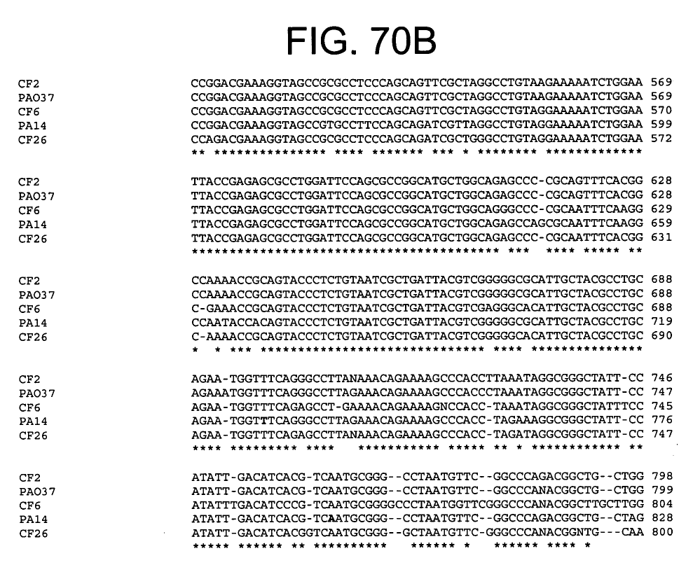

In order to determine whether ORF7 is also present in the genome of other P. aeruginosa strains besides PA14, we carried out PCR reactions using as one primer the sequence found upstream of the promoter region [orf7 (1), 5′-CCC CAA GCT TGC ACA CCC TGG CCA CCG ACT T-3′]. The second primer is designed based on sequences found within the ORF7 coding region [orf7(2), 5′-TGA GAC GCG GAT CCA GCA ACA]. Total DNA isolated from the P. aeruginosa strains PA14, PAO1, CF2, CF6, CF26, CF 29, and PAK was used as a template. As illustrated in the FIG. 69, a band of the expected 1.1 kb size was obtained from the PA14, PA037, CF2, CF6 and CF26 total DNA. For verification, all PCR products were sequenced (except PA037) and the sequences were aligned by Clustal W at http://www.ebi.ac.uk/clustalw/alignment software. As shown in FIG. 70, all conserved nucleotides are indicated by *. The PA14 transcription start sites that were determined experimentally are boxed.

Protein Interactions with ORF7

To study the role of ORF7 in more detail, a standard two-hybrid approach was used. The yeast two-hybrid system is a simple screening method for protein-protein interactions using the transcriptional activation of secondary reporters as a readout. Briefly, the first protein is expressed as a translational fusion to a DNA-binding domain (DBD) of known binding site specifity, and the interacting protein is expressed as a translational fusion to a transcriptional activation domain (AD). One or more reporter genes are transcriptionally dependent on activation through the cognate binding site or the DBD. Both fusions are introduced into yeast cells, and the interaction of both protein fusions (the DBD fusion“bait” and the AD fusion “prey,” respectively) positions the activation domain in proximity to the reporter gene and activates transcription of the reporter gene. In order to identify potential interacting partners, ORF7 was used as a bait to screen a human lung library.

Cloning of ORF7 from Genomic DNA

The ORF7 gene from Pseudomonas aeruginosa PA14 strain was PCR amplified from genomic DNA using Pwo DNA-polymerase (Roche) and the following oligonucleotides; #63 forward (5′-GCGGATCCCCATGATTAACAGTCATTTG-3′) and #64 reverse (5′-CCGTGATCACTATAGAAGGAAGGACGAC-3′). The PCR reaction was set up according to the manufactures instructions as follows (Table 2).

TABLE 2

| | |
|---|---|
| 10xPwo-Puffer with MgSO₄ | 5.00 μl |
| 10 mM dNTPs | 1.00 μl |
| oligo forward 100 pmol/μl | 0.25 μl |
| oligo reverse 100 pmol/μl | 0.25 μl |
| genomic DNA | 2.00 μl |
| Pwo DNA-polymerase | 0.50 μl |
| 5 U/μl | |
| DMSO | 5.00 μl |
| Water | 41.0 μl |
| Total | 50.0 μl |

The following PCR program in a Perkin Elmer ThermoCycler 9600 was used: 30 seconds at 94° C.; 30 cycles in which each cycle included 30 seconds at 94° C., 30 seconds at 50° C., 72° C., and 7 minutes at 72° C. The product of the PCR reaction was separated by agarose gel electrophoresis and extracted using a gel extraction kit (QIAGEN).

Construction of pGBKT7-ORF7

The gel extracted PCR product of ORF7 was digested with the restriction endonucleases BamHI (NEB) and BclII (NEB). This fragment was subsequently ligated into the BamHI site of vector pGBKT7 (Clontech), which was treated with calf intestinale phosphatase (CIP) to reduce background religation of the vector itself. The ligation reaction mixture was transformed into TOP10 competent cells, and DNA was prepared using the DNA mini prep protocol (QIAGEN). The correct clone was first analyzed by digestion with restriction endonucleases and then verified by sequencing.

Two-Hybrid Screen with a Human Lung Library

A human lung cDNA library (Clontech, #HL4044AH) was used in the two-hybrid screen. This library was made from RNA of two normal whole lungs pooled from two females, and the cDNA was cloned via an adaptor in XhoI/EcoRI of pACT2. The average cDNA size is 2.0 kb, and the size range was from 0.4-4.0 kb.

The human lung library was amplified to obtain enough DNA to perform the two-hybrid screen. First, the titer of the library was estimated by striking different aliquots of the library containing bacteria in different dilutions on LB plates with ampicilin (final concentration 100 μg/ml) and incubating them for 24-36 hours at 30° C. The number of colonies indicated a titer of ˜1×10⁹ colonies per ml. Then, the number of plates needed for amplification was calculated as follows: 3.3×10⁶ independent clones×3=9.9×10⁶ clones to be screened. Since only 85% of the colonies contained an insert, 111.6×10⁶ clones have to be screened. By spreading out 20,000 colonies per 15 mm plate, the library was amplified using 580 plates (11.6×10^{6/20,000=580}). The bacteria were spread onto 580 LB/ampicilin plates and incubated overnight at 30° C. The colonies from each plate were collected by adding 5 ml LB medium and scraping the cells using a cell scraper. The collected cells were incubated for 3.5 hours at 30° C. The cells were harvested and frozen in aliquots. Approximately, 40 ml bacterial pellets were resuspended in 800 ml P1 buffer, and DNA was isolated using the GigaprepKit (QIAGEN) according to the manufacturer's instructions.

An aliquot of the amplified library was checked by PCR for the presence of three different genes (i.e., human β-actin, human transferrin receptor, and human glyceraldehyde 3-phosphate dehydrogenase (G3PDH)) present in the original library. All three genes were amplified out of the re-amplified library, demonstrating that the amplification did not notably affect the relative distribution of the genes. For the amplification of human β-actin, 5′primer #20 β-actin (5′-ATCTGGCACCACACCT TCTACAATGAGCTGCG-3′ and 3′primer # 19 β-actin (5′-CGTCATACTCCTGCTTG CTGATCCACATCTGC-3′ were used. For human transferrin receptor, 5′primer (5′-CCACCATCTCGGTCATCAGGATTGCCT-3′ and 3′primer (5′-TTCTCATGGAAGCTATGGGTATCACAT-3′ were used. For human glyceraldehyde 3-phosphate dehydrogenase (G3PDH), 5′primer (5′-TGAAGGTCGGAGTCAAC GGATTTGGT-3′ and 3′primer (5′-CATGTGGGCCATGAGGTCCACCAC-3′ were used. The library transformation and the two-hybrid analysis were performed according the manual MATCHMAKER Two-Hybrid System 3 (#K1612-1, Clontech).

Isolation of Plasmid DNA from Yeast

This procedure for plasmid isolation was adapted from the QlAprep Spin Miniprep Kit protocol by QIAGEN. A single colony was inoculated into 2-5 ml of the appropriate selective media, and the culture was grown for 16-24 hours at 30° C. The cells were harvested by centrifugation for five minutes at 5000×g and resuspended in 250 μl Buffer P1 containing 0.1 mg/ml RNase A. The cell suspension was transferred to a 1.5-ml microfuge tube. Acid-washed glass beads (50-100 μl, Sigma G-8772) were added and vortexed for five minutes. The beads were allowed to settle. The supernatant was transferred to a fresh 1.5-ml microfuge tube. Lysis buffer P2 (250 μl) was transferred to the tube and inverted gently 4-6 times to mix. The mixture was incubated at room temperature for five minutes. Neutralization buffer N3 (350 μl) was added to the tube and inverted immediately but gently 4-6 times. The lysate was centrifuged for 10 minutes at maximum speed in a tabletop microcentrifuge (13,000 rpm or 210,000×g). The cleared lysate was transferred from a QIAprep spin column by decanting or pipetting, and centrifuged for 30-60 seconds (13,000 rpm or over 10,000×g). The flow-through was discarded. The QlAprep spin column was washed by adding 0.75 ml of Buffer PE and centrifuging 30-60 seconds (13,000 rpm or over 10,000× g). The flow-through was discarded, and the sample was centrifuged for an additional minute to remove residual wash buffer (13,000 rpm or over 10,000×g). The QlAprep column was placed into a clean 1.5-ml microfuge tube. To elute DNA, 25 μl of Buffer EB (10 mM Tris CI, pH 8.5) or H₂O was added to the center of each QIAprep spin column, incubated for one minute, and centrifuged for one minute. Typically, 2-5 μl of the eluate was transformed in E. coli to obtain at least 5-20 colonies. These colonies were then inoculated to isolate plasmid DNA.

Subcloning of Human Lung Genes into pGADT7

The fragments containing the reading frame of the human lung genes were subcloned into the pGADT7 vector (Clontech) using the following strategy. The insert was PCR amplified by using the forward primer # 13 (5′-CTATTCGATGATG AAGATACCCCACCAAACCC-3′) and the reverse primer #89 (5′-ACTTGCGGGG TTTTTCAGTATCTACGAT-3′). The amplified DNA was then digested with the restriction endonuclease SfiI and ligated in frame with SfiI/SmaI digested pGADT7. The reverse primer was phosphorylated allowing the treatment of pGADT7 with CIP. Only the plasmids pS136 and pS137 were cloned differently since the inserts contained an internal SfiI-site. The insert was PCR amplified using the forward primer #88 (5′-GGGATCCCGAATTCGCGGCCGCGTCGAC-3′) and the reverse primer #89. The amplified DNA was then ligated in frame in the SmaI site of pGADT7. All plasmids were verified by sequencing (Table 3).

TABLE 3

| # | plasmid name |
|---|---|
| pS131 | pACT2-human lung gene 1C3 #2 |
| pS132 | pACT2-human lung gene 1F8 #8 |
| pS133 | pACT2-human lung gene 4C6 #47 |
| pS134 | pACT2-human lung gene 4E7 #56 |
| pS135 | pACT2-human lung gene 4F8 #59 |
| pS136 | pACT2-human lung gene 4F10#60 |
| pS137 | pACT2 human lung gene 4H8 #63 |
| pS138 | pACT2-human lung gene 5A1 #65 |
| pS139 | pACT2-human lung gene 5E7 #80 |
| pS140 | pACT2-human lung gene 5G1 #86 |
| pS420 | pGADT7-human lung gene 1C3 #2 |
| pS421 | pGADT7-human lung gene 1F8 #8 |
| pS422 | pGADT7-human lung gene 4C6 #47 |
| pS423 | pGADT7-human lung gene 4E7 #56 |
| pS424 | pGADT7-human lung gene 4F8 #59 |
| pS425 | pGADT7-human lung gene 4F10#60 |
| pS426 | pGADT7-human lung gene 4H8 #63 |
| pS427 | pGADT7-human lung gene 5A1 #65 |
| pS428 | pGADT7-human lung gene 5E7 #80 |
| pS429 | pGADT7-human lung gene 5G1 #86 |

Analysis of Identified Human Lung Genes

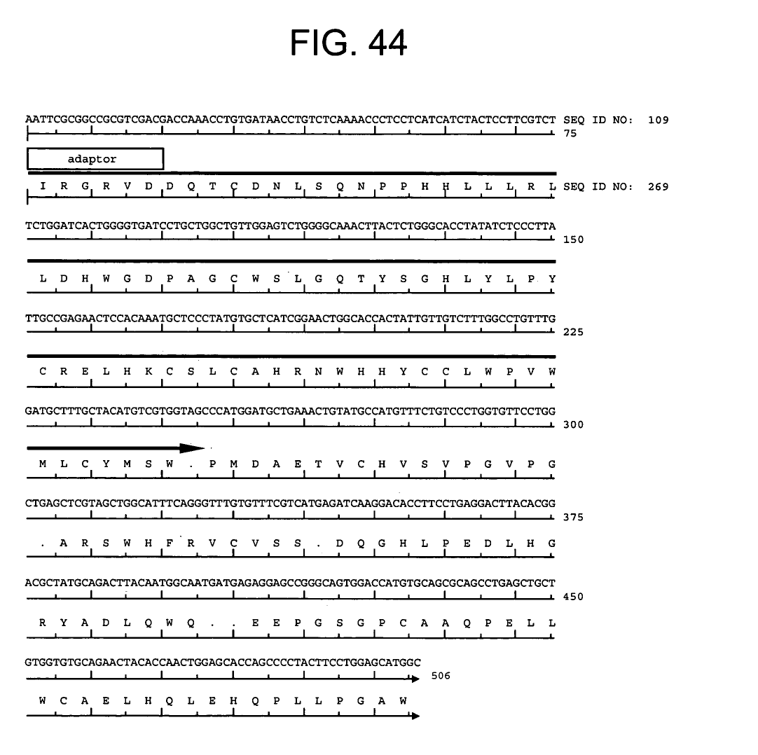

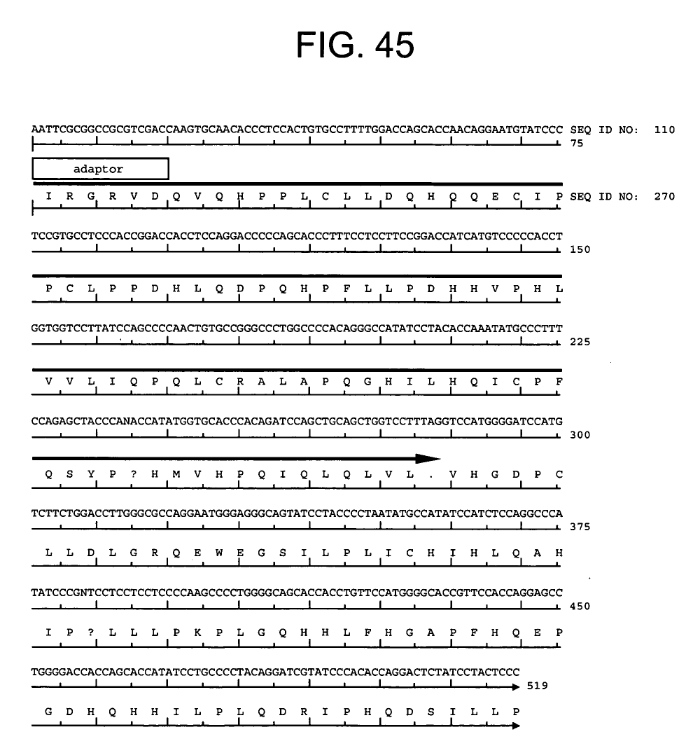

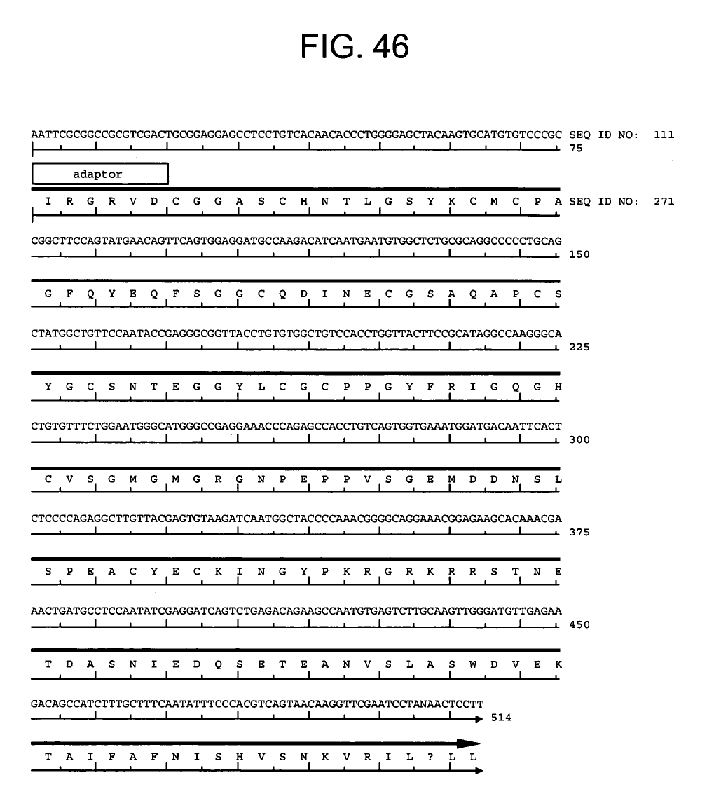

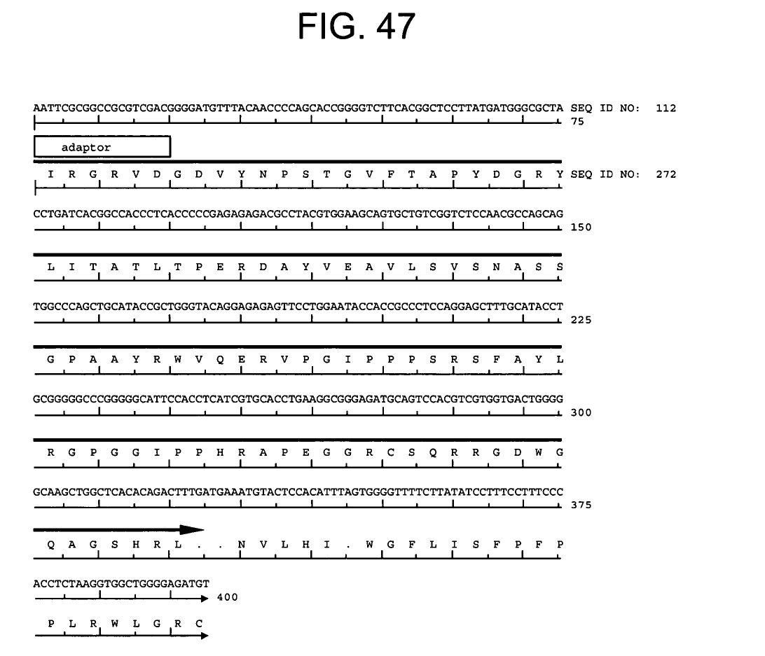

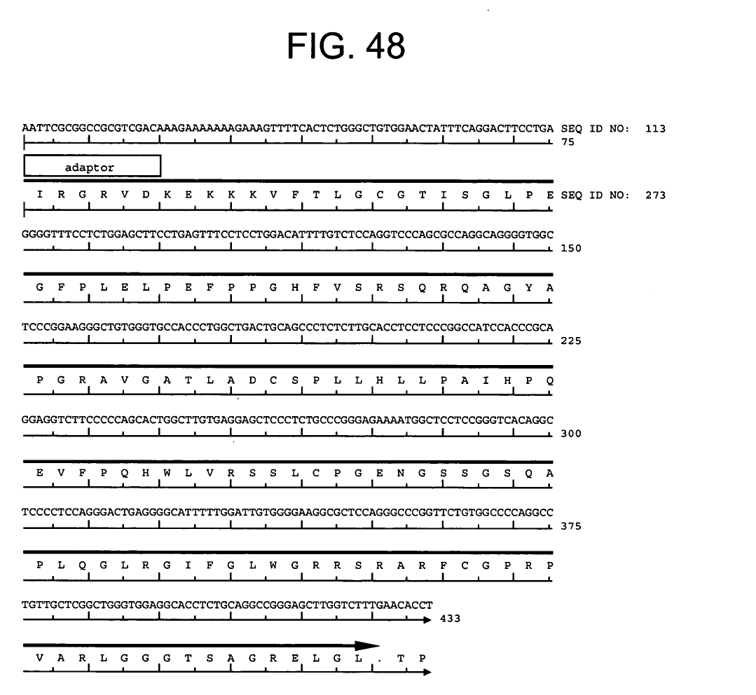

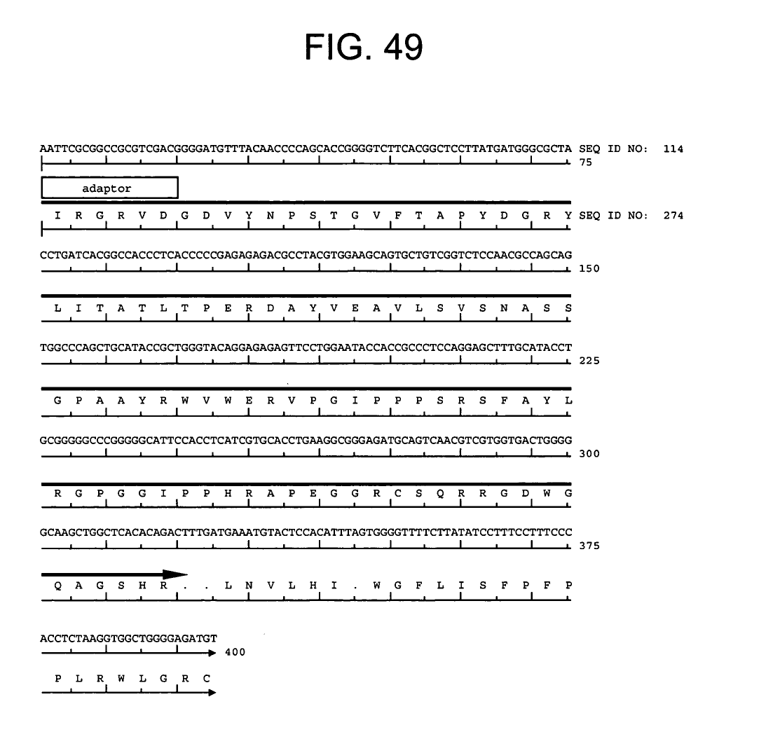

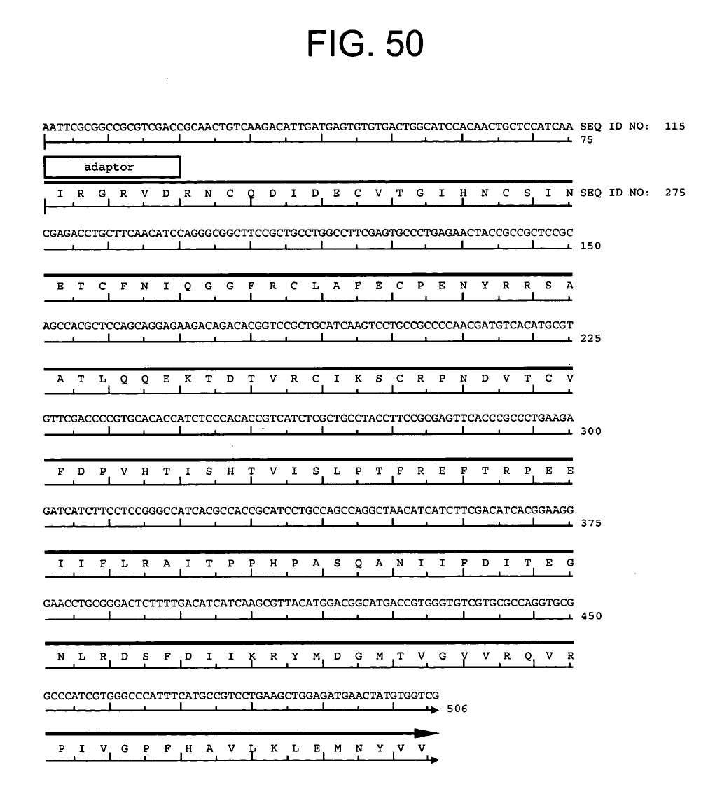

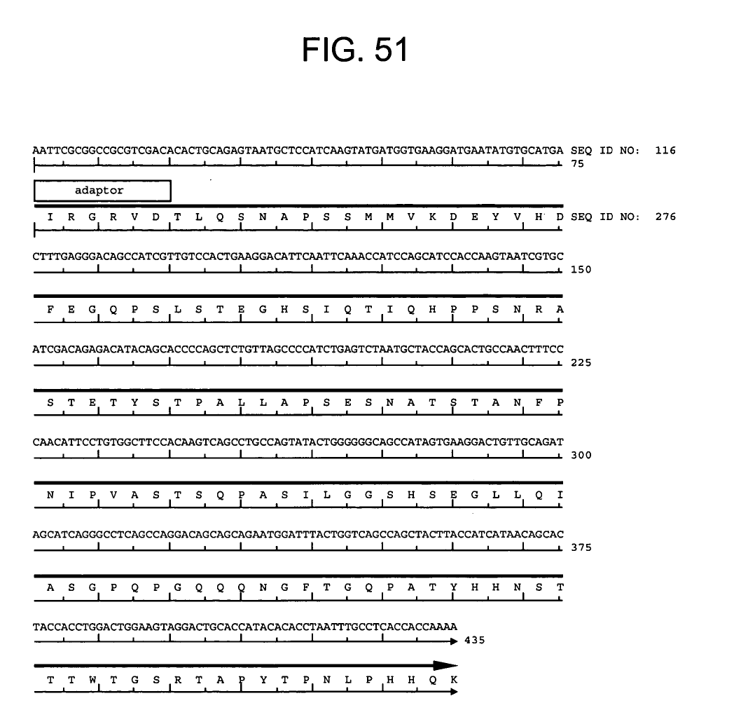

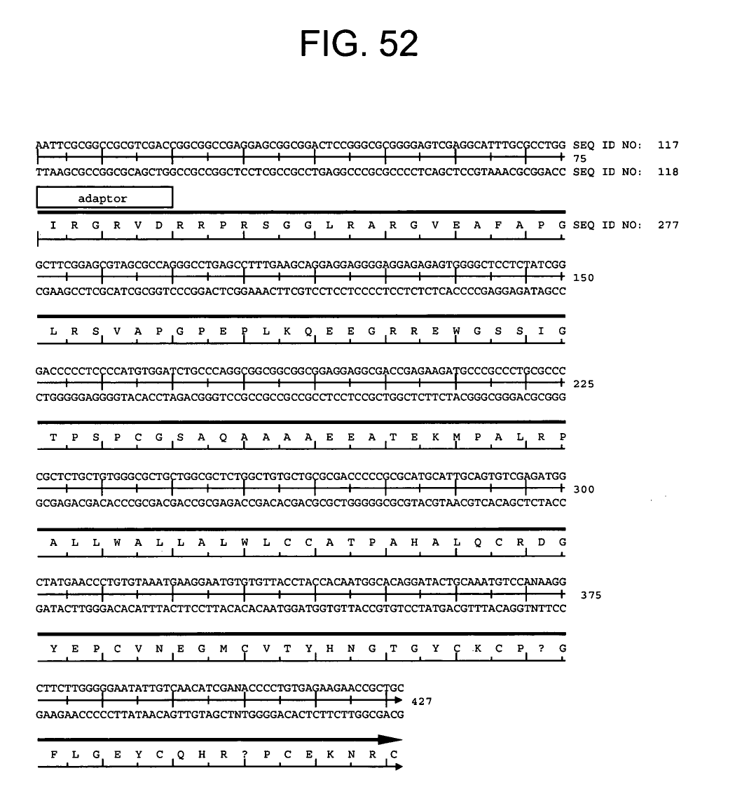

To identify the clones isolated from the human lung library, the respective plasmid DNA was isolated, and the DNA sequence was determined by sequencing. These sequences were used to search for homologous genes using the Bioscout program (LION, Heidelberg). The nucleotide (SEQ ID NO: 109-118) and amino acid sequences (SEQ ID NO: 269-277) of the identified human lung genes are shown in FIGS. 44-52.

Western Blot Analysis of Yeast Cells

Yeast cells were incubated in selective media to ensure the presence of plasmids and grown to an optical density of OD₆₀₀=1.0 (2×10⁷ Zellen/ml). Cells (4 OD units) were centrifuged for five minutes at 2,500 rpm (1,430×g) at 4° C. The supernatant was carefully discarded and any residual media was completely removed. The pellet was resuspended in 0.5 ml 0.25M NaOH/1% 2-mercaptoethanol and incubated on ice for 10 minutes. Ice-cold 50% trichloracetic acid was added and vortexed. The reaction was further incubated for 10 minutes on ice and subsequently centrifuged for 10 minutes at 14,000 rpm (15,800×g) and 4° C. The pellet was washed with 1 ml ice-cold acetone and shortly dried. SDS sample buffer (95 μl) and 5 μl 1 M tris, pH 8.0 was added. Before loading the samples onto an SDS gel, the proteins were denatured by boiling for five minutes at 95° C.

For the detection of the fusion proteins or for the co-immunoprecipitation experiments (see next section) the following antibodies were used: anti-c-Myc (Clontech, mouse monoclonal, final concentration 2.0 μg/ml), anti-HA (Roche, 12CA5, mouse monoclonal, final concentration 0.5 μg/ml), anti-Gal4 DB (Clontech, mouse monoclonal, final concentration 0.5 μg/ml), and anti-Gal4 AD (Clontech, mouse monoclonal, final concentration 0.4 μg/ml), and anti-Maus Ig antibody developed in goat (Amersham). The blots were developed using the ECL^{PLUS} system (Amersham) and visualized with the Image station 440CF (Kodak).

Develop Antibodies Against ORF7

Peptide antibodies were generated by Eurogentec against the following peptide sequences in a rabbit: EPO11500 Peptid AS 73-86 (H₂N-CPDAHEKAPPKRGFP-CONH₂) and EPO11501 Peptid AS 43-58 (H₂N-CQPSDPKSFSSFSTSD-CONH₂). These antibodies were able to detect ORF7 in yeast cells.

Coimmunoprecipitation Experiments to Confirm the Interaction in an Independent Assay

The protein interaction detected through an in vivo two-hybrid screen was confirmed by an in vitro biochemical assay. The DNA-binding domain (DBD) containing vector pGBKT7 has a T7 promoter and a c-myc epitope tag allowing the direct application of this vector in an in vitro transcription/translation reaction. The human lung genes were subcloned into the pGADT7 vector to in vitro transcribe/translate a fusion with the HA-epitope tag. Because the T7 promoters and epitope tags are located downstream of the GAL4 coding sequences, the epitope-tagged bait and library proteins were transcribed and translated without the GAL4 domains.

Proteins used for the co-immunopreciptation experiments were either synthesized in the coupled transcription-translation kit (TnT, Promega) or generated by first synthesizing the corresponding RNA and then adding this RNA to the translation mixture of the reticuloyte lysate (Promega). The translations were performed in the presence of [³⁵S]-methione to label the proteins. All reactions were carried out according to the manufacturer's instructions.

For the immunoprecipitation, the translation mixtures were incubated for 30 minutes at 30° C. with 1 μg of the antibody. After addition of buffer (PBS-KMT, 0.5% tween-20, 0.1% BSA) 5 μl of magnet proteinA beads (pretreated three times with buffer in order to equilibrate the beads) were added and incubated for one hour at 4° C. Magnetic proteinA beads were collected at the bottom of the tube using a magnetic device. Beads were washed three times with buffer, resuspended in buffer, transferred into a new reaction tube, and washed again. Finally, the supernatant was almost completely removed, and the beads were boiled in SDS sample buffer before performing SDS-PAGE. Samples were separated using a 4-20% Tris-glycine gel (Novex), and a phoshorimaging screen was used to detect the protein.

Analysis of Presence of ORF7 in Different Genetic Backgrounds of Pseudomonas aeruginosa

Genomic DNA from the different Pseudomonas strains PAO1, PA14, and PA37 was prepared using the DNeasy Tissue Kit (QIAGEN) according the manufacture's instructions. The presence of ORF7 was confirmed by PCR amplification of genomic DNA using Pwo DNA-polymerase (Roche) and the following oligonucleotides:

#63 forward
(5′-GCGGATCCCCATGATTAACAGTCATTTG-3)
and
#64 reverse
(5′-CCGTGATCACTATAGAAGGAAGGACGAC-3).

The PCR protocol is listed in Table 4.

TABLE 4

| | |
|---|---|
| 10xPwo-Puffer with MgSO₄ | 5.00 μl |
| 25 mM MgSO₄ | 4.00 μl |
| 10 mM dNTPs | 1.00 μl |
| oligo #63 forward | 0.25 μl |
| 100 pmol/μl | |
| oligo #64 | 0.25 μl |
| reverse100 pmol/μl | |
| genomic DNA | 1 μg |
| Pwo DNA-polymerase | 0.50 μl |
| 5 U/μl | |
| DMSO | 5.00 μl |
| water | |
| total | 50.0 μl |

The following PCR program in Perkin Elmer ThermoCycler 9600 was used: 30 seconds at 94° C.; 30 cycles in which each cycle included 30 seconds at 94° C., 30 seconds at 50° C., two minutes thirty seconds at 72° C.; and 7 minutes at 72° C. An aliquot of the PCR reaction was separated by agarose gel electrophoresis and analyzed. The DNA was stained with ethidium bromide.

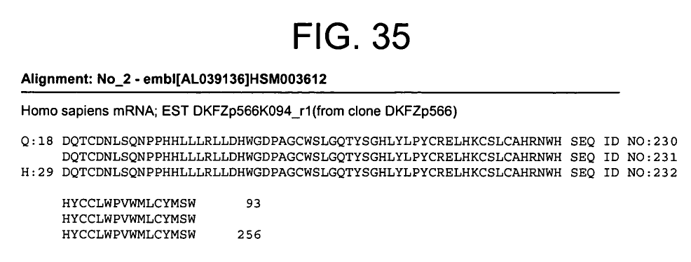

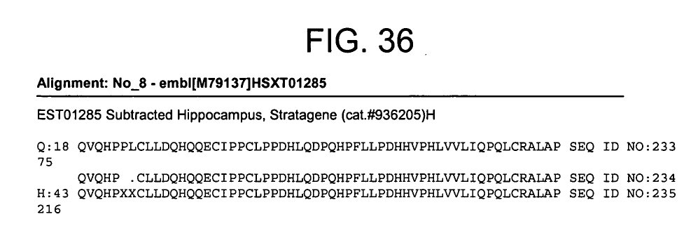

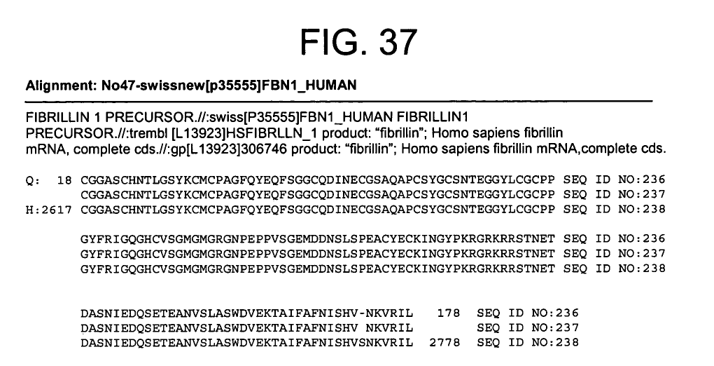

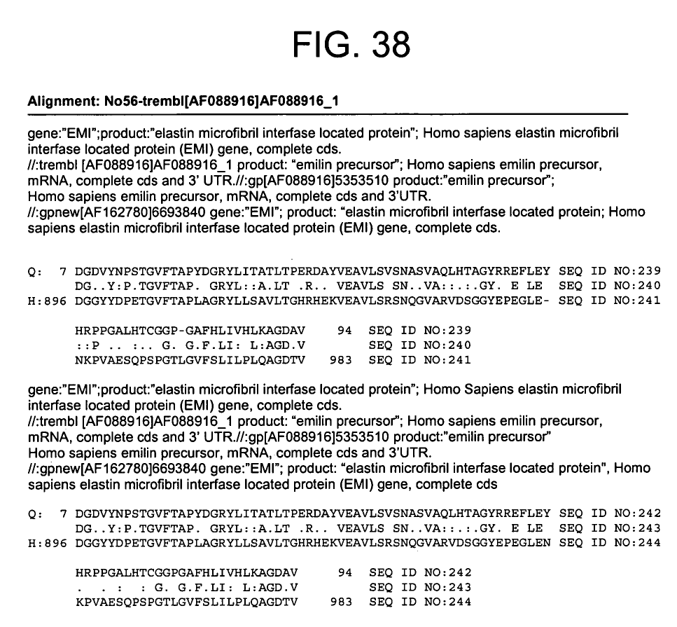

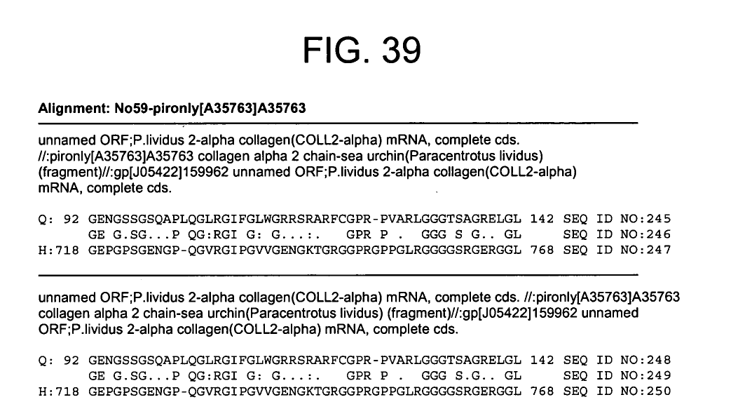

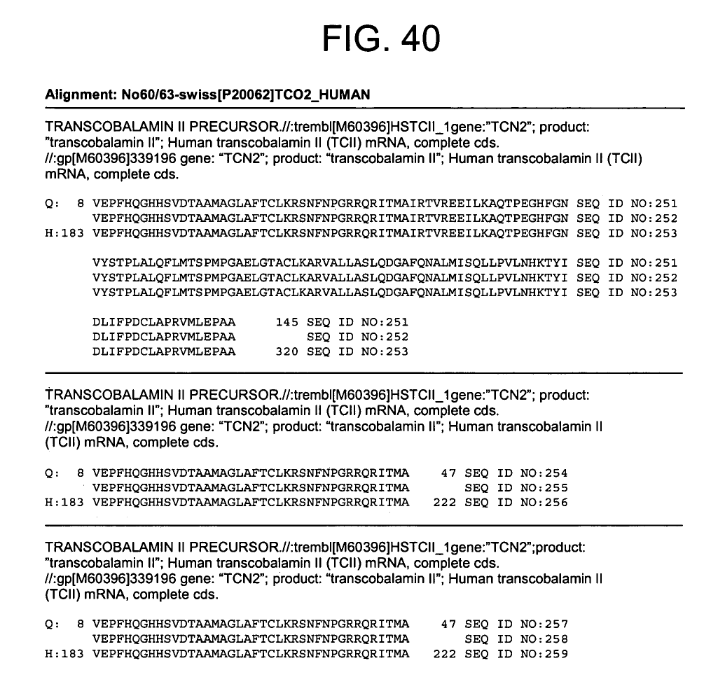

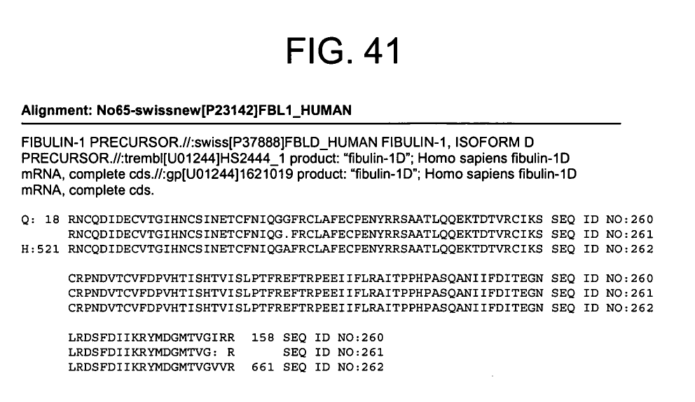

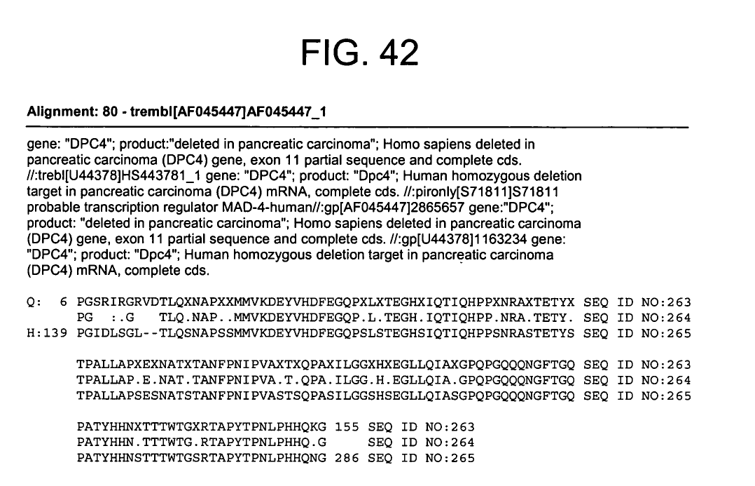

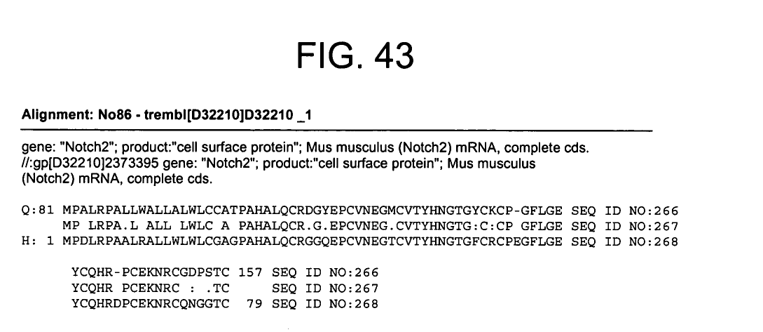

TABLE 5
Clones identified in the two-hybrid screen of ORF7 with a
human lung library (two independent screens, FIGS. 35-52)

| clone | clone | identified as | Homologous sequences |
|---|---|---|---|
| 2 | 1C3 | homologous to | SEQ ID NOs: 230-232 |
| | | Homo sapiens mRNA; EST | |
| | | DKFZp566K094_r1 | |
| | | (from clone DKFZp566) | |
| 8 | 1F8 | some homology to | SEQ ID NOs: 233-235 |
| | | EST01285 Subtracted | |
| | | Hippocampus, | |
| | | Stratagene (cat. #936205) | |
| 47 | 4C6 | direct assignment of | SEQ ID NOs: 236-238 |
| | | functionality | |
| | | by homology to | |
| | | FIBRILLIN 1 PRECURSOR | |
| 56 | 4E7 | clear assignment of | SEQ ID NOs: 239-241 |
| | | functionality by | SEQ ID NOs: 242-244 |
| | | homology to | |
| | | tremb1\|AF088916\|AF088916_1 | |
| | | emilin precursor; 50% identity in | |
| | | C1q-like domain | |
| 59 | 4F8 | potential assignment of | SEQ ID NOs: 245-247 |
| | | functionality by homology to | SEQ ID NOs: 248-250 |
| | | pironly\|A35763\|A35763 | |
| | | collagen alpha 2 | |
| | | chain - sea urchin | |
| | | (Paracentrotus lividus) | |
| | | (fragment) | |
| 60/63 | 4F10/ | Direct assignment of | SEQ ID NOs: 251-253 |
| | 4H8 | functionality by | SEQ ID NOs: 254-256 |
| | | homology to swiss\|P20062\| | |
| | | TCO2_HUMAN | SEQ ID NOs: 257-259 |
| | | TRANSCOBALAMIN II | |
| | | PRECURSOR | |
| 65 | 5A1 | Direct assignment of | SEQ ID NOs: 260-262 |
| | | functionality | |
| | | by homology to | |
| | | swissnew\|P23142\| | |
| | | FBL1_HUMAN | |
| | | FIBULIN-1 PRECURSOR. | |
| 80 | 5E7 | Clear assignment of | SEQ ID NOs: 263-265 |
| | | functionality | |
| | | by homology to | |
| | | tremb1\|AF045447\| | |
| | | AF045447_1 | |
| | | deleted in pancreatic | |
| | | carcinoma | |
| | | (DPC4) | |
| 86 | 5G1 | Clear assignment of | SEQ ID NOs: 266-268 |
| | | functionality | |
| | | by homology to | |
| | | tremb1\|D32210\|D32210_1 | |
| | | cell surface protein (Notch2) | |

Isolation of Additional Virulence Genes

Based on the nucleotide and amino acid sequences described herein, the isolation of additional coding sequences of virulence factors is made possible using standard strategies and techniques that are well known in the art. Any pathogenic cell can serve as the nucleic acid source for the molecular cloning of such a virulence gene, and these sequences are identified as ones encoding a protein exhibiting pathogenicity-associated structures, properties, or activities.

In one particular example of such an isolation technique, any one of the nucleotide sequences described herein may be used, together with conventional screening methods of nucleic acid hybridization screening. Such hybridization techniques and screening procedures are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 1997); Berger and Kimmel (supra); and Sambrook et al., Molecular Cloning: A Laboratory Manual, Cold Spring Harbor Laboratory Press, New York. In one particular example, all or part of any one of the polynucleotide sequences described herein may be used as a probe to screen a recombinant bacterial DNA library for genes having sequence identity to any one of the nucleic acids of the invention (SEQ ID NOs: 1-108, SEQ ID NOs: 119-120, and SEQ ID NOs: 281-282). Hybridizing sequences are detected by plaque or colony hybridization according to standard methods.

Alternatively, using all or a portion of the amino acid sequence of any one of the polypeptides described herein, one may readily design specific oligonucleotide probes, including degenerate oligonucleotide probes (i.e., a mixture of all possible coding sequences for a given amino acid sequence) for the amplification of additional nucleic acids encoding proteins of the invention. These oligonucleotides may be based upon the sequence of either DNA strand and any appropriate portion of a polynucleotide sequence of the invention (SEQ ID NOs: 1-108, SEQ ID NOs: 119-120, and SEQ ID NOs: 281-282). General methods for designing and preparing such probes are provided, for example, in Ausubel et al. (supra), and Berger and Kimmel, Guide to Molecular Cloning Techniques, 1987, Academic Press, New York. These oligonucleotides are useful for gene isolation, either through their use as probes capable of hybridizing to complementary sequences or as primers for various amplification techniques, for example, polymerase chain reaction (PCR) cloning strategies. If desired, a combination of different, detectably labeled oligonucleotide probes may be used for the screening of a recombinant DNA library. Such libraries are prepared according to methods well known in the art, for example, as described in Ausubel et al. (supra), or they may be obtained from commercial sources.

As discussed above, sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols. A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci. USA (1998) 85:8998).

Partial virulence sequences, e.g., sequence tags, are also useful as hybridization probes for identifying full-length sequences, as well as for screening databases for identifying previously unidentified related virulence genes.

Confirmation of a sequence's relatedness to a pathogenicity polypeptide may be accomplished by a variety of conventional methods including, but not limited to, functional complementation assays and sequence comparison of the gene and its expressed product. In addition, the activity of the gene product may be evaluated according to any of the techniques described herein, for example, the functional or immunological properties of its encoded product.

Once an appropriate sequence is identified, it is cloned according to standard methods and may be used, for example, for screening compounds that reduce the virulence of a pathogen.

Polypeptide Expression

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains, which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

Antibodies

To generate antibodies, a coding sequence for a polypeptide of the invention may be expressed as a C-terminal fusion with glutathione S-transferase (GST) (Smith et al., Gene 67:31-40, 1988). The fusion protein is purified on glutathione-Sepharose beads, eluted with glutathione, cleaved with thrombin (at the engineered cleavage site), and purified to the degree necessary for immunization of rabbits. Primary immunizations are carried out with Freund's complete adjuvant and subsequent immunizations with Freund's incomplete adjuvant. Antibody titres are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specific recognition by Western blot or immunoprecipitation analysis (by the methods described in Ausubel et al., supra). Antibodies which specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al., Nature Biotech 14:309-314, 1996).

Preferably, antibodies of the invention are produced using fragments of the polypeptide of the invention, which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Antibodies against any of the polypeptides described herein may be employed to treat bacterial infections.

Screening Assays

As discussed above, we have identified a number of P. aeruginosa virulence factors that are involved in pathogenicity and that may therefore be used to screen for compounds that reduce the virulence of that organism, as well as other microbial pathogens. For example, the invention provides methods of screening compounds to identify those which enhance (agonist) or block (antagonist) the action of a polypeptide or the gene expression of a nucleic acid sequence of the invention. The method of screening may involve high-throughput techniques.

Any number of methods are available for carrying out such screening assays. According to one approach, candidate compounds are added at varying concentrations to the culture medium of pathogenic cells expressing one of the nucleic acid sequences of the invention. Gene expression is then measured, for example, by standard Northern blot analysis (Ausubel et al., supra), using any appropriate fragment prepared from the nucleic acid molecule as a hybridization probe. The level of gene expression in the presence of the candidate compound is compared to the level measured in a control culture medium lacking the candidate molecule. If desired, the effect of candidate compounds may, in the alternative, be measured at the level of polypeptide production using the same general approach and standard immunological techniques, such as Western blotting or immunoprecipitation with an antibody specific for a pathogenicity factor. For example, immunoassays may be used to detect or monitor the expression of at least one of the polypeptides of the invention in a pathogenic organism. Polyclonal or monoclonal antibodies (produced as described above) which are capable of binding to such a polypeptide may be used in any standard immunoassay format (e.g., ELISA, Western blot, or RIA assay) to measure the level of the pathogenicity polypeptide.

As a specific example, pathogenic cells (e.g., Pseudomonas aeruginosa) that express a nucleic acid encoding a polypeptide substantially identical to the amino acid sequence of ORF7 (SEQ ID NO: 280) are cultured in the presence of a candidate compound (e.g., a peptide, polypeptide, synthetic organic molecule, naturally occurring organic molecule, nucleic acid molecule, or component thereof). In this regard, cells may endogenously express the polypeptide encoded by ORF7. Alternatively, cells may be genetically engineered by any standard technique known in the art (e.g., transfection and viral infection) to overexpress the polypeptide encoded by ORF7. The expression of the virulence factor encoded by the ORF7 nucleic acid is measured in these cells by means of Western blot analysis and subsequently compared to the level of expression of the same protein in control cells that have not been contacted by the candidate compound. A compound which promotes a decrease in the expression of the pathogenicity factor is considered useful in the invention. Given its ability to decrease the expression of a virulence factor, such a molecule may be used, for example, as a therapeutic to combat the pathogenicity of an infectious organism. Thus, if the pathogenic cell is Pseudomonas aeruginosa, the candidate compound identified by the present screening methods may be useful to treat humans and plants that are infected or are at risk of being infected with the strain of Pseudomonas aeruginosa which expresses the virulence factor to which the candidate compound is specific against. Accordingly, therapeutic compounds useful for treating disorders such as cystic fibrosis may be identified using the screening methods of the invention.

Alternatively, or in addition, candidate compounds may be screened for those which specifically bind to and inhibit a pathogenicity polypeptide of the invention. The efficacy of such a candidate compound is dependent upon its ability to interact with the pathogenicity polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate pathogenicity may be assayed by any standard assays (e.g., those described herein).

In one particular example, a candidate compound that binds to a pathogenicity polypeptide may be identified using a chromatography-based technique. For example, a recombinant polypeptide of the invention, such as the polypeptide encoded by ORF7, may be purified by standard techniques from cells engineered to express the polypeptide (e.g., those described above) and may be immobilized on a column. A solution of candidate compounds is then passed through the column, and a compound specific for the pathogenicity polypeptide is identified on the basis of its ability to bind to the pathogenicity polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to render a pathogen less virulent (e.g., as described herein). Compounds isolated by this approach may also be used, for example, as therapeutics to treat or prevent the onset of a pathogenic infection, disease, or both. Compounds which are identified as binding to pathogenicity polypeptides with an affinity constant less than or equal to 10 mM are considered particularly useful in the invention.

Alternatively, a candidate compound may be contacted with two proteins, the first protein being a substantially pure polypeptide such as an isolated bacterial virulence factor (e.g., any one of SEQ ID NOs: 127-229 and 278-280) and the second protein (e.g., a human lung protein having an amino acid sequence of any one of SEQ ID NOs: 269-277) being a polypeptide that binds the first protein under conditions that allow binding. In this respect, the second protein may be any protein that under normal conditions binds the first protein, or alternatively may be an antibody or an antibody fragment. For example, the candidate compound may be contacted in vitro with the polypeptide encoded by ORF7 which is substantially identical to the amino acid sequence of SEQ ID NO: 280 or SEQ ID NO: 278 and a human protein which is substantially identical to any one of the amino acid sequence of SEQ ID NOs: 269-277. Under the appropriate conditions, the polypeptide encoded by ORF7 binds a human protein, such as a lung protein. According to this particular screening method, the interaction between these two proteins is measured following the addition of a candidate compound. Thus, a decrease in the binding of the first polypeptide to the second polypeptide following the addition of the candidate compound (relative to such binding in the absence of the compound) would identify the candidate compound as having the ability to bind the first protein and as having the ability to inhibit the virulence of a pathogenic organism. Contacting of the candidate compound with the two proteins may occur in a cell-free system or using a yeast two-hybrid system. If desired, the first protein or the candidate compound may be immobilized on a support as described above or may have a detectable group. Alternatively, the candidate compound may be expressed on the surface of a phage or may be expressed using RNA display according to standard methods.

Potential antagonists include organic molecules, peptides, peptide mimetics, polypeptides, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented. Other potential antagonists include antisense molecules.

Each of the DNA sequences provided herein may also be used in the discovery and development of antipathogenic compounds (e.g., antibiotics). The encoded protein, upon expression, can be used as a target for the screening of antibacterial drugs. Additionally, the DNA sequences encoding the amino terminal regions of the encoded protein or Shine-Dalgarno or other translation facilitating sequences of the respective mRNA can be used to construct antisense sequences to control the expression of the coding sequence of interest.

The invention also provides the use of the polypeptide, polynucleotide, or inhibitor to Interfere with the initial physical interaction between a pathogen and mammalian host responsible for infection, for example. In particular the molecules of the invention, for example, may be used, for example: in the prevention of adhesion and colonization of bacteria and binding to mammalian extracellular matrix proteins; to extracellular matrix proteins in wounds; to block mammalian cell invasion; or to block the normal progression of pathogenesis.

The antagonists and agonists of the invention may be employed, for instance, to inhibit and treat a variety of bacterial infections.

Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics (e.g, antibiotics).

Test Compounds and Extracts

In general, compounds capable of reducing pathogenic virulence are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N. H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Pharmaceutical Therapeutics and Plant Protectants

The invention provides a simple means for identifying compounds (including peptides, small molecule inhibitors, and mimetics) capable of inhibiting the pathogenicity or virulence of a pathogen. Accordingly, a chemical entity discovered to have medicinal or agricultural value using the methods described herein are useful as either drugs, plant protectants, or as information for structural modification of existing anti-pathogenic compounds, e.g., by rational drug design. Such methods are useful for screening compounds having an effect on a variety of pathogens including, but not limited to, bacteria, viruses, fungi, annelids, nematodes, platyhelminthes, and protozoans. Examples of pathogenic bacteria include, without limitation, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the animal with antagonists, which disrupt, suppress, attenuate, or neutralize the biological events associated with a pathogenicity polypeptide. Preferable routes of administration include, for example, subcutaneous, intravenous, interperitoneally, intramuscular, or intradermal injections, which provide continuous, sustained levels of the drug in the patient. Treatment of human patients or other animals will be carried out using a therapeutically effective amount of an anti-pathogenic agent in a physiologically-acceptable carrier. Suitable carriers and their formulation are described, for example, in Remington's Pharmaceutical Sciences by E. W. Martin. The amount of the anti-pathogenic agent (e.g., an antibiotic) to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation. For example, for systemic administration a compound is administered typically in the range of 0.1 ng-10 g/kg body weight.

For agricultural uses, the compositions or agents identified using the methods disclosed herein may be used as chemicals applied as sprays or dusts on the foliage of plants. Typically, such agents are to be administered on the surface of the plant in advance of the pathogen in order to prevent infection. Seeds, bulbs, roots, tubers, and corms are also treated to prevent pathogenic attack after planting by controlling pathogens carried on them or existing in the soil at the planting site. Soil to be planted with vegetables, ornamentals, shrubs, or trees can also be treated with chemical fumigants for control of a variety of microbial pathogens. Treatment is preferably done several days or weeks before planting. The chemicals can be applied by either a mechanized route, e.g., a tractor or with hand applications. In addition, chemicals identified using the methods of the assay can be used as disinfectants.

### Other Embodiments

In general, the invention includes any nucleic acid sequence which may be isolated as described herein or which is readily isolated by homology screening or PCR amplification using the nucleic acid sequences of the invention. Also included in the invention are polypeptides which are modified in ways which do not abolish their pathogenic activity (assayed, for example as described herein). Such changes may include certain mutations, deletions, insertions, or post-translational modifications, or may involve the inclusion of any of the polypeptides of the invention as one component of a larger fusion protein. Also, included in the invention are polypeptides that have lost their pathogenicity.

Thus, in other embodiments, the invention includes any protein which is substantially identical to a polypeptide of the invention. Such homologs include other substantially pure naturally-occurring polypeptides as well as allelic variants; natural mutants; induced mutants; proteins encoded by DNA that hybridizes to any one of the nucleic acid sequences of the invention under high stringency conditions or, less preferably, under low stringency conditions (e.g., washing at 2×SSC at 40° C. with a probe length of at least 40 nucleotides); and proteins specifically bound by antisera of the invention.

The invention further includes analogs of any naturally-occurring polypeptide of the invention. Analogs can differ from the naturally-occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs, which contain residues other than L-amino acids, e.g., D-amino acids or non-naturally occurring or synthetic amino acids, e.g., or amino acids.

In addition to full-length polypeptides, the invention also includes fragments of any one of the polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Furthermore, the invention includes nucleotide sequences that facilitate specific detection of any of the nucleic acid sequences of the invention. Thus, for example, nucleic acid sequences described herein or fragments thereof may be used as probes to hybridize to nucleotide sequences by standard hybridization techniques under conventional conditions. Sequences that hybridize to a nucleic acid sequence coding sequence or its complement are considered useful in the invention. Sequences that hybridize to a coding sequence of a nucleic acid sequence of the invention or its complement and that encode a polypeptide of the invention are also considered useful in the invention. As used herein, the term “fragment,” as applied to nucleic acid sequences, means at least 5, 10, 20, 30, 50, 100, 200, 300, 400 contiguous nucleotides, preferably at least 500 contiguous nucleotides, more preferably at least 600, 700, 800, 900 to 1000 contiguous nucleotides, and most preferably at least 1100, 1200, 1300, 1400, 1500, 1600, 1800, 2000, or more contiguous nucleotides. Fragments of nucleic acid sequences can be generated by methods known to those skilled in the art.

The invention further provides a method for inducing an immunological response in an individual, particularly a human, which includes inoculating the individual with, for example, any of the polypeptides (or a fragment or analog thereof or fusion protein) of the invention to produce an antibody and/or a T cell immune response to protect the individual from infection, especially bacterial infection (e.g., a Pseudomonas aeruginosa infection). The invention further includes a method of inducing an immunological response in an individual which includes delivering to the individual a nucleic acid vector to direct the expression of a polypeptide described herein (or a fragment or fusion thereof) in order to induce an immunological response.

The invention also includes vaccine compositions including the polypeptides or nucleic acid sequences of the invention. For example, the polypeptides of the invention may be used as an antigen for vaccination of a host to produce specific antibodies which protect against invasion of bacteria, for example, by blocking the production of phenazines. The invention therefore includes a vaccine formulation which includes an immunogenic recombinant polypeptide of the invention together with a suitable carrier.

The invention further provides compositions (e.g., nucleotide sequence probes), polypeptides, antibodies, and methods for the diagnosis of a pathogenic condition.

All publications and patent applications mentioned in this specification are herein incorporated by reference to the same extent as if each independent publication or patent application was specifically and individually indicated to be incorporated by reference.

Other embodiments are within the scope of the claims.

1. An isolated nucleic acid encoding a protein comprising an amino acid sequence at least 25% identical to any one of the amino acid sequences of SEQ ID NOs: 127-229 and SEQ ID NOs: 278-280 over the entire length of said amino acid sequence of SEQ ID NOs: 127-229 and SEQ ID NOs: 278-280 wherein said protein is a pathogenic virulence factor.

2. The nucleic acid of claim 1, encoding a protein comprising an amino acid sequence at least 50% identical to any one of the amino acid sequences of SEQ ID NOs: 127-229 and SEQ ID NOs: 278-280 over the entire length of said amino acid sequence of SEQ ID NOs: 127-229 and SEQ ID NOs: 278-280.

3. The nucleic acid of claim 1, comprising a polynucleotide sequence at least 80% identical to any one of the polynucleotide sequences of SEQ ID NOs: 1-108, SEQ ID NOs: 119-120, and SEQ ID NOs: 281-282 over the entire length of said polynucleotide sequence of SEQ ID NOs: 1-108, SEQ ID NOs: 119-120, and SEQ ID NOs: 281-282.

4. The nucleic acid of claim 3, comprising a polynucleotide sequence identical to any one of the polynucleotide sequences of SEQ ID NOs: 1-108, SEQ ID NOs: 119-120, and SEQ ID NOs: 281-282.

5. The nucleic acid of claim 1, wherein said protein binds a human protein.

6. The nucleic acid of claim 5, wherein said human protein is a lung protein.

7. The nucleic acid of claim 1, wherein said protein has an Arg-Gly-Asp motif.

8. An isolated nucleic acid comprising a polynucleotide sequence at least 65% identical to the corresponding region of any one of the polynucleotide sequences of SEQ ID NOs: 1-108, SEQ ID NOs: 119-120, and SEQ ID NOs: 281-282 or the complement thereof.

9. An isolated nucleic acid that hybridizes at high stringency to a region of any one of the polynucleotide sequences of SEQ ID NOs: 1-108, SEQ ID NOs: 119-120, and SEQ ID NOs: 281-282 or the complement thereof.

10. The nucleic acid of claim 9, having a sequence complementary to at least 50% of at least 60 nucleotides of any one of the polynucleotide sequences of SEQ ID NOs: 1-108, SEQ ID NOs: 119-120, and SEQ ID NOs: 281-282.

11. The nucleic acid of claim 8, comprising at least 100 contiguous nucleotides of any one of the polynucleotide sequences of SEQ ID NOs: 1-108, SEQ ID NOs: 119-120, and SEQ ID NOs: 281-282.

12. The nucleic acid of claim 8, encoding a protein or protein fragment that binds a human lung protein.

13. The nucleic acid of claim 8, encoding a protein or protein fragment comprising an Arg-Gly-Asp motif.

14. A vector comprising a nucleic acid of claim 1.

15. A substantially pure protein comprising an amino acid sequence at least 25% identical to any one of the amino acid sequences of SEQ ID NOs: 127-229 and SEQ ID NOs: 278-280, wherein said protein is a pathogenic virulence factor.

16. The protein of claim 15, comprising an amino acid sequence at least 50% identical to any one of the amino acid sequences of SEQ ID NOs: 127-229 and SEQ ID NOs: 278-280.

17. The protein of claim 16, comprising an amino acid sequence identical to any one of the amino acid sequences of SEQ ID NOs: 127-229 and SEQ ID NOs: 278-280.

18. The protein of claim 15, comprising at least 100 contiguous amino acids of any one of the amino acid sequences of SEQ ID NOs: 127-229 and SEQ ID NOs: 278-280.

19. The protein of claim 18, wherein said protein is immunogenic.

20. The protein of claim 15, wherein said protein binds a human protein.

21. The protein of claim 20 wherein said human protein is a lung protein.

22. The protein of claim 15, comprising an Arg-Gly-Asp motif.

23. An isolated nucleic acid encoding a protein comprising an amino acid sequence at least 25% identical to any one of the amino acid sequences of SEQ ID NOs: 269-277 wherein said protein binds the polypeptide (SEQ ID NO: 278 or SEQ ID NO: 280) encoded by the nucleic acid sequence of ORF7 (SEQ ID NO: 119 or SEQ ID NO: 281).

24. The nucleic acid of claim 23, wherein said protein is expressed in the lungs of a mammal.

25. A substantially pure protein comprising an amino acid sequence at least 25% identical to any one of the amino acid sequences of SEQ ID NOs: 269-277, wherein said protein binds the polypeptide (SEQ ID NO: 278 or SEQ ID NO: 280) encoded by the nucleic acid sequence of ORF7 (SEQ ID NO: 119 or SEQ ID NO: 281).

26. The protein of claim 25, wherein said protein is expressed in the lungs of a mammal.
